(19) United States
(12) Patent Application Publication
(10) Pub. No.: US 2019/0165282 A1
(43) Pub. Date: May 30, 2019

(54) HETEROCYCLIC COMPOUNDS COMPRISING DIBENZOFURAN AND/OR DIBENZOTHIOPHENE STRUCTURES

(71) Applicant: Merck Patent GmbH, Darmstadt (DE)

(72) Inventors: Amir Parham, Frankfurt am Main (DE); Thomas Eberle, Landau (DE); Anja Jatsch, Frankfurt am Main (DE); Tobias Grossmann, Darmstadt (DE); Jonas Kroeber, Frankfurt am Main (DE)

(73) Assignee: Merck Patent GmbH, Darmstadt (DE)

(21) Appl. No.: 16/092,860

(22) PCT Filed: Apr. 6, 2017
(86) PCT No.: PCT/EP2017/058176

§ 371 Date: Oct. 11, 2018

(30) Foreign Application Priority Data
Apr. 11, 2016 (EP) 16164690.6

(51) Int. Cl. H01L 51/00; C07D 409/10; C07D 405/10; C07D 405/14; C07D 409/14

(52) U.S. Cl. CPC H01L 51/0072; C07D 409/10; H01L 51/0074; H01L 51/0073; H01L 51/5016; H01L 51/0067; C07D 405/14; C07D 409/14; C07D 405/10

(57) ABSTRACT

The present invention describes dibenzofuran and dibenzothiophene derivatives substituted by electron-transporting groups, especially for use as triplet matrix materials in electronic devices. The invention further relates to a process for preparing the compounds of the invention and to electronic devices comprising these.

The present invention describes dibenzofuran and dibenzothiophene derivatives substituted by electron-transporting groups, especially for use in electronic devices. The invention further relates to a process for preparing the compounds of the invention and to electronic devices comprising these compounds.

The structure of organic electroluminescent devices (OLEDs) in which organic semiconductors are used as functional materials is described, for example, in U.S. Pat. Nos. 4,539,507, 5,151,629, EP 0676461 and WO 98/27136. Emitting materials used are frequently organometallic complexes which exhibit phosphorescence. For quantum-mechanical reasons, up to four times the energy efficiency and power efficiency is possible using organometallic compounds as phosphorescent emitters. In general terms, there is still a need for improvement in OLEDs, especially also in OLEDs which exhibit phosphorescence, for example with regard to efficiency, operating voltage and the lifetime.

The properties of phosphorescent OLEDs are not just determined by the triplet emitters used. More particularly, the other materials used, for example matrix materials, are also of particular significance here. Improvements to these materials can thus also lead to distinct improvements in the OLED properties.

According to the prior art, among other materials, carbazole derivatives (for example according to WO 2014/015931), indolocarbazole derivatives (for example according to WO 2007/063754 or WO 2008/056746) or indenocarbazole derivatives (for example according to WO 2010/136109 or WO 2011/000455), especially those substituted by electron-deficient heteroaromatics such as triazine, are used as matrix materials for phosphorescent emitters. In addition, for example, bisdibenzofuran derivatives (for example according to EP 2301926) are used as matrix materials for phosphorescent emitters. WO 2013/077352 discloses triazine derivatives in which the triazine group is bonded to a dibenzofuran group via a divalent arylene group. These compounds are described as hole blocker materials. No use of these materials as host for phosphorescent emitters is disclosed. Moreover, EP 2752902 discloses heterocyclic compounds having dibenzofuran and dibenzothiophene structures. However, the dibenzofuran and dibenzothiophene structures have only one bonding site to other heterocycles, i.e. are only monosubstituted. Similar compounds are additionally known from KR 20130115160.

In general terms, in the case of these materials for use as matrix materials, there is still need for improvement, particularly in relation to the lifetime, but also in relation to the efficiency and operating voltage of the device.

It is an object of the present invention to provide compounds suitable for use in a phosphorescent or fluorescent OLED, especially as matrix material. More particularly, it is an object of the present invention to provide matrix materials which are suitable for red-, yellow- and green-phosphorescing OLEDs and possibly also for blue-phosphorescing OLEDs, and which lead to long lifetime, good efficiency and low operating voltage. Particularly the properties of the matrix materials too have an essential influence on the lifetime and efficiency of the organic electroluminescent device.

Moreover, the compounds should be processible in a very simple manner, and especially exhibit good solubility and film formation. For example, the compounds should exhibit elevated oxidation stability and an improved glass transition temperature.

A further object can be considered that of providing electronic devices having excellent performance very inexpensively and in constant quality.

Furthermore, it should be possible to use or adapt the electronic devices for many purposes. More particularly, the performance of the electronic devices should be maintained over a broad temperature range.

It has been found that, surprisingly, devices containing compounds comprising structures of the formula (I) below have improvements over the prior art, especially when used as matrix material for phosphorescent dopants.

The present invention therefore provides a compound comprising structures of the following formula (I):

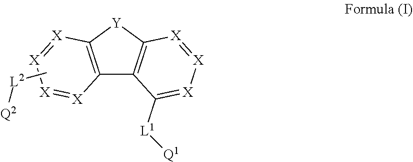

where the symbols used are as follows:
Y is O or S;
X is the same or different at each instance and is N or CR¹, preferably CR¹, with the proviso that not more than two of the X groups in one cycle are N, and C is the attachment site of the L² group;
Q¹, Q² is in each case independently an electron-transporting group;
L¹, L² is a bond or an aromatic or heteroaromatic ring system which has 5 to 30 aromatic ring atoms and may be substituted by one or more R¹ radicals;
R¹ is the same or different at each instance and is H, D, F, Cl, Br, I, B(OR²)₂, CHO, C(═O)R², CR²═C(R²)₂, CN, C(═O)OR², C(═O)N(R²)₂, Si(R²)₃, N(R²)₂, NO₂, P(═O)(R²)₂, OSO₂R², OR², S(═O)R², S(═O)₂R², a straight-chain alkyl, alkoxy or thioalkoxy group having 1 to 40 carbon atoms or a branched or cyclic alkyl, alkoxy or thioalkoxy group having 3 to 40 carbon atoms, each of which may be substituted by one or more R² radicals, where one or more nonadjacent CH₂ groups may be replaced by —R²C═CR²—, —C≡C—, Si(R²)₂, C═O, C═S, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 40 aromatic ring atoms and may be substituted in each case by one or more R² radicals, or an aryloxy or heteroaryloxy group which has 5 to 40 aromatic ring atoms and may be substituted by one or more R² radicals, or a combination of these systems; at the same time, two or more adjacent R¹ substituents together may also form a mono- or polycyclic, aliphatic or aromatic ring system;
R² is the same or different at each instance and is H, D, F, Cl, Br, I, B(OR³)₂, CHO, C(═O)R³, CR³═C(R³)₂, ON, C(═O)OR³, C(═O)N(R³)₂, Si(R³)₃, N(R³)₂, NO₂, P(═O)(R³)₂, OSO₂R³, OR³, S(═O)R³, S(═O)₂R³, a straight-chain alkyl, alkoxy or thioalkoxy group having 1 to 40 carbon atoms or a branched or cyclic alkyl, alkoxy or thioalkoxy group having 3 to 40 carbon atoms, each of which may be substituted by one or more R³ radicals, where one or more nonadjacent CH₂ groups may be replaced by —R³C═CR³—, —C≡C—, Si(R³)₂, C═O, C═S, C═NR³, —C(═O)O—, —C(═O)NR³—, NR³, P(═O)(R³), —O—, —S—, SO or SO₂ and where one or more hydrogen atoms may be replaced by D, F, Cl, Br, I, CN or NO₂, or an aromatic or heteroaromatic ring system which has 5 to 40 aromatic ring atoms and may be substituted in each case by one or more R³ radicals, or an aryloxy or heteroaryloxy group which has 5 to 40 aromatic ring atoms and may be substituted by one or more R³ radicals, or a combination of these systems; at the same time, two or more adjacent R² substituents together may also form a mono- or polycyclic, aliphatic or aromatic ring system;
R³ is the same or different at each instance and is H, D, F or an aliphatic, aromatic and/or heteroaromatic hydrocarbyl radical having 1 to 20 carbon atoms, in which hydrogen atoms may also be replaced by F; at the same time, two or more adjacent R³ substituents together may also form a mono- or polycyclic, aliphatic or aromatic ring system.

Adjacent carbon atoms in the context of the present invention are carbon atoms bonded directly to one another. In addition, “adjacent radicals” in the definition of the radicals means that these radicals are bonded to the same carbon atom or to adjacent carbon atoms. These definitions apply correspondingly, inter alia, to the terms “adjacent groups” and “adjacent substituents”.

The wording that two or more radicals together may form a ring, in the context of the present description, shall be understood to mean, inter alia, that the two radicals are joined to one another by a chemical bond with formal elimination of two hydrogen atoms. This is illustrated by the following scheme:

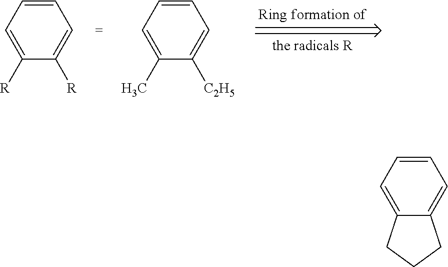

In addition, however, the abovementioned wording shall also be understood to mean that, if one of the two radicals is hydrogen, the second radical binds to the position to which the hydrogen atom was bonded, forming a ring. This shall be illustrated by the following scheme:

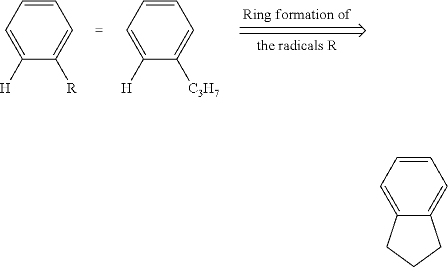

A fused aryl group in the context of the present invention is a group in which two or more aromatic groups are fused, i.e. annelated, to one another along a common edge, such that, for example, two carbon atoms belong to the at least two aromatic or heteroaromatic rings, as, for example, in naphthalene. By contrast, for example, fluorene is not a fused aryl group in the context of the present invention, since the two aromatic groups in fluorene do not have a common edge.

An aryl group in the context of this invention contains 6 to 40 carbon atoms; a heteroaryl group in the context of this invention contains 2 to 40 carbon atoms and at least one heteroatom, with the proviso that the sum total of carbon atoms and heteroatoms is at least 5. The heteroatoms are preferably selected from N, O and/or S. An aryl group or heteroaryl group is understood here to mean either a simple aromatic cycle, i.e. benzene, or a simple heteroaromatic cycle, for example pyridine, pyrimidine, thiophene, etc., or a fused aryl or heteroaryl group, for example naphthalene, anthracene, phenanthrene, quinoline, isoquinoline, etc.

An aromatic ring system in the context of this invention contains 6 to 40 carbon atoms in the ring system. A heteroaromatic ring system in the context of this invention contains 1 to 40 carbon atoms and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for two or more aryl or heteroaryl groups to be interrupted by a nonaromatic unit (preferably less than 10% of the atoms other than H), for example a carbon, nitrogen or oxygen atom or a carbonyl group. For example, systems such as 9,9′-spirobifluorene, 9,9-diarylfluorene, triarylamine, diaryl ethers, stilbene, etc. shall thus also be regarded as aromatic ring systems in the context of this invention, and likewise systems in which two or more aryl groups are interrupted, for example, by a linear or cyclic alkyl group or by a silyl group. In addition, systems in which two or more aryl or heteroaryl groups are bonded directly to one another, for example biphenyl, terphenyl, quaterphenyl or bipyridine, shall likewise be regarded as an aromatic or heteroaromatic ring system.

A cyclic alkyl, alkoxy or thioalkoxy group in the context of this invention is understood to mean a monocyclic, bicyclic or polycyclic group.

In the context of the present invention, a C₁- to C₂₀-alkyl group in which individual hydrogen atoms or CH₂ groups may also be substituted by the abovementioned groups is understood to mean, for example, the methyl, ethyl, n-propyl, i-propyl, cyclopropyl, n-butyl, i-butyl, s-butyl, t-butyl, cyclobutyl, 2-methylbutyl, n-pentyl, s-pentyl, t-pentyl, 2-pentyl, neopentyl, cyclopentyl, n-hexyl, s-hexyl, t-hexyl, 2-hexyl, 3-hexyl, neohexyl, cyclohexyl, 1-methylcyclopentyl, 2-methylpentyl, n-heptyl, 2-heptyl, 3-heptyl, 4-heptyl, cycloheptyl, 1-methylcyclohexyl, n-octyl, 2-ethylhexyl, cyclooctyl, 1-bicyclo[2.2.2]octyl, 2-bicyclo[2.2.2]octyl, 2-(2,6-dimethyl)octyl, 3-(3,7-dimethyl)octyl, adamantyl, trifluoromethyl, pentafluoroethyl, 2,2,2-trifluoroethyl, 1,1-dimethyl-n-hex-1-yl, 1,1-dimethyl-n-hept-1-yl, 1,1-dimethyl-n-oct-1-yl, 1,1-dimethyl-n-dec-1-yl, 1,1-dimethyl-n-dodec-1-yl, 1,1-dimethyl-n-tetradec-1-yl, 1,1-dimethyl-n-hexadec-1-yl, 1,1-dimethyl-n-octadec-1-yl, 1,1-diethyl-n-hex-1-yl, 1,1-diethyl-n-hept-1-yl, 1,1-diethyl-n-oct-1-yl, 1,1-diethyl-n-dec-1-yl, 1,1-diethyl-n-dodec-1-yl, 1,1-diethyl-n-tetradec-1-yl, 1,1-diethyl-n-hexadec-1-yl, 1,1-diethyl-n-octadec-1-yl, 1-(n-propyl)cyclohex-1-yl, 1-(n-butyl)cyclohex-1-yl, 1-(n-hexyl)cyclohex-1-yl, 1-(n-octyl)cyclohex-1-yl and 1-(n-decyl)cyclohex-1-yl radicals. An alkenyl group is understood to mean, for example, ethenyl, propenyl, butenyl, pentenyl, cyclopentenyl, hexenyl, cyclohexenyl, heptenyl, cycloheptenyl, octenyl, cyclooctenyl or cyclooctadienyl. An alkynyl group is understood to mean, for example, ethynyl, propynyl, butynyl, pentynyl, hexynyl, heptynyl or octynyl. A C₁- to C₄₀-alkoxy group is understood to mean, for example, methoxy, trifluoromethoxy, ethoxy, n-propoxy, i-propoxy, n-butoxy, i-butoxy, s-butoxy, t-butoxy or 2-methylbutoxy.

An aromatic or heteroaromatic ring system which has 5-40 aromatic ring atoms and may also be substituted in each case by the abovementioned radicals and which may be joined to the aromatic or heteroaromatic system via any desired positions is understood to mean, for example, groups derived from benzene, naphthalene, anthracene, benzanthracene, phenanthrene, benzophenanthrene, pyrene, chrysene, perylene, fluoranthene, benzofluoranthene, naphthacene, pentacene, benzopyrene, biphenyl, biphenylene, terphenyl, terphenylene, fluorene, spirobifluorene, dihydrophenanthrene, dihydropyrene, tetrahydropyrene, cis- or trans-indenofluorene, cis- or trans-monobenzoindenofluorene, cis- or trans-dibenzoindenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, indolocarbazole, indenocarbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

In a preferred configuration, the compounds of the invention may form a structure of formula (II)

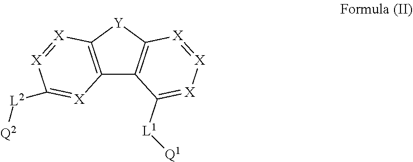

in which
the symbols X, Y, L¹, L², Q¹ and Q² have the definition given above, especially for formula (I).

Furthermore, preference is given to compounds which are characterized in that, in formulae (I) and (II), not more than two X groups are N and preferably not more than one X group is N, and preferably all X are CR¹, where preferably at most 4, more preferably at most 3 and especially preferably at most 2 of the CR¹ groups that X represents are not the CH group.

It may further be the case that the R¹ radicals of the X groups in the formula (I) and/or (II) do not form a fused ring system with the ring atoms of the benzofuran and/or benzothiophene structure. This includes the formation of a fused ring system with possible R², R³ substituents which may be bonded to the R¹ radicals. It may preferably be the case that the R¹ radicals of the X groups in the formula (I) and/or (II) do not form a ring system with the ring atoms of the benzofuran and/or benzothiophene structure. This includes the formation of a ring system with possible R², R³ substituents which may be bonded to the R¹ radicals.

Preferably, the compounds of the invention may comprise structures of formula (Ia)

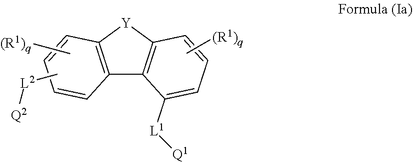

in which the symbols Y, R¹, L¹, L², Q¹ and Q² have the definition detailed above, especially for formula (I) and/or (II), and q is 0, 1 or 2, preferably 0 or 1.

In a further configuration, the compounds of the invention may comprise structures of formula (IIa)

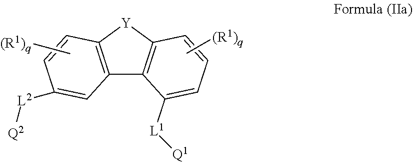

in which the symbols Y, R¹, L¹, L², Q¹ and Q² have the definition detailed above, especially for formula (I) and/or (II), and q is 0, 1 or 2, preferably 0 or 1.

It may additionally be the case that the R¹ substituents of the benzofuran and/or benzothiophene structure in the formulae (Ia) and/or (IIa) do not form a fused ring system with the ring atoms of the benzofuran and/or benzothiophene structure. This includes the formation of a fused ring system with possible R², R³ substituents which may be bonded to the R¹ radicals. It may preferably be the case that the R¹ substituents of the benzofuran and/or benzothiophene structure in the formulae (Ia) and/or (IIa) do not form a ring system with the ring atoms of the benzofuran and/or benzothiophene structure. This includes the formation of a ring system with possible R², R³ substituents which may be bonded to the R¹ radicals.

In a preferred configuration, compounds comprising structures of formula (I), (Ia), (II) and/or (IIa) can be represented by structures of the formula (I), (Ia), (II) and/or (IIa), and so particular preference is given to compounds of formula (I), (Ia), (II) and/or (IIa). Preferably, compounds comprising structures of formula (I), (Ia), (II) and/or (IIa) have a molecular weight of not more than 5000 g/mol, preferably not more than 4000 g/mol, particularly preferably not more than 3000 g/mol, especially preferably not more than 2000 g/mol and most preferably not more than 1200 g/mol.

In addition, it is a feature of preferred compounds of the invention that they are sublimable. These compounds generally have a molar mass of less than about 1200 g/mol.

The Q¹ and Q² groups are electron-transporting groups. These groups are widely known in the technical field and promote the ability of compounds to transport and/or conduct electrons.

Moreover, compounds of formula (I) show surprising advantages where, in formulae (I), (II), (Ia) and/or (IIa), the Q¹ and/or Q² group comprises at least one structure selected from the group of pyridines, pyrimidines, pyrazines, pyridazines, triazines, quinazolines, quinoxalines, quinolines, isoquinolines, imidazoles and/or benzimidazoles.

Preference is further given to compounds which are characterized in that at least one and preferably both of the Q¹ and/or Q² groups are a heteroaromatic ring system having 5 to 24 ring atoms, where the ring atoms comprise at least one nitrogen atom and the ring system may be substituted by one or more R¹ radicals, where R¹ has the definition detailed above, especially for formula (I).

In a further configuration, it may be the case that at least one and preferably both of the Q¹ and/or Q² groups detailed, inter alia, in the formulae (I), (II), (Ia) and/or (IIa) represents a heteroaromatic ring system, where the ring atoms comprise 1 to 4 nitrogen atoms and the ring system may be substituted by one or more R¹ radicals, where R¹ has the definition detailed above, especially for formula (I).

In addition, it may be the case that at least one and preferably both of the Q¹ and/or Q² groups detailed, inter alia, in the formulae (I), (II), (Ia) and/or (IIa) represents a heteroaromatic ring system which has 6 to 10 ring atoms and may be substituted by one or more R¹ radicals, where R¹ has the definition detailed above, especially for formula (I).

Preferably, the Q¹ and/or Q² groups detailed, inter alia, in the formulae (I), (II), (Ia) and/or (IIa) may be selected from structures of the formulae (Q-1), (Q-2) and/or (Q-3)

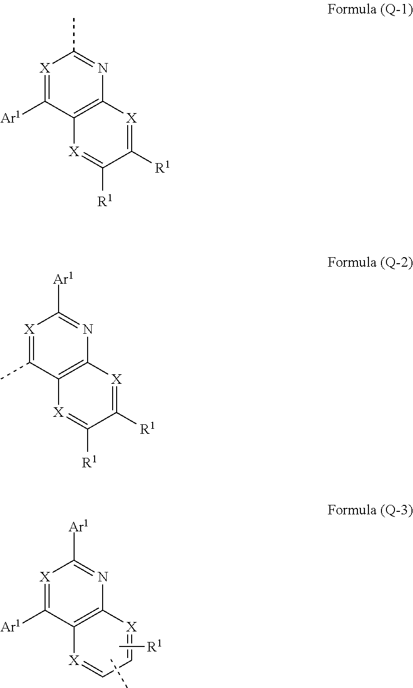

where the symbols X and R¹ have the definition given above, inter alia, for formula (I), the dotted bond marks the attachment position and Ar¹ is an aromatic or heteroaromatic ring system which has 6 to 40 carbon atoms and may be substituted in each case by one or more R² radicals, an aryloxy group which has 5 to 60 aromatic ring atoms and may be substituted by one or more R² radicals, or an aralkyl group which has 5 to 60 aromatic ring atoms and may be substituted in each case by one or more R² radicals, where two or more adjacent R¹ and/or R² substituents may optionally form a mono- or polycyclic aliphatic ring system which may be substituted by one or more R³ radicals, where R² and R³ have the definition given above, especially for formula (I).

In a further embodiment, the Q¹ and/or Q² groups detailed, inter alia, in the formulae (I), (II), (Ia) and/or (IIa) are selected from structures of the formulae (Q-4), (Q-5), (Q-6), (Q-7), (Q-8), (Q-9), (Q-10), (Q-11), (Q-12) and/or (Q-13)

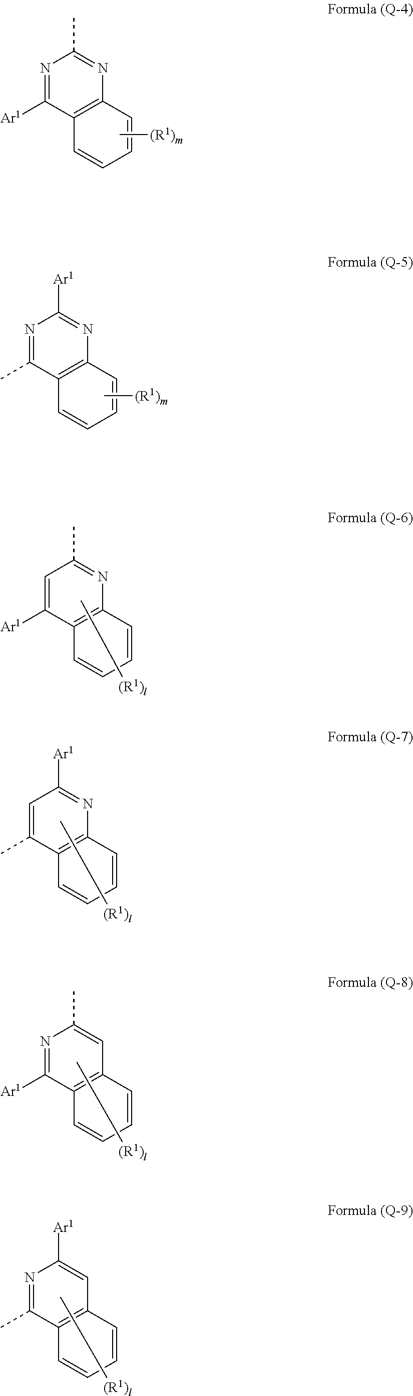

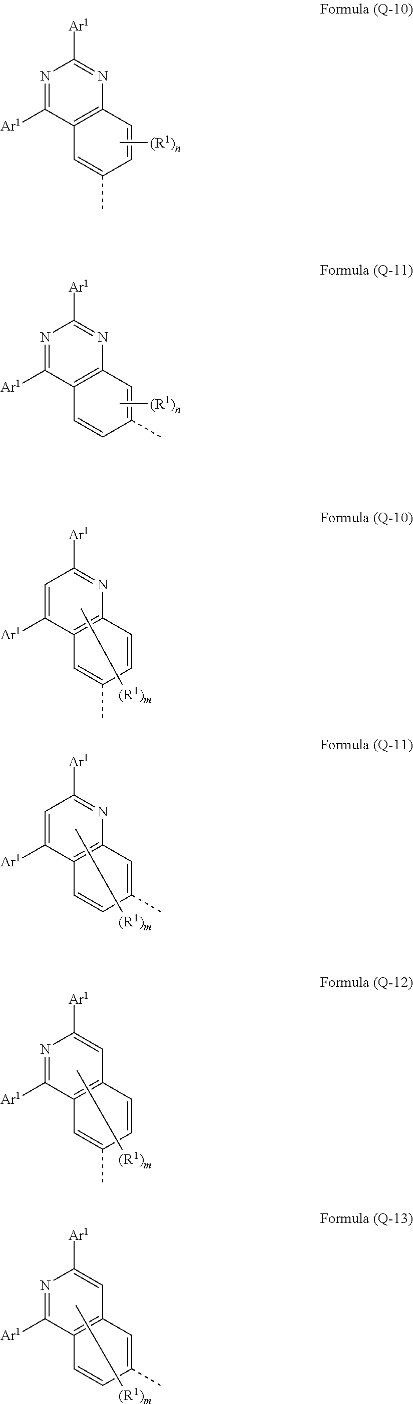

in which the symbols Ar¹ and R¹ have the definition detailed above, inter alia, for formula (I) and (Q-1), the dotted bond marks the attachment position and I is 1, 2, 3, 4 or 5, preferably 0, 1 or 2, m is 0, 1, 2, 3 or 4, preferably 0, 1 or 2, and n is 0, 1, 2 or 3, preferably 0 or 1.

It may further be the case that the Q¹ and/or Q² groups detailed, inter alia, in the formulae (I), (II), (Ia) and/or (IIa) are selected from structures of the

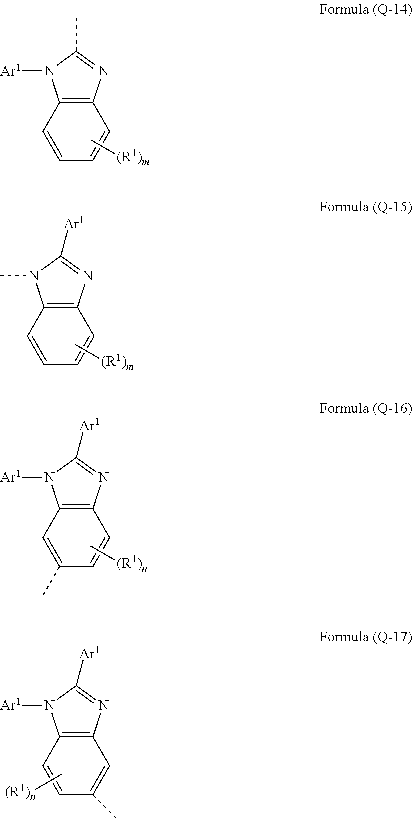

in which the symbols Ar¹ and R¹ have the definition detailed above, inter alia, for formula (I) and (Q-1), the dotted bond marks the attachment position and m is 0, 1, 2, 3 or 4, preferably 0, 1 or 2, and n is 0, 1, 2 or 3, preferably 0 or 1.

Preferably, the symbol Ar¹ represents an aryl or heteroaryl radical, such that an aromatic or heteroaromatic group of an aromatic or heteroaromatic ring system is bonded directly, i.e. via an atom of the aromatic or heteroaromatic group, to the respective atom of the further group, for example the carbon or nitrogen atom of the (Q-1) to (Q-17) groups shown above.

In a further preferred embodiment of the invention, Ar¹ is the same or different at each instance and is an aromatic or heteroaromatic ring system having 6 to 24 aromatic ring atoms, preferably 6 to 18 aromatic ring atoms, and is more preferably an aromatic ring system having 6 to 12 aromatic ring atoms or a heteroaromatic ring system which has 6 to 13 aromatic ring atoms and may be substituted in each case by one or more R² radicals, but is preferably unsubstituted, where R² may have the definition given above, especially in formula (I). Examples of suitable Ar¹ groups are selected from the group consisting of phenyl, ortho-, meta- or para-biphenyl, terphenyl, especially branched terphenyl, quaterphenyl, especially branched quaterphenyl, 1-, 2-, 3- or 4-fluorenyl, 1-, 2-, 3- or 4-spirobifluorenyl, pyridyl, pyrimidinyl, 1-, 2-, 3- or 4-dibenzofuranyl, 1-, 2-, 3- or 4-dibenzothienyl and 1-, 2-, 3- or 4-carbazolyl, each of which may be substituted by one or more R² radicals, but are preferably unsubstituted.

Advantageously, Ar¹ in the formulae (Q-1) to (Q-17) is an aromatic ring system which has 6 to 12 aromatic ring atoms and may be substituted by one or more R² radicals, but is preferably unsubstituted, where R² may have the definition given above, especially in formula (I).

Preferably, the R² radicals in the formulae (Q-1) to (Q-17) do not form a fused ring system with the ring atoms of the aryl group or heteroaryl group Ar¹ to which the R² radicals are bonded. This includes the formation of a fused ring system with possible R³ substituents which may be bonded to the R² radicals.

In addition, compounds of formulae (I), (II), (Ia) and/or (IIa) show surprising advantages where the Q¹ and/or Q² group is selected from structures of the formulae (Q-18), (Q-19), (Q-20), (Q-21), (Q-22), (Q-23), (Q-24), (Q-25), (Q-26), (Q-27) and/or (Q-28)

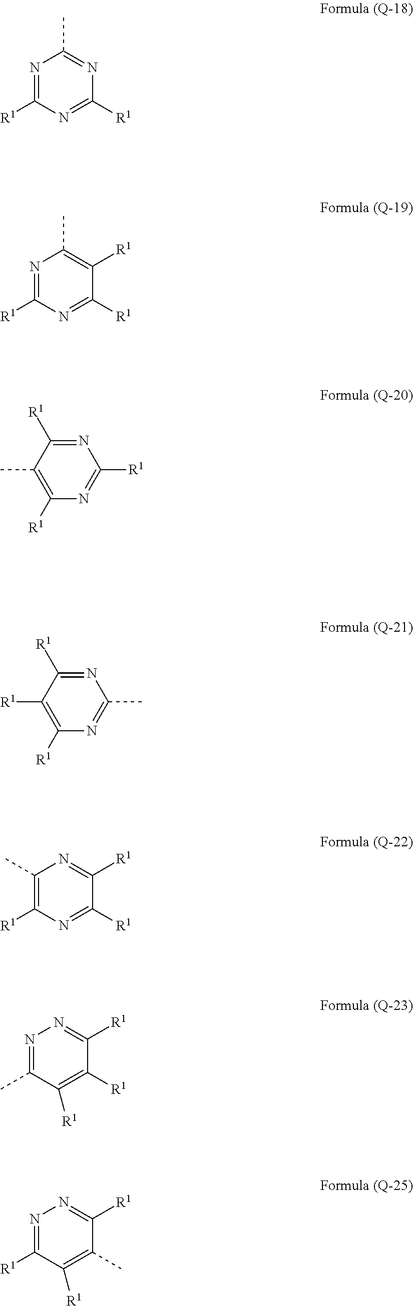

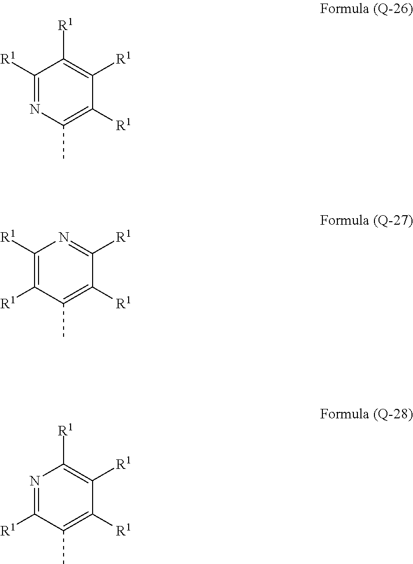

in which the R¹ symbols have the definition detailed above, especially for formula (I), and the dotted bond marks the attachment position.

In a preferred embodiment, in the aforementioned formulae, especially the formulae (I), (Ia), (II) and/or (IIa), the Q¹ group and the Q² group are selected from groups of the formulae (Q-1) to (Q-13).

In a further configuration, in the aforementioned formulae, especially the formulae (I), (Ia), (II) and/or (IIa), the Q¹ group and the Q² group are selected from groups of the formulae (Q-14) to (Q-17).

It may further be the case that, in the aforementioned formulae, especially the formulae (I), (Ia), (II) and/or (IIa), the Q¹ group and the Q² group are selected from groups of the formulae (Q-18) to (Q-28).

In addition, in the aforementioned formulae, especially the formulae (I), (Ia), (II) and/or (IIa), one of the Q¹, Q² groups may be selected from groups of the formulae (Q-1) to (Q-13) and one of the Q, Q² groups may be selected from groups of the formulae (Q-14) to (Q-17).

It may further be the case that, in the aforementioned formulae, especially the formulae (I), (Ia), (II) and/or (IIa), one of the Q¹, Q² groups is selected from groups of the formulae (Q-1) to (Q-13) and one of the Q¹, Q² groups is selected from groups of the formulae (Q-18) to (Q-28).

It may additionally be the case that, in the aforementioned formulae, especially the formulae (I), (Ia), (II) and/or (IIa), one of the Q¹, Q² groups is selected from groups of the formulae (Q-14) to (Q-17) and one of the Q¹, Q² groups is selected from groups of the formulae (Q-18) to (Q-28).

In a further configuration, the electron-transporting group Q¹ and Q² in the aforementioned formulae, especially the formulae (I), (Ia), (II) and/or (IIa), is the same.

It may further be the case that the electron-transporting group Q¹ and Q² in the aforementioned formulae, especially the formulae (I), (Ia), (II) and/or (IIa), is not the same.

When X is CR¹ or when the aromatic and/or heteroaromatic groups are substituted by R¹ substituents, these R¹ substituents are preferably selected from the group consisting of H, D, F, CN, N(Ar¹)₂, C(═O)Ar¹, P(═O)(Ar¹)₂, a straight-chain alkyl or alkoxy group having 1 to 10 carbon atoms or a branched or cyclic alkyl or alkoxy group having 3 to 10 carbon atoms or an alkenyl group having 2 to 10 carbon atoms, each of which may be substituted by one or more R² radicals, where one or more nonadjacent CH₂ groups may be replaced by O and where one or more hydrogen atoms may be replaced by D or F, an aromatic or heteroaromatic ring system which has 5 to 24 aromatic ring atoms and may be substituted in each case by one or more R² radicals, but is preferably unsubstituted, or an aralkyl or heteroaralkyl group which has 5 to 25 aromatic ring atoms and may be substituted by one or more R² radicals; at the same time, it is optionally possible for two R¹ substituents bonded to the same carbon atom or to adjacent carbon atoms to form a monocyclic or polycyclic, aliphatic, aromatic or heteroaromatic ring system which may be substituted by one or more R¹ radicals. The Ar¹ group may have the definition given above, especially for structure (Q-1). Preferably, the symbol Ar¹ represents an aryl or heteroaryl radical, such that an aromatic or heteroaromatic group of an aromatic or heteroaromatic ring system is bonded directly, i.e. via an atom of the aromatic or heteroaromatic group, to the respective atom of the further group, for example the carbon, nitrogen or phosphorus atom of the N(Ar¹)₂, C(═O)Ar¹, P(═O)(Ar¹)₂ groups.

More preferably, these R¹ substituents are selected from the group consisting of H, D, F, CN, N(Ar¹)₂, a straight-chain alkyl group having 1 to 8 carbon atoms, preferably having 1, 2, 3 or 4 carbon atoms, or a branched or cyclic alkyl group having 3 to 8 carbon atoms, preferably having 3 or 4 carbon atoms, or an alkenyl group having 2 to 8 carbon atoms, preferably having 2, 3 or 4 carbon atoms, each of which may be substituted by one or more R² radicals, but is preferably unsubstituted, or an aromatic or heteroaromatic ring system which has 6 to 24 aromatic ring atoms, preferably 6 to 18 aromatic ring atoms, more preferably 6 to 13 aromatic ring atoms, and may be substituted in each case by one or more nonaromatic R¹ radicals, but is preferably unsubstituted; at the same time, it is optionally possible for two R¹ substituents bonded to the same carbon atom or to adjacent carbon atoms to form a monocyclic or polycyclic aliphatic ring system which may be substituted by one or more R² radicals, but is preferably unsubstituted. The Ar¹ group may have the definition given above, especially for structure (Q-1). Preferably, the symbol Ar¹ represents an aryl or heteroaryl radical, such that an aromatic or heteroaromatic group of an aromatic or heteroaromatic ring system is bonded directly, i.e. via an atom of the aromatic or heteroaromatic group, to the respective atom of the further group, for example the nitrogen atom of the N(Ar¹)₂ group.

Most preferably, the R¹ substituents are selected from the group consisting of H and an aromatic or heteroaromatic ring system which has 6 to 18 aromatic ring atoms, preferably 6 to 13 aromatic ring atoms, and may be substituted in each case by one or more nonaromatic R² radicals, but is preferably unsubstituted. Examples of suitable R¹ substituents are selected from the group consisting of phenyl, ortho-, meta- or para-biphenyl, terphenyl, especially branched terphenyl, quaterphenyl, especially branched quaterphenyl, 1-, 2-, 3- or 4-fluorenyl, 1-, 2-, 3- or 4-spirobifluorenyl, pyridyl, pyrimidinyl, 1-, 2-, 3- or 4-dibenzofuranyl, 1-, 2-, 3- or 4-dibenzothienyl and 1-, 2-, 3- or 4-carbazolyl, each of which may be substituted by one or more R² radicals, but are preferably unsubstituted.

It may additionally be the case that, in a structure of formula (I), (Ia), (II), (IIa) and/or (Q-1) to (Q-28), at least one R¹ and/or Ar¹ radical is a group selected from the formulae (R¹-1) to (R¹-79)

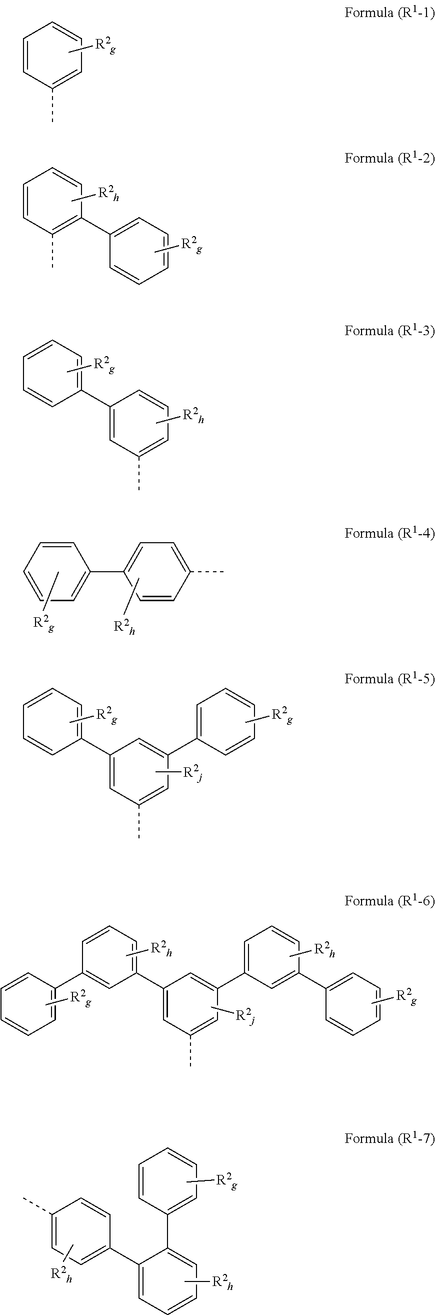

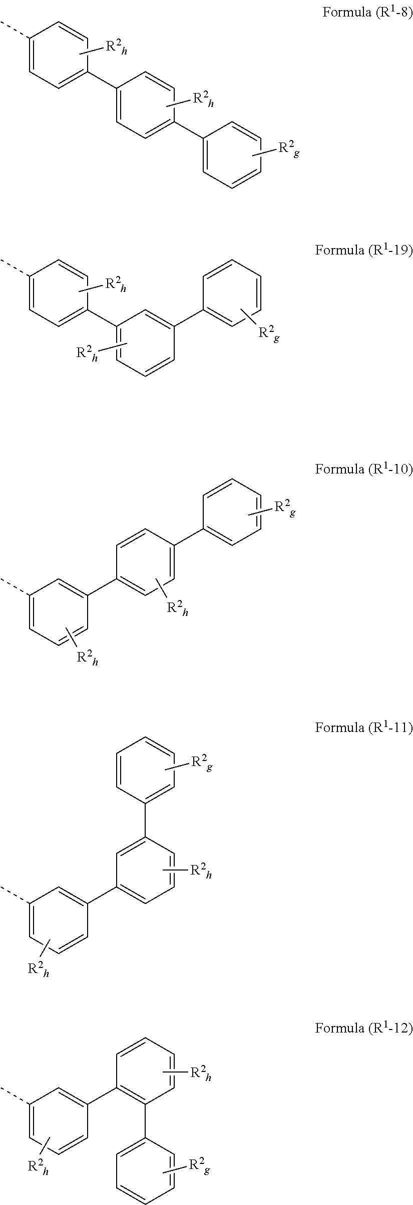

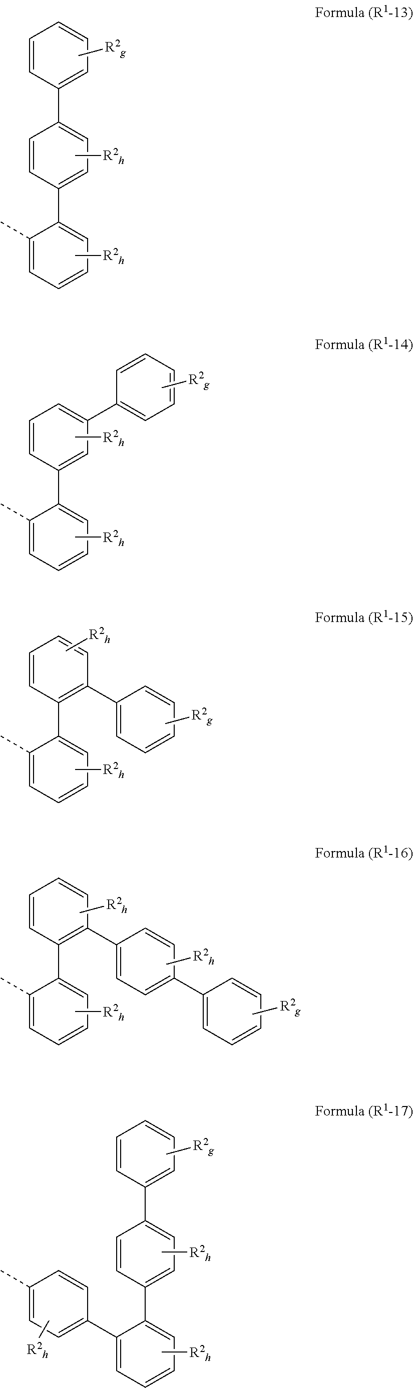

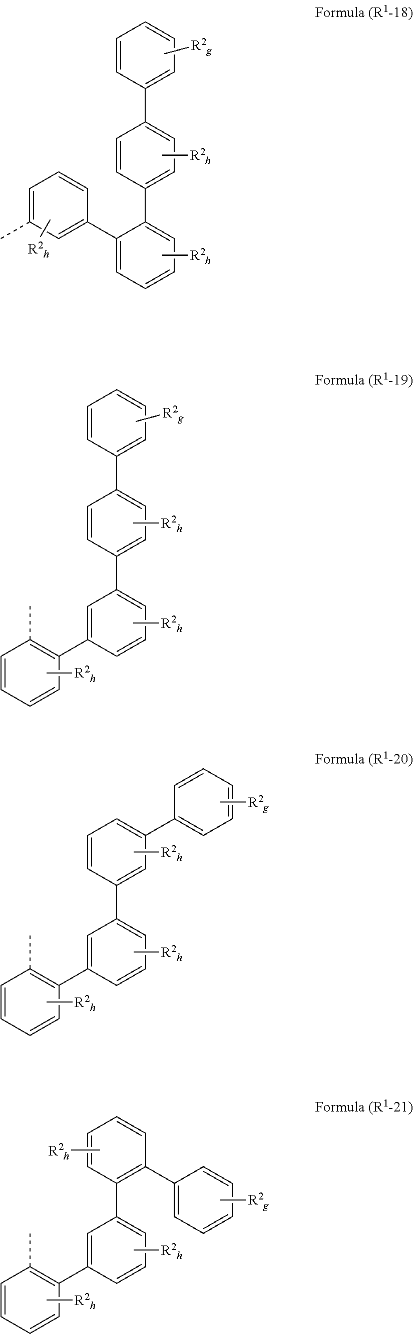

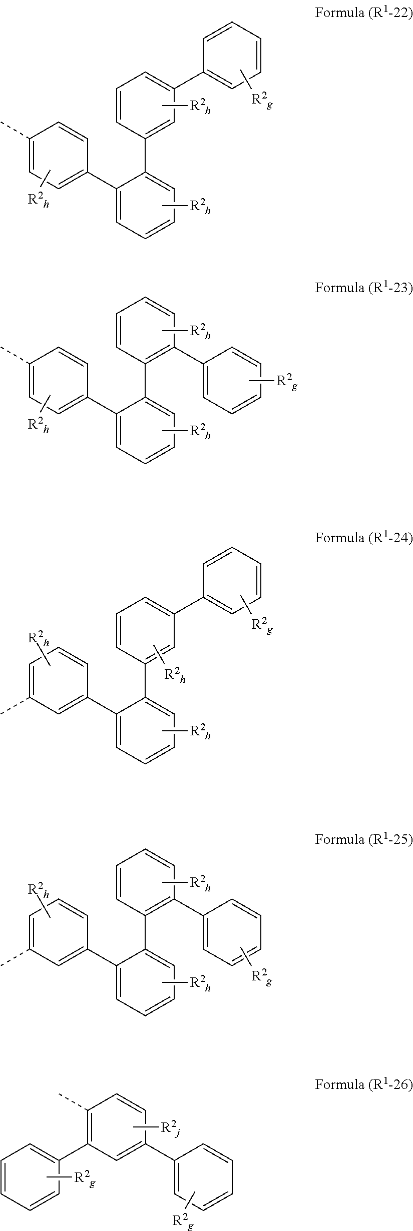

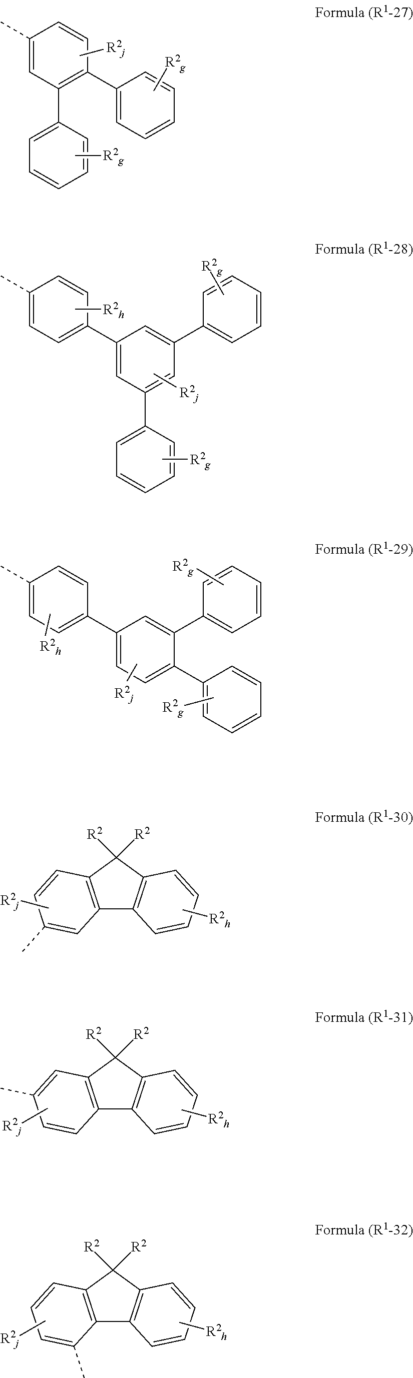

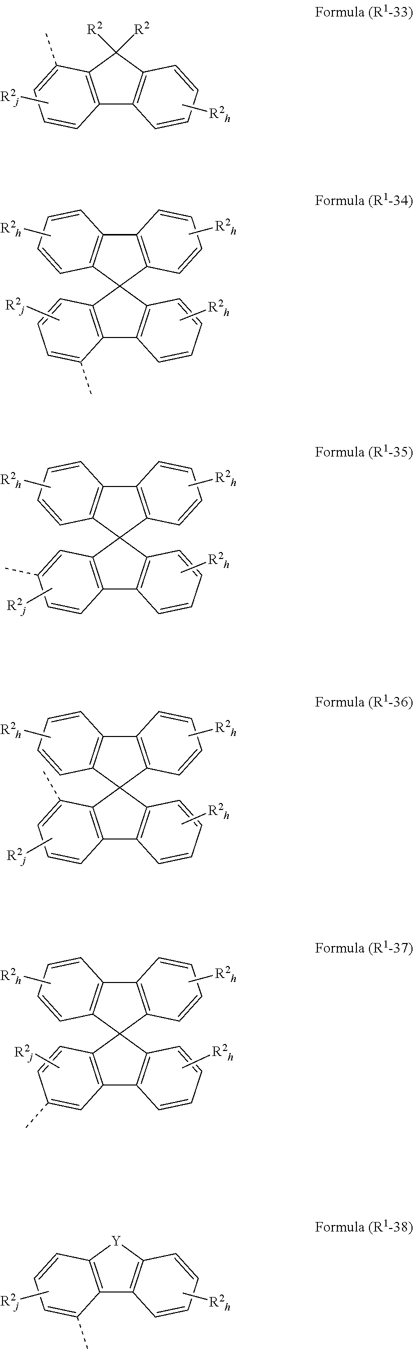

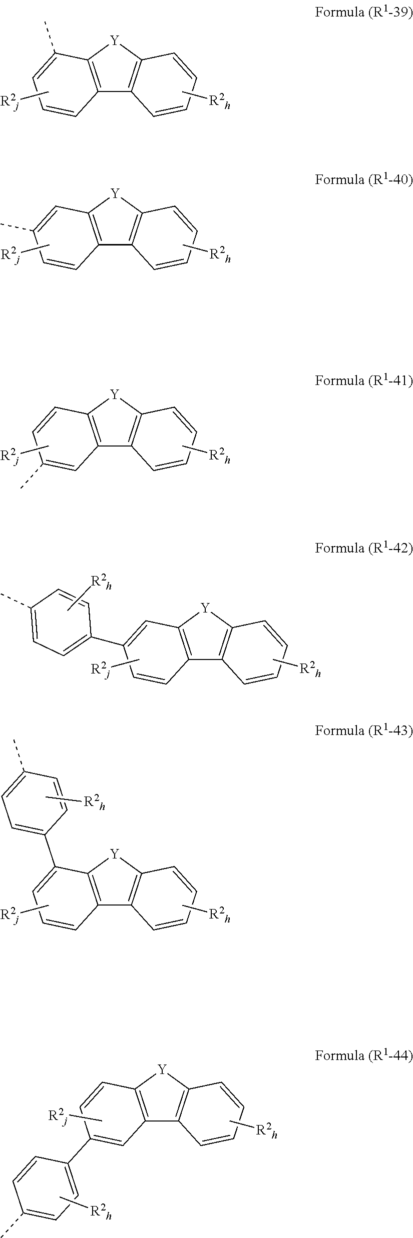

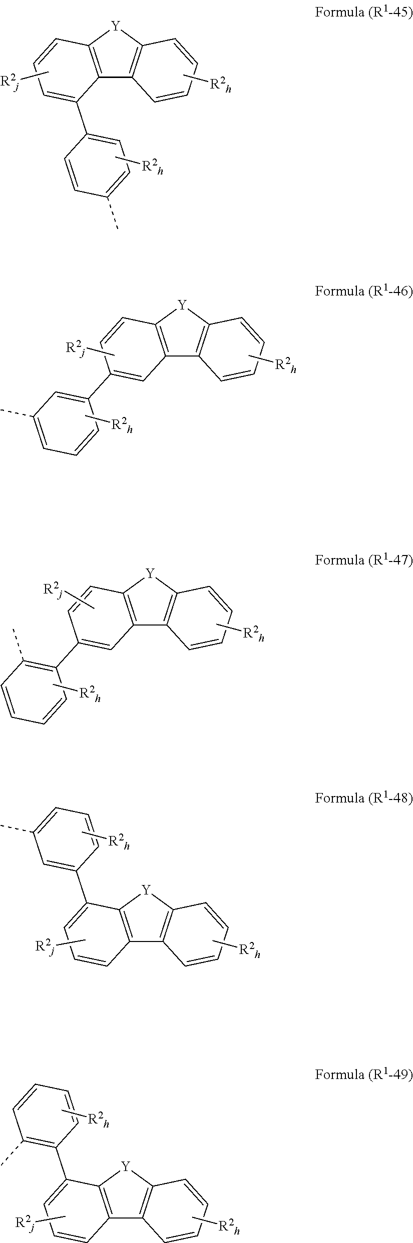

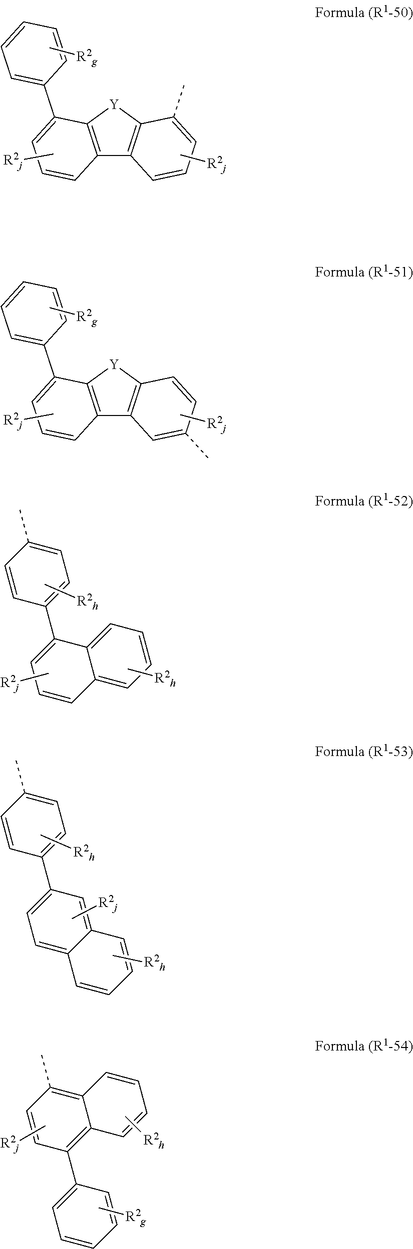

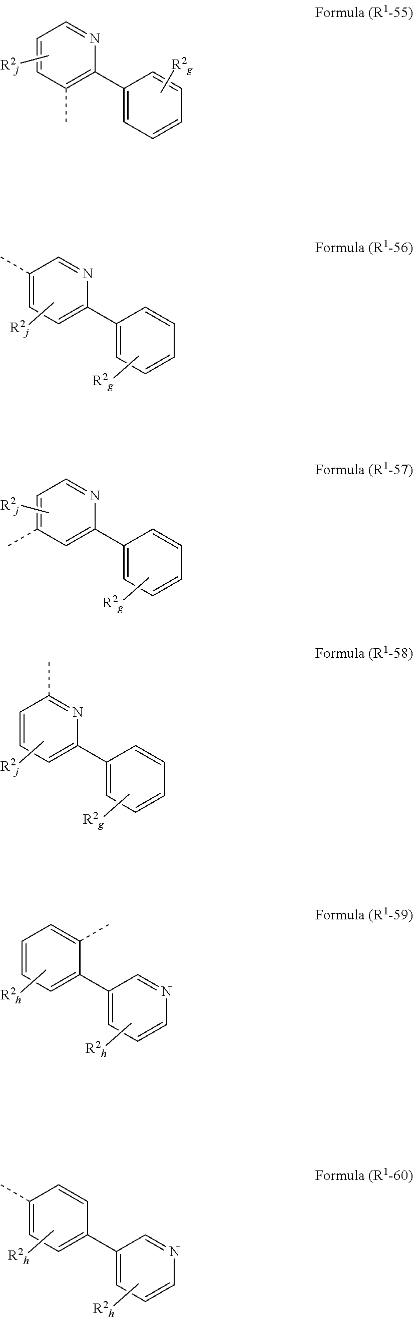

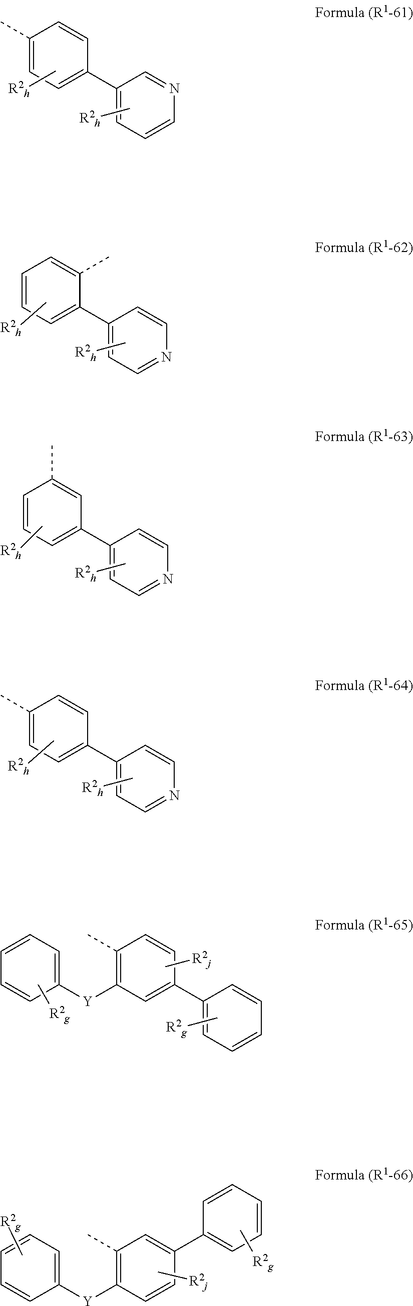

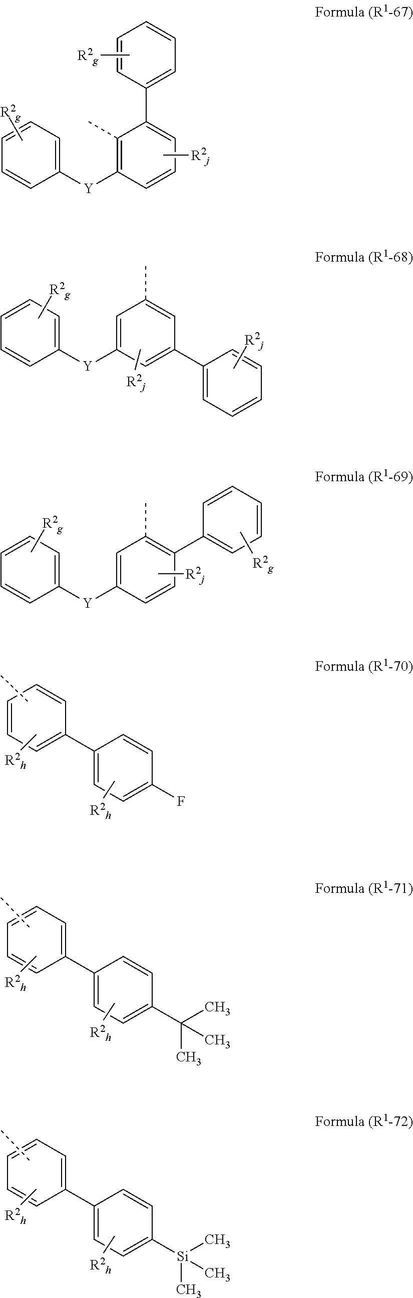

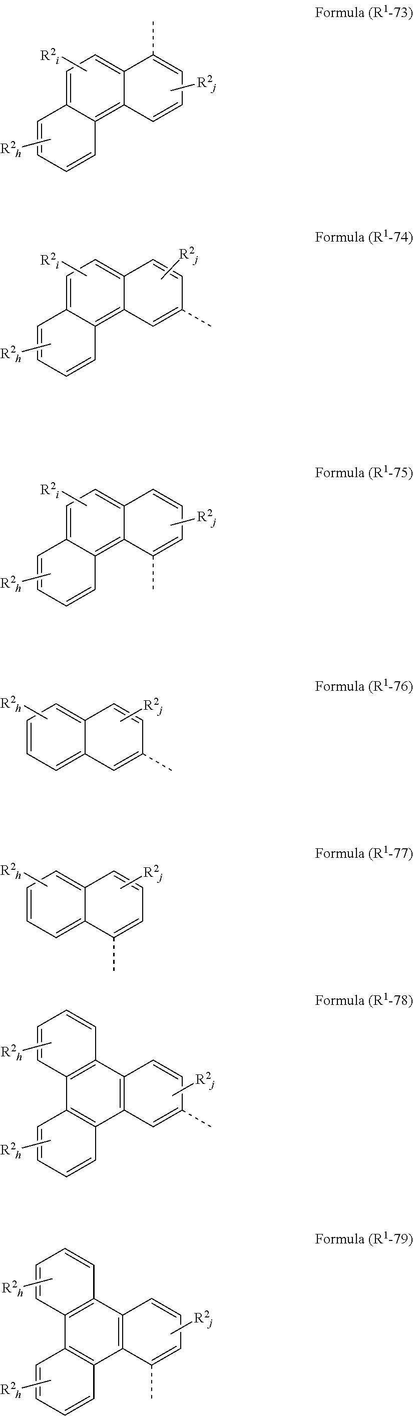

where the symbols used are as follows:
Y is O, S or NR², preferably O or S;
i at each instance is independently 0, 1 or 2;
j at each instance is independently 0, 1, 2 or 3;
h at each instance is independently 0, 1, 2, 3 or 4;
g at each instance is independently 0, 1, 2, 3, 4 or 5;
R² may have the definition given above, especially for formula (I), and
the dotted bond marks the attachment position.

It may preferably be the case that the sum total of the indices g, h, i and j in the structures of the formula (R¹-1) to (R¹-79) is not more than 3 in each case, preferably not more than 2 and more preferably not more than 1.

Preferably, the L¹ and/or L² group may form through-conjugation with the electron-transporting group Q¹ and/or Q² and the dibenzofuran structure (Y ═O) and/or the dibenzothiophene structure (Y═S) of the formula (I), (Ia), (II) and/or (IIa). Through-conjugation of the aromatic or heteroaromatic systems is formed as soon as direct bonds are formed between adjacent aromatic or heteroaromatic rings. A further bond between the aforementioned conjugated groups, for example via a sulphur, nitrogen or oxygen atom or a carbonyl group, is not detrimental to conjugation. In the case of a fluorene system, the two aromatic rings are bonded directly, where the sp³-hybridized carbon atom in position 9 does prevent fusion of these rings, but conjugation is possible, since this sp³-hybridized carbon atom in position 9 does not necessarily lie between the electron-transporting group Q¹ and/or Q² and the dibenzofuran structure (Y═O) and/or the dibenzothiophene structure (Y═S). In contrast, in the case of a spirobifluorene structure, through-conjugation can be formed if the bond between the electron-transporting group Q¹ and/or Q² and the dibenzofuran structure (Y═O) and/or the dibenzothiophene structure (Y═S) of the formula (I), (Ia), (II) and/or (IIa) is via the same phenyl group in the spirobifluorene structure or via phenyl groups in the spirobifluorene structure that are bonded directly to one another and are in one plane. If the bond between the electron-transporting group Q¹ and/or Q² and the dibenzofuran structure (Y═O) and/or the dibenzothiophene structure (Y═S) of the formula (I), (Ia), (II) and/or (IIa) is via different phenyl groups in the spirobifluorene structure bonded via the sp³-hybridized carbon atom in position 9, the conjugation is interrupted.

In a further preferred embodiment of the invention, L¹ and/or L² is the same or different at each instance and is a single bond or an aromatic or heteroaromatic ring system which has 5 to 24 aromatic ring atoms and may be substituted by one or more R² radicals. More preferably, L¹ and/or L² is the same or different at each instance and is a single bond or an aromatic ring system having 6 to 12 aromatic ring atoms or a heteroaromatic ring system which has 6 to 13 aromatic ring atoms and may be substituted in each case by one or more R² radicals, but is preferably unsubstituted, where R² may have the definition given above, especially for formula (I). Further preferably, the symbol L¹ and/or L² is the same or different at each instance and is a single bond or an aryl or heteroaryl radical, such that an aromatic or heteroaromatic group of an aromatic or heteroaromatic ring system is bonded directly, i.e. via an atom in the aromatic or heteroaromatic group, to the respective atom in the other group. Most preferably, L¹ and/or L² is a single bond. Examples of suitable aromatic or heteroaromatic ring systems L¹ and/or L² are selected from the group consisting of ortho-, meta- or para-phenylene, biphenyl, fluorene, pyridine, pyrimidine, triazine, dibenzofuran and dibenzothiophene, each of which may be substituted by one or more R² radicals, but are preferably unsubstituted.

Preference is given to compounds comprising structures of the formulae (I), (II), (Ia) and/or (IIa) in which at least one L¹ and/or L² group of formula (I), (IIa) and/or (IIb) is a bond or a group selected from the formulae (L-1) to (L-70)

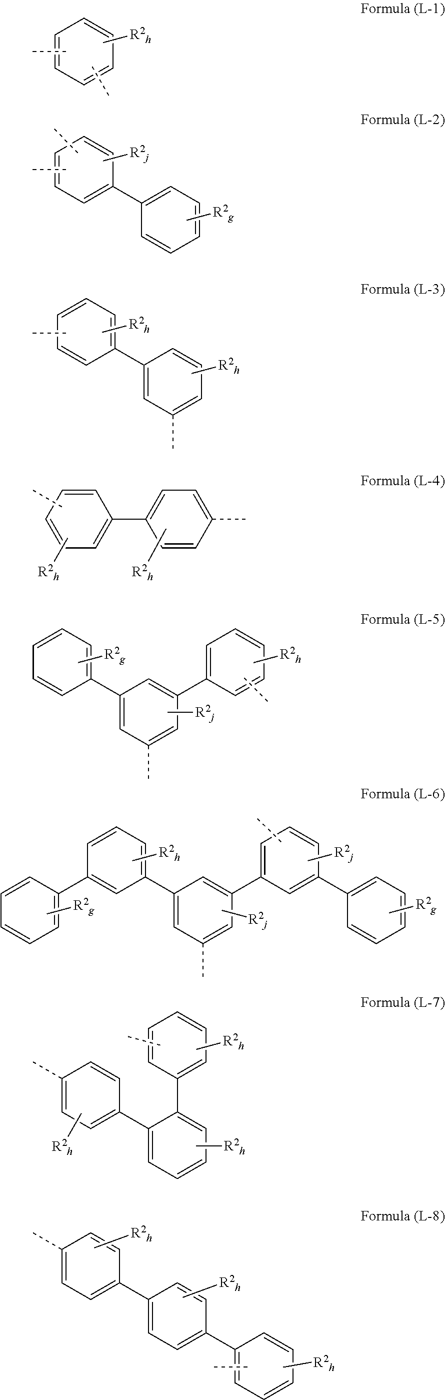

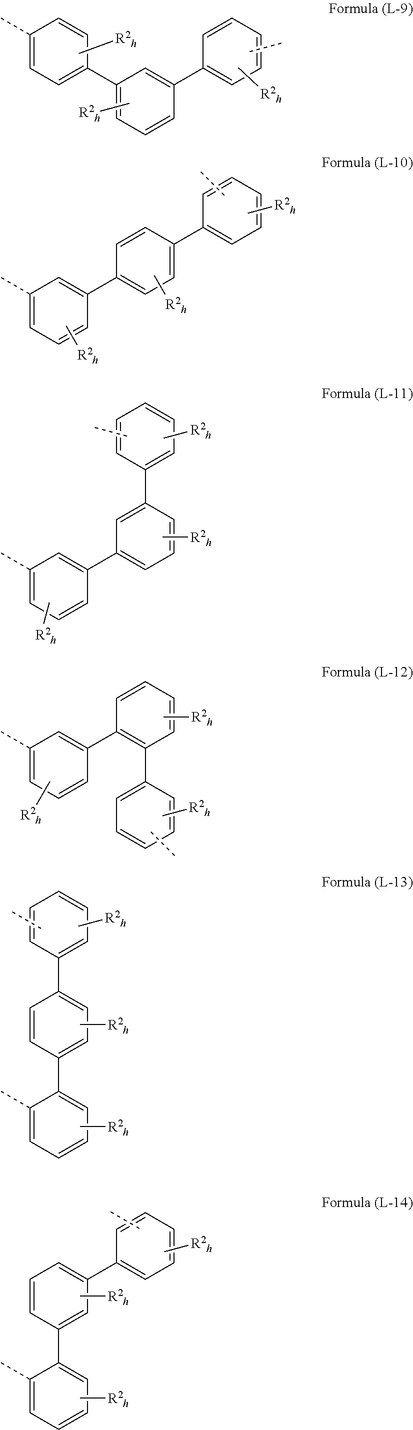

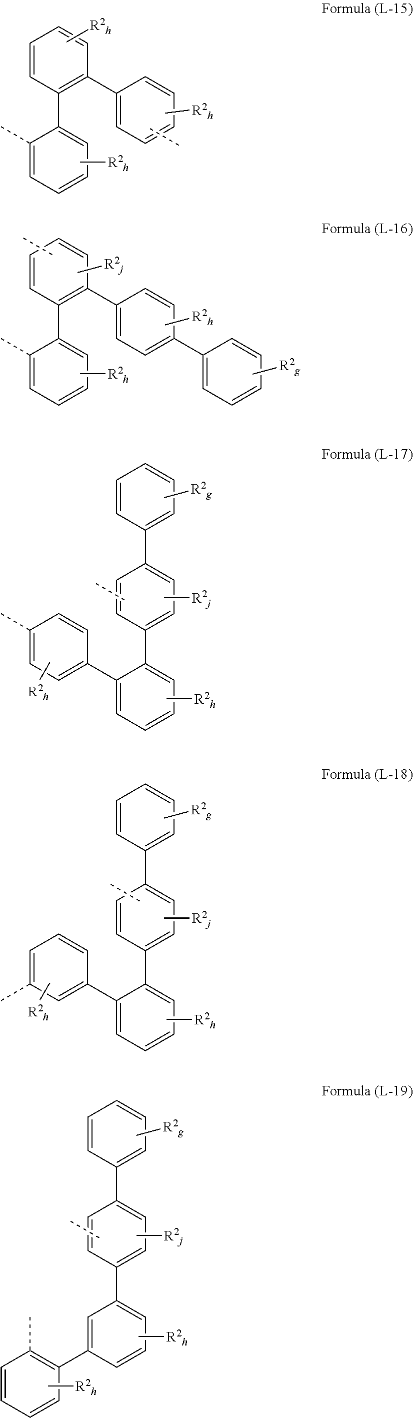

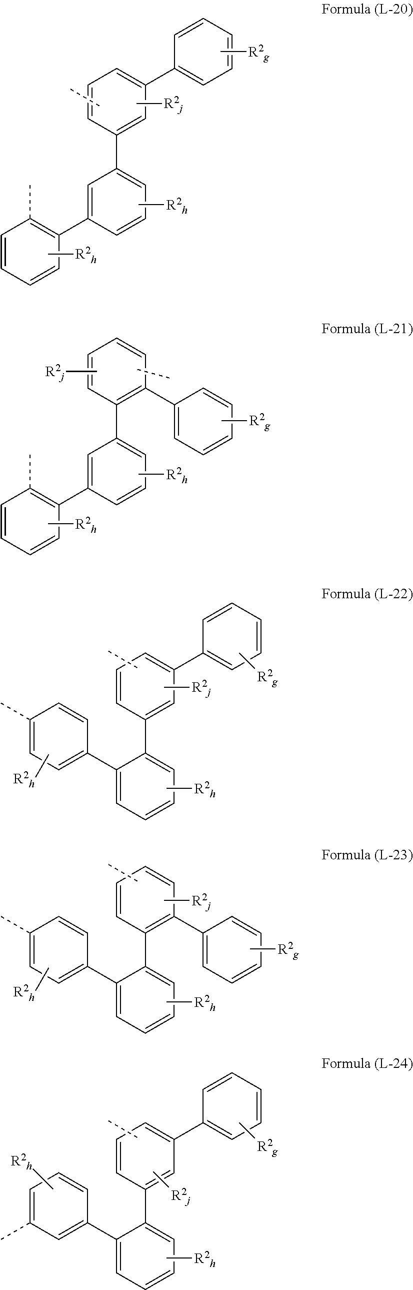

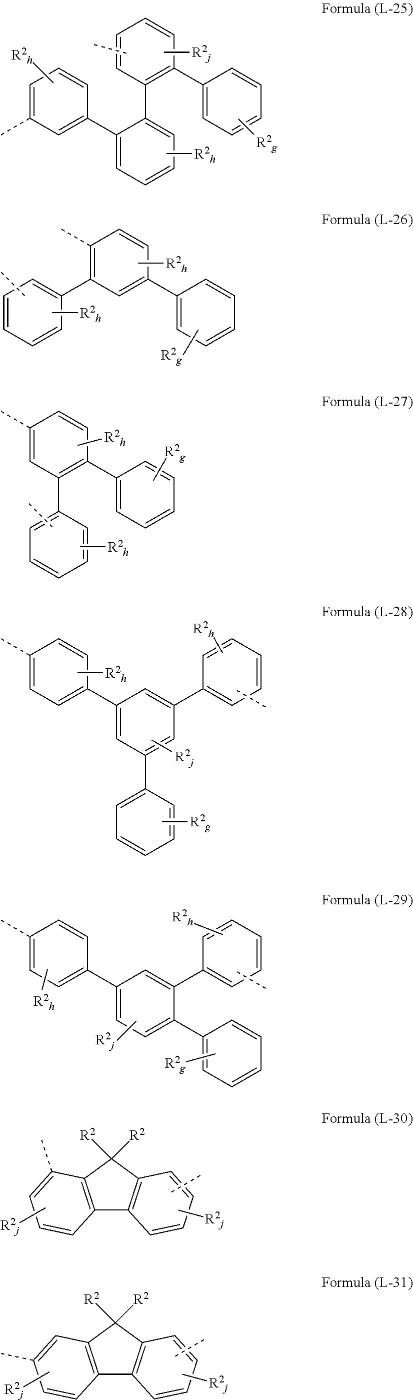

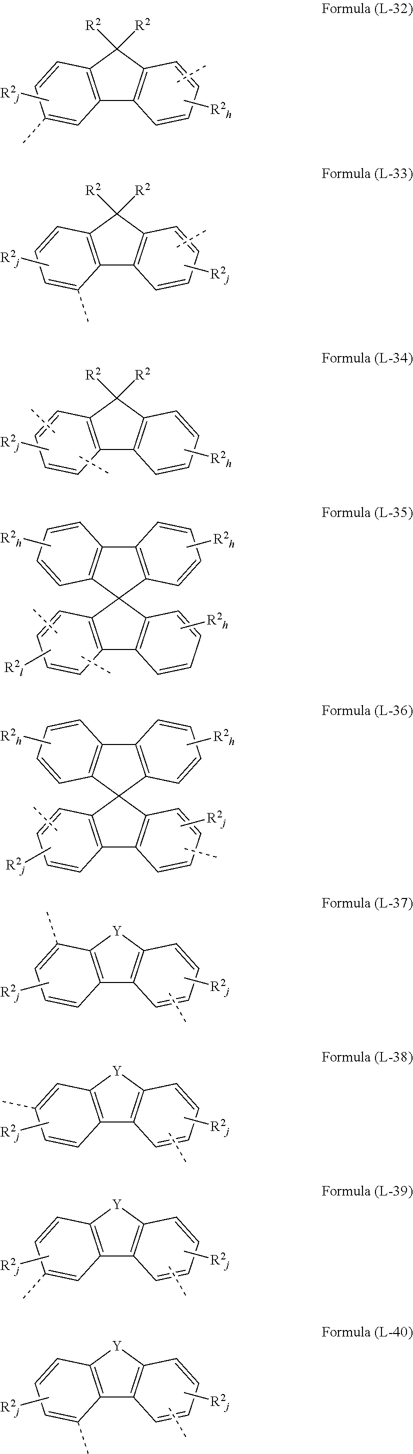

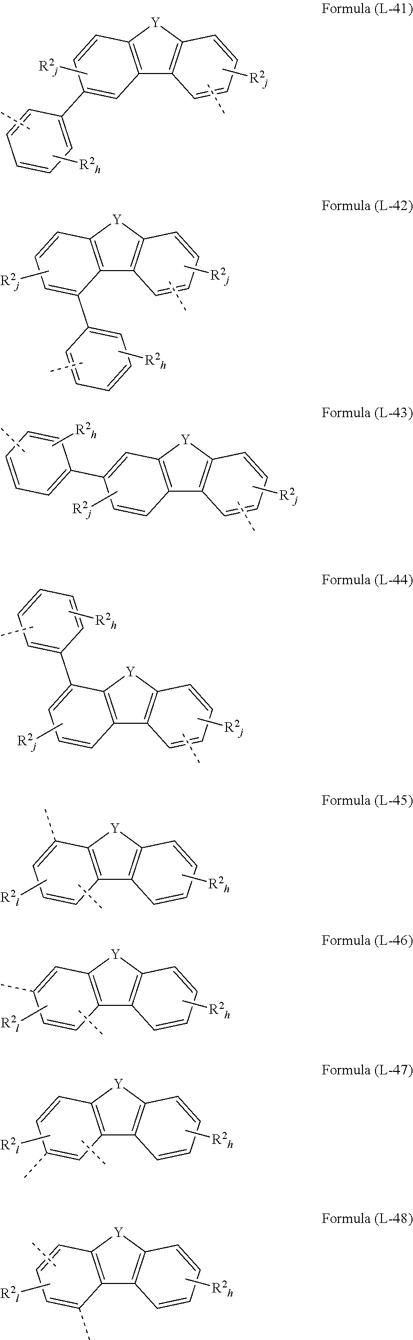

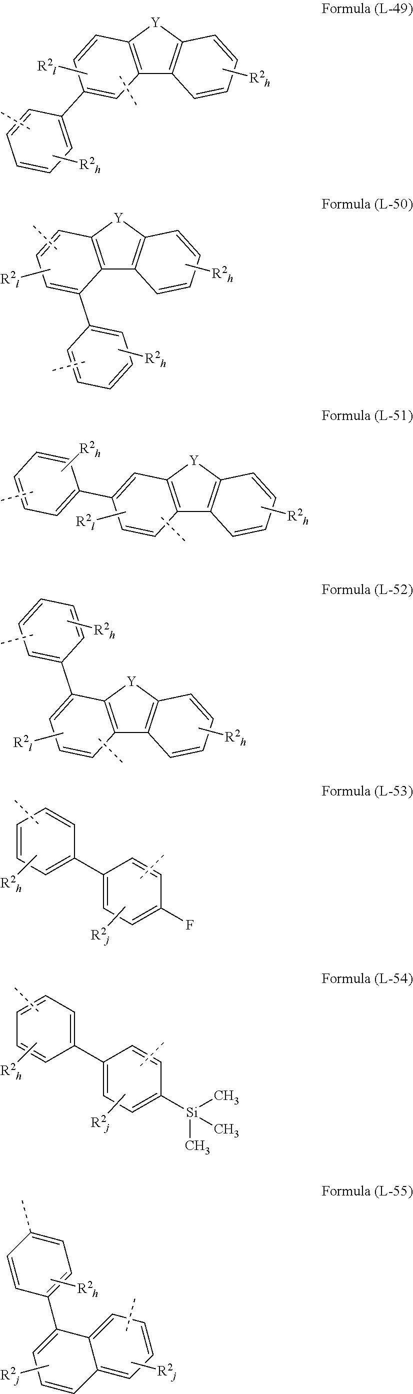

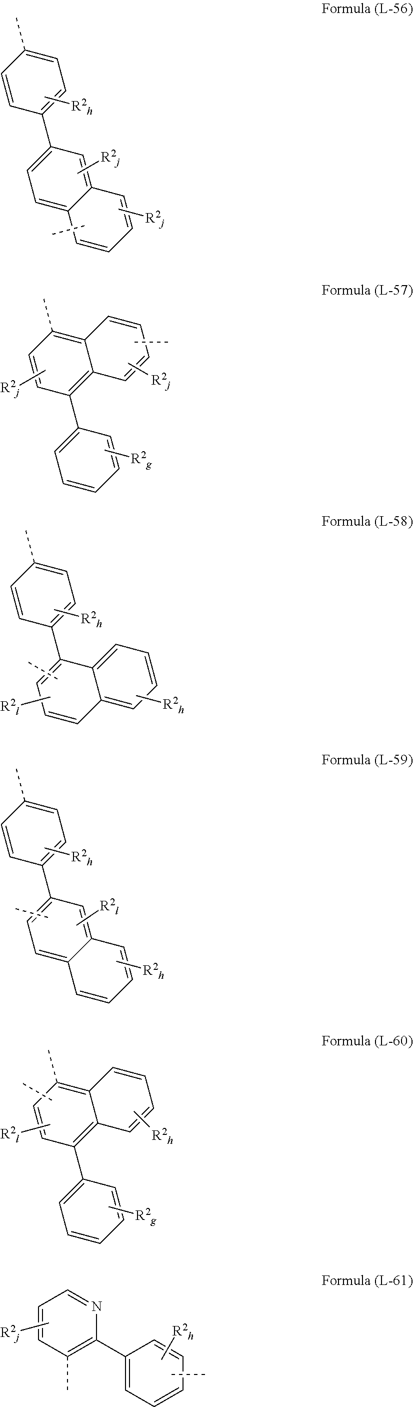

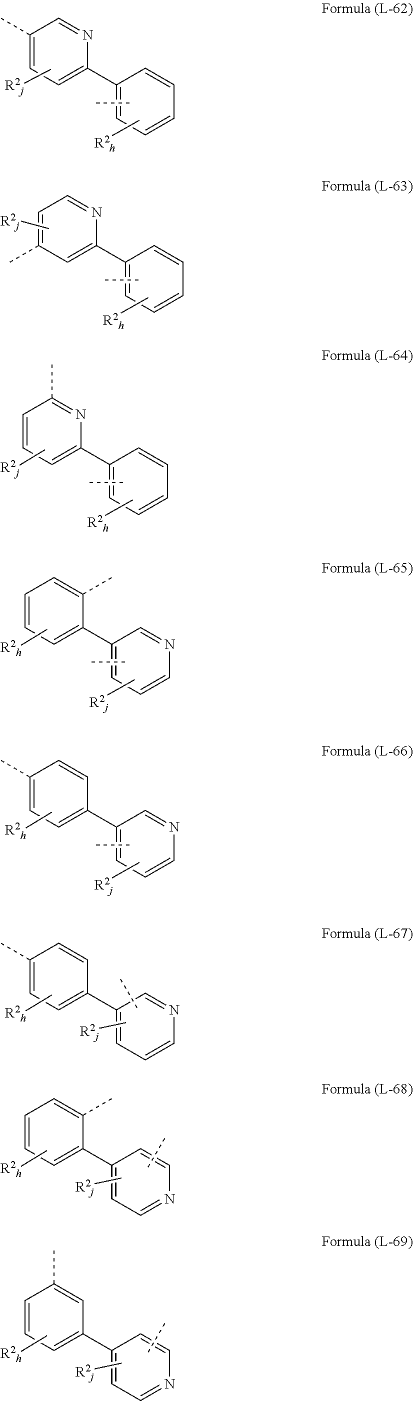

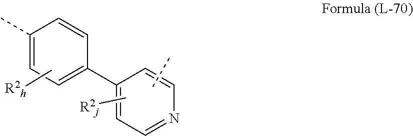

where the dotted bonds in each case mark the attachment positions, the index I is 0, 1 or 2, the index g is 0, 1, 2, 3, 4 or 5, j independently at each instance is 0, 1, 2 or 3; h independently at each instance is 0, 1, 2, 3 or 4; Y is O, S or NR², preferably O or S; and R² has the definition given above, especially for formula (I).

It may preferably be the case that the sum total of the indices I, g, h and j in the structures of the formula (L-1) to (L-70) is at most 3 in each case, preferably at most 2 and more preferably at most 1.

Advantageously, it may be the case that a compound of the invention comprising at least one structure of formula (I), (Ia), (II) and/or (IIa) does not comprise any carbazole and/or triarylamine group. More preferably, a compound of the invention does not comprise any hole-transporting group. Hole-transporting groups are known in the specialist field, these groups in many cases being carbazole, indenocarbazole, indolocarbazole, arylamine or diarylamine structures.

In a further preferred embodiment of the invention, R² is the same or different at each instance and is selected from the group consisting of H, D, F, CN, an aliphatic hydrocarbyl radical having 1 to 10 carbon atoms, preferably having 1, 2, 3 or 4 carbon atoms, or an aromatic or heteroaromatic ring system having 5 to 30 aromatic ring atoms, preferably having 5 to 24 aromatic ring atoms, more preferably having 5 to 13 aromatic ring atoms, which may be substituted by one or more alkyl groups each having 1 to 4 carbon atoms, but is preferably unsubstituted.

When the compound of the invention is substituted by aromatic or heteroaromatic R¹ or R² or Ar¹ groups, it is preferable when these do not have any aryl or heteroaryl groups having more than two aromatic six-membered rings fused directly to one another. More preferably, the substituents do not have any aryl or heteroaryl groups having six-membered rings fused directly to one another at all. The reason for this preference is the low triplet energy of such structures. Fused aryl groups which have more than two aromatic six-membered rings fused directly to one another but are nevertheless also suitable in accordance with the invention are phenanthrene and triphenylene, since these also have a high triplet level.

Examples of suitable compounds of the invention are the structures of the following formulae 1 to 111 shown below:

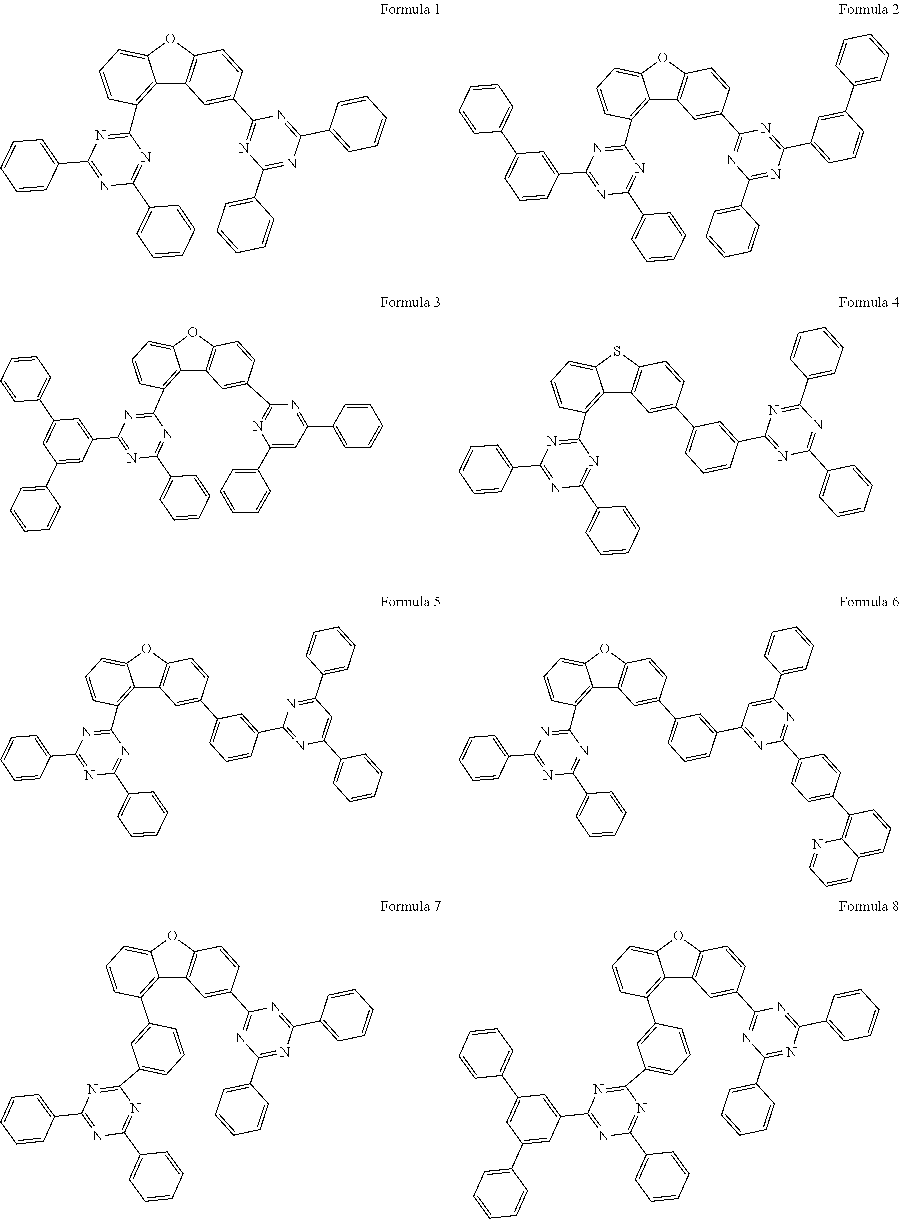

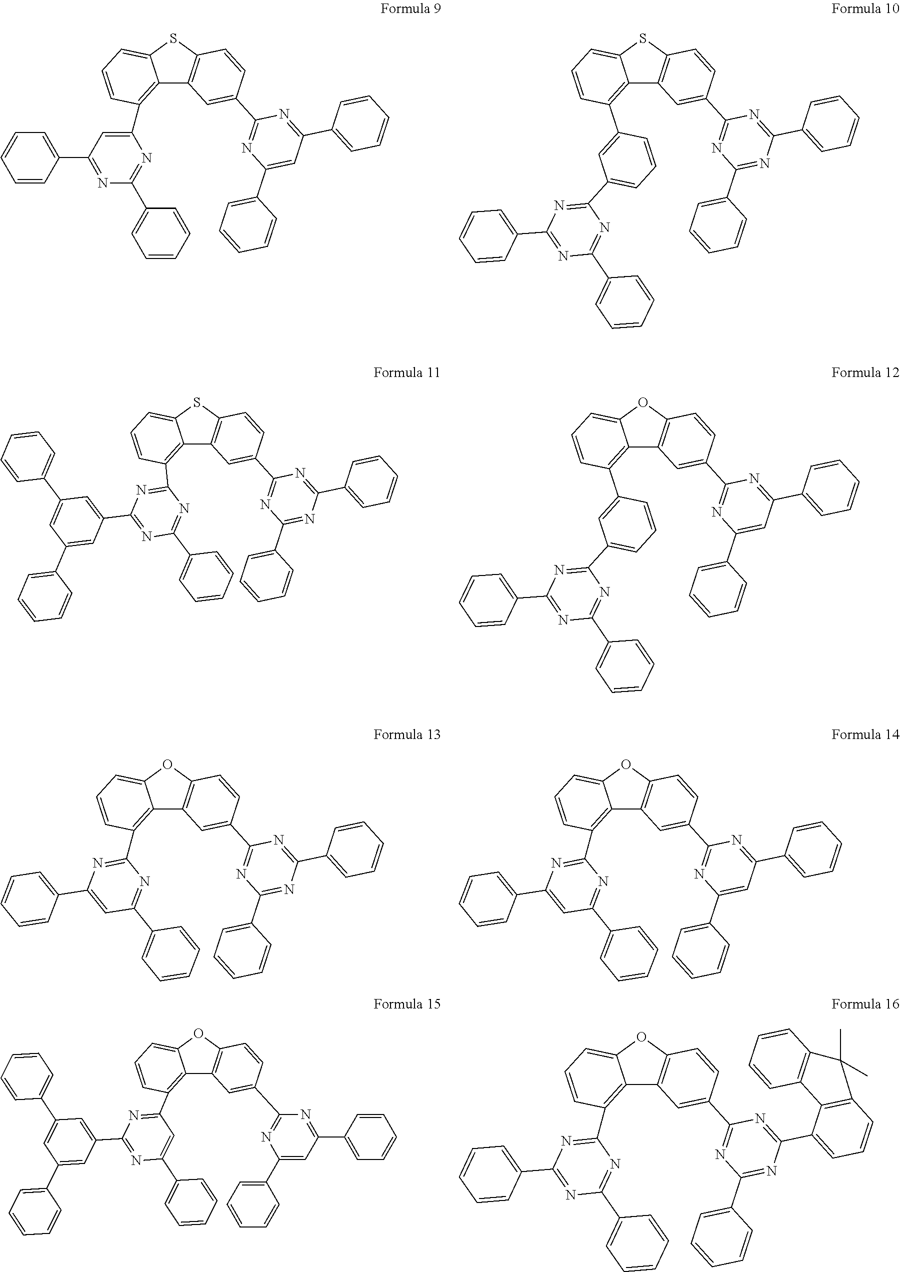

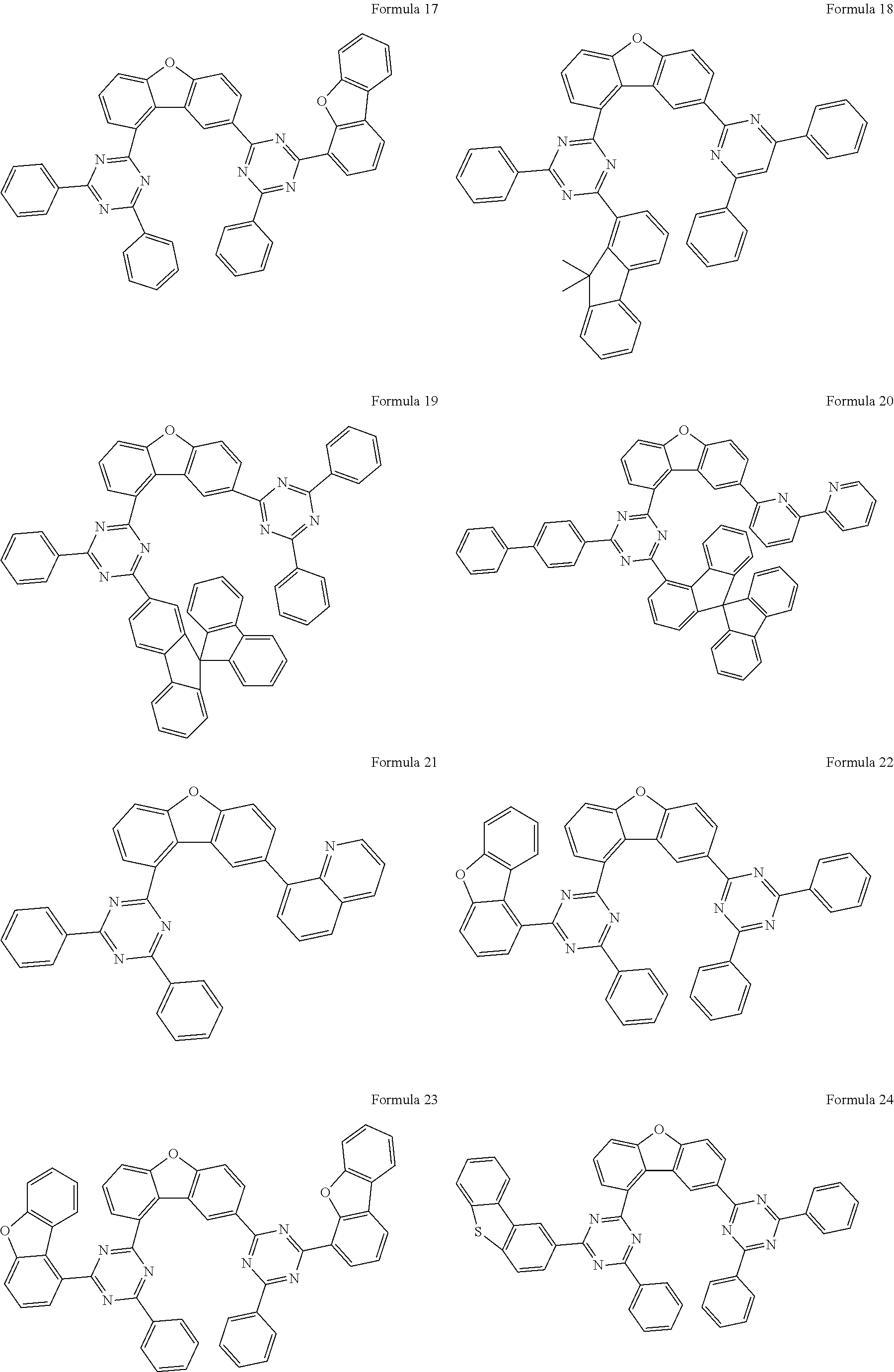

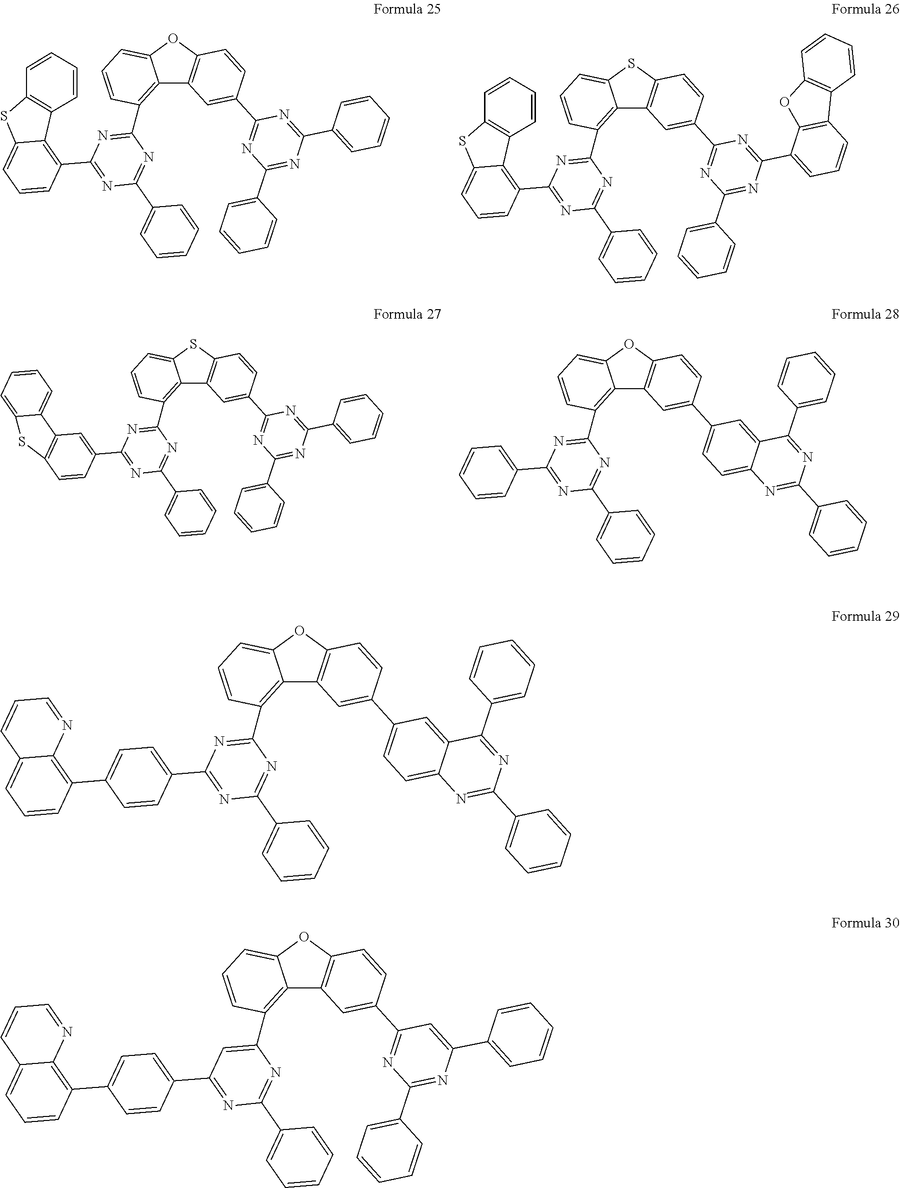

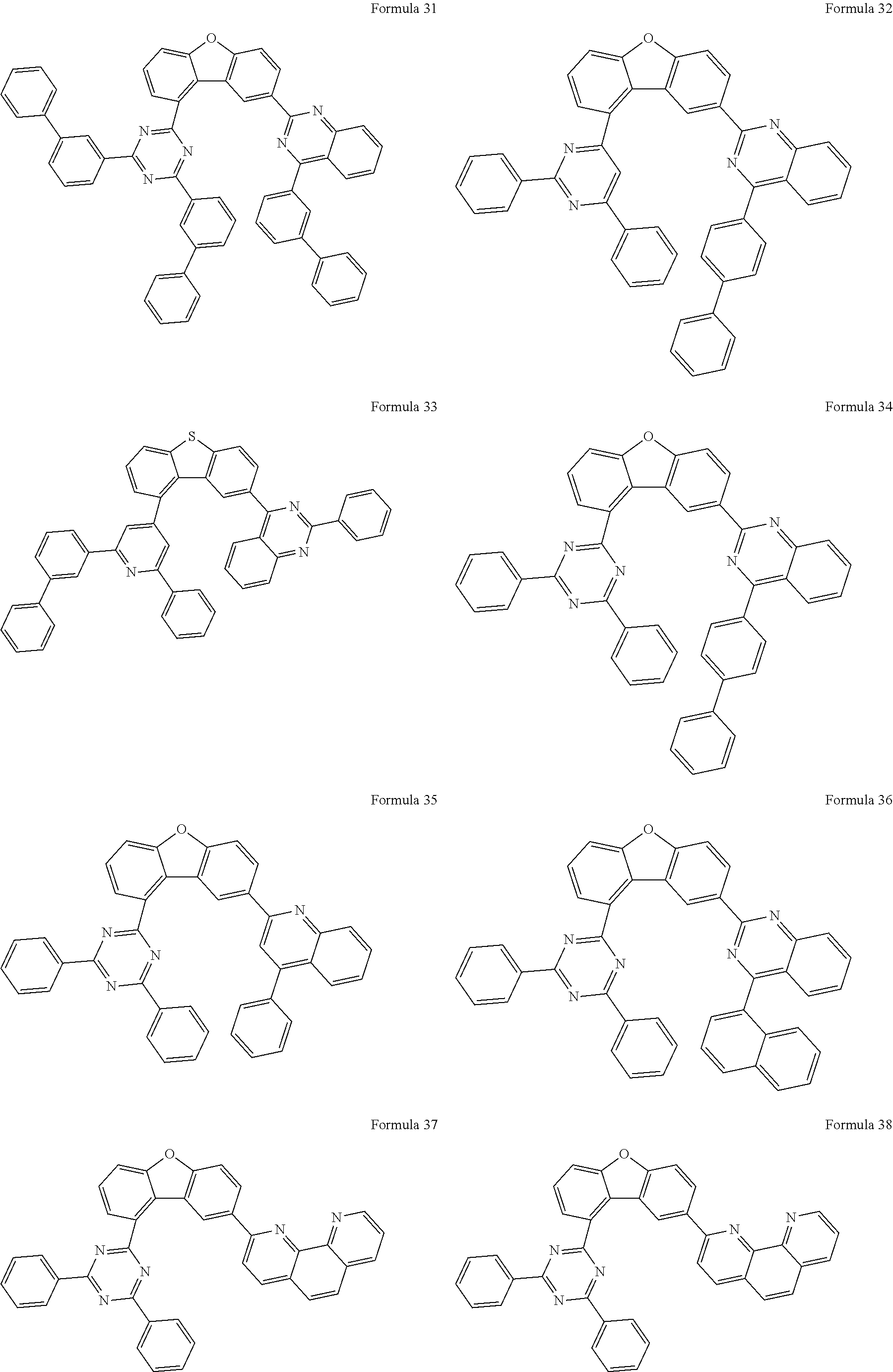

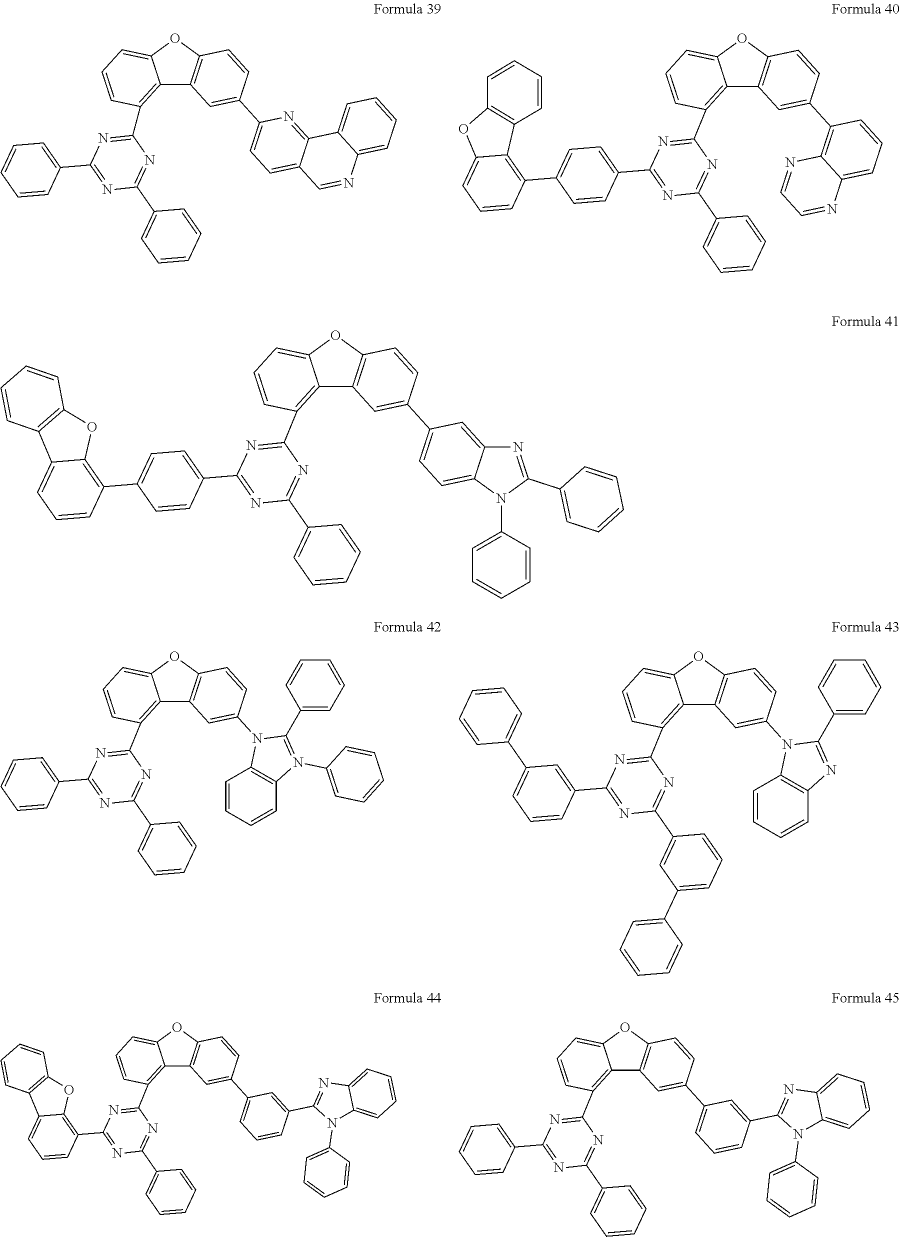

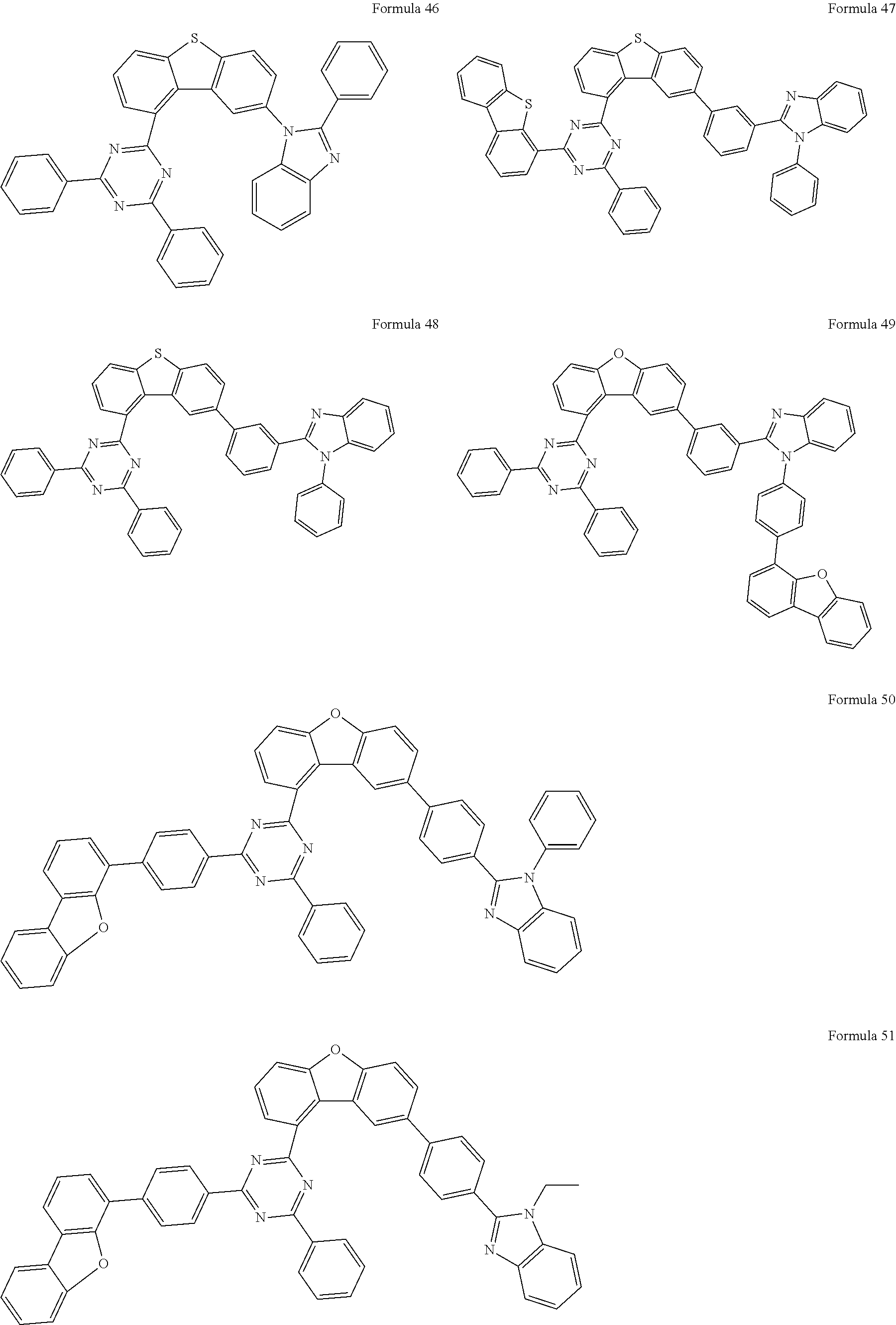

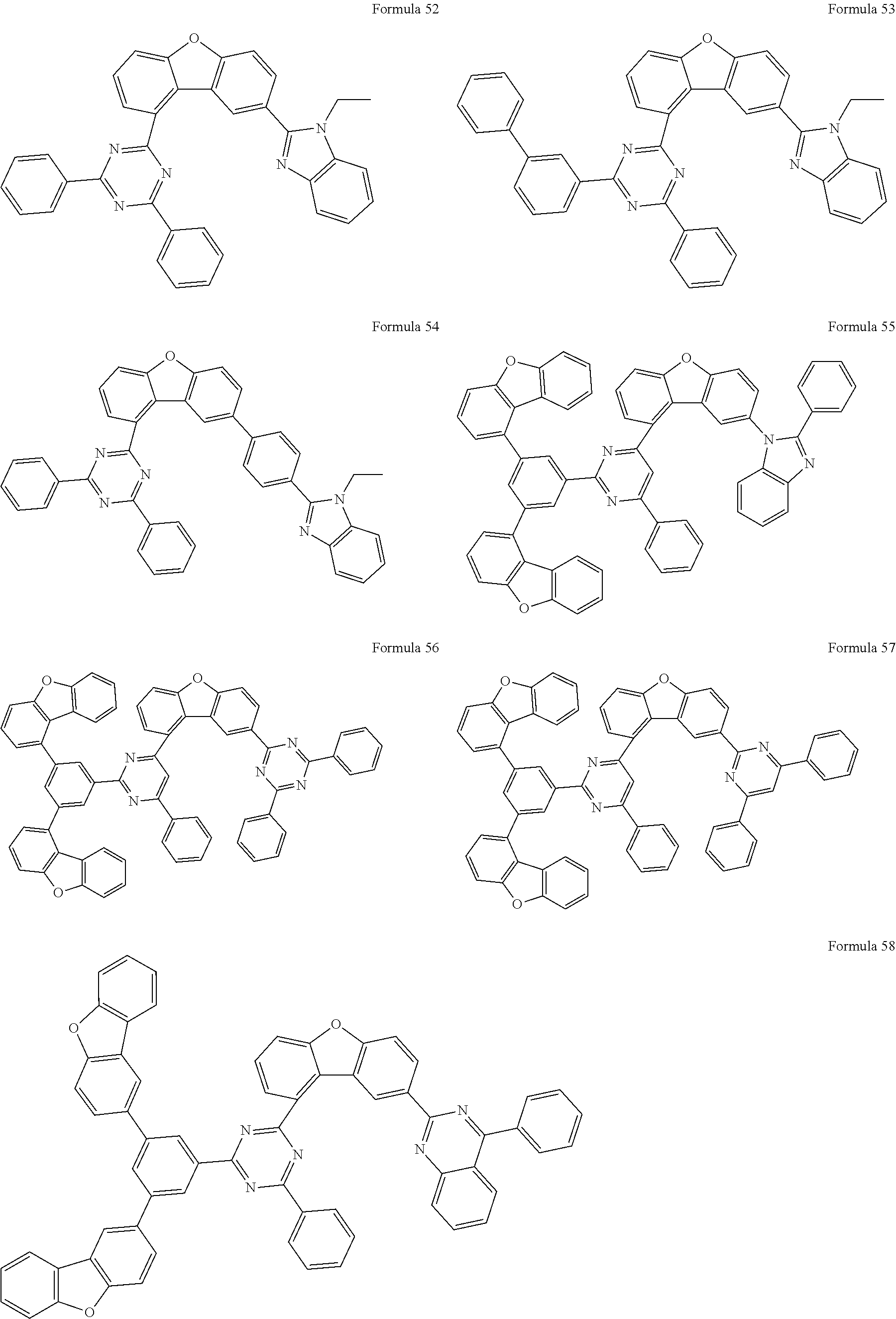

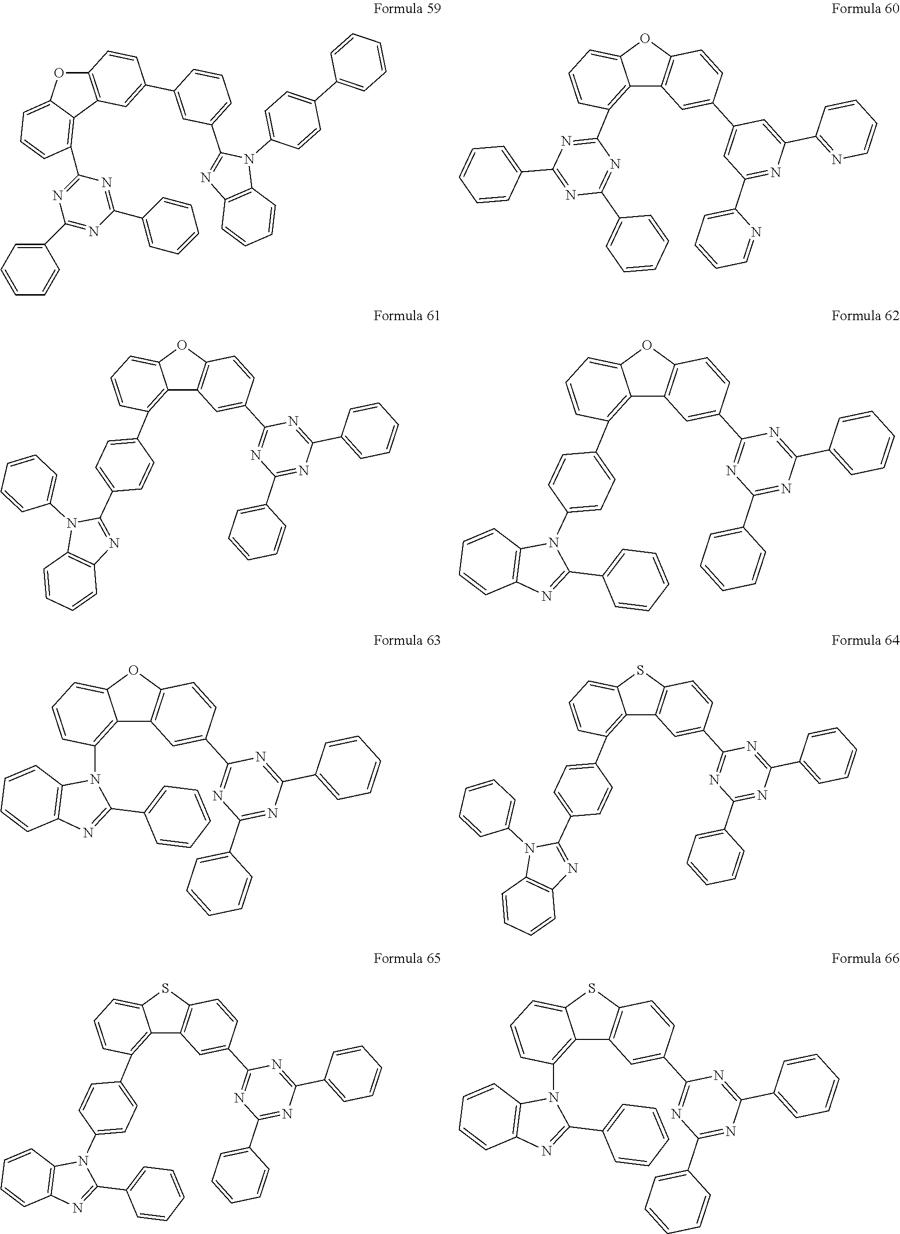

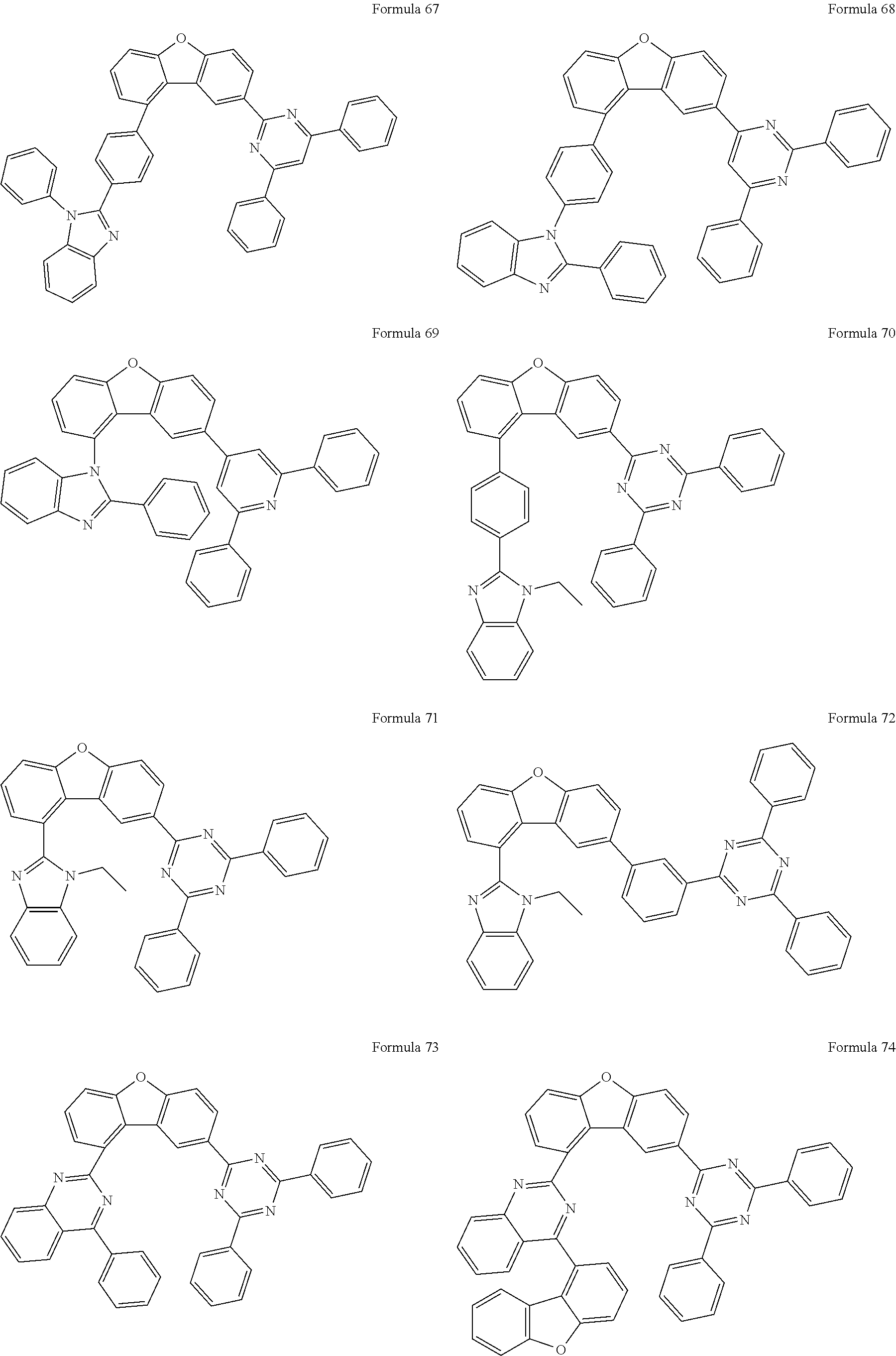

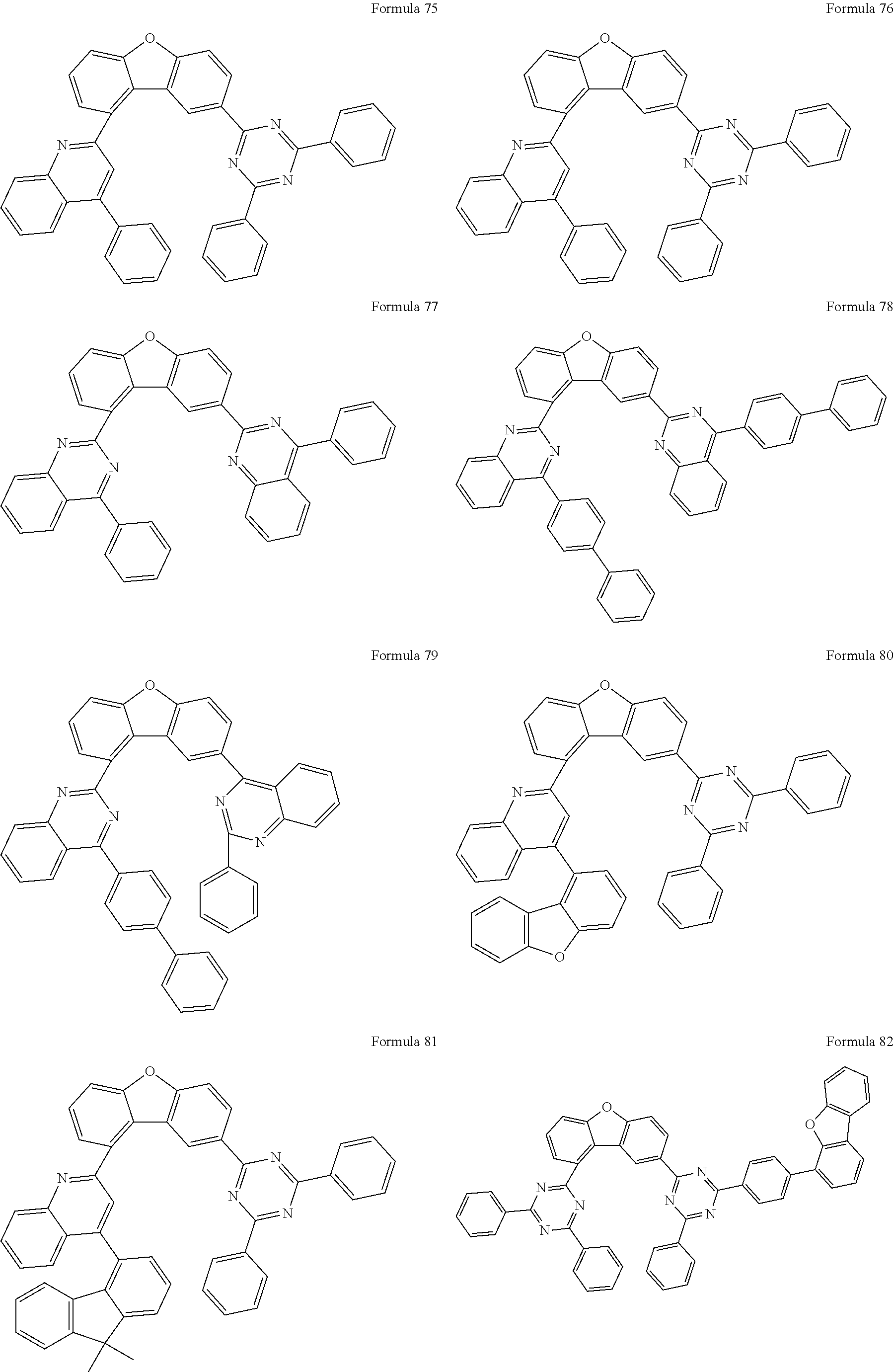

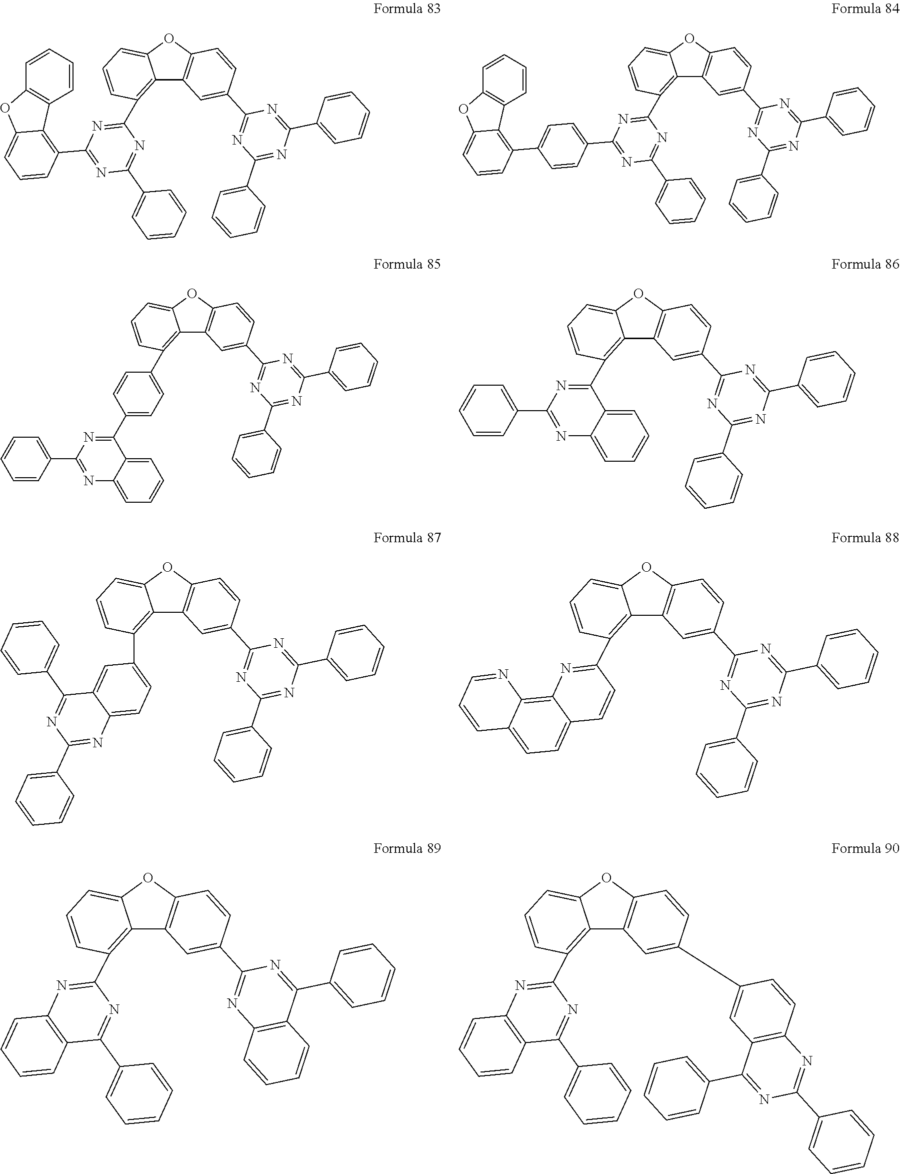

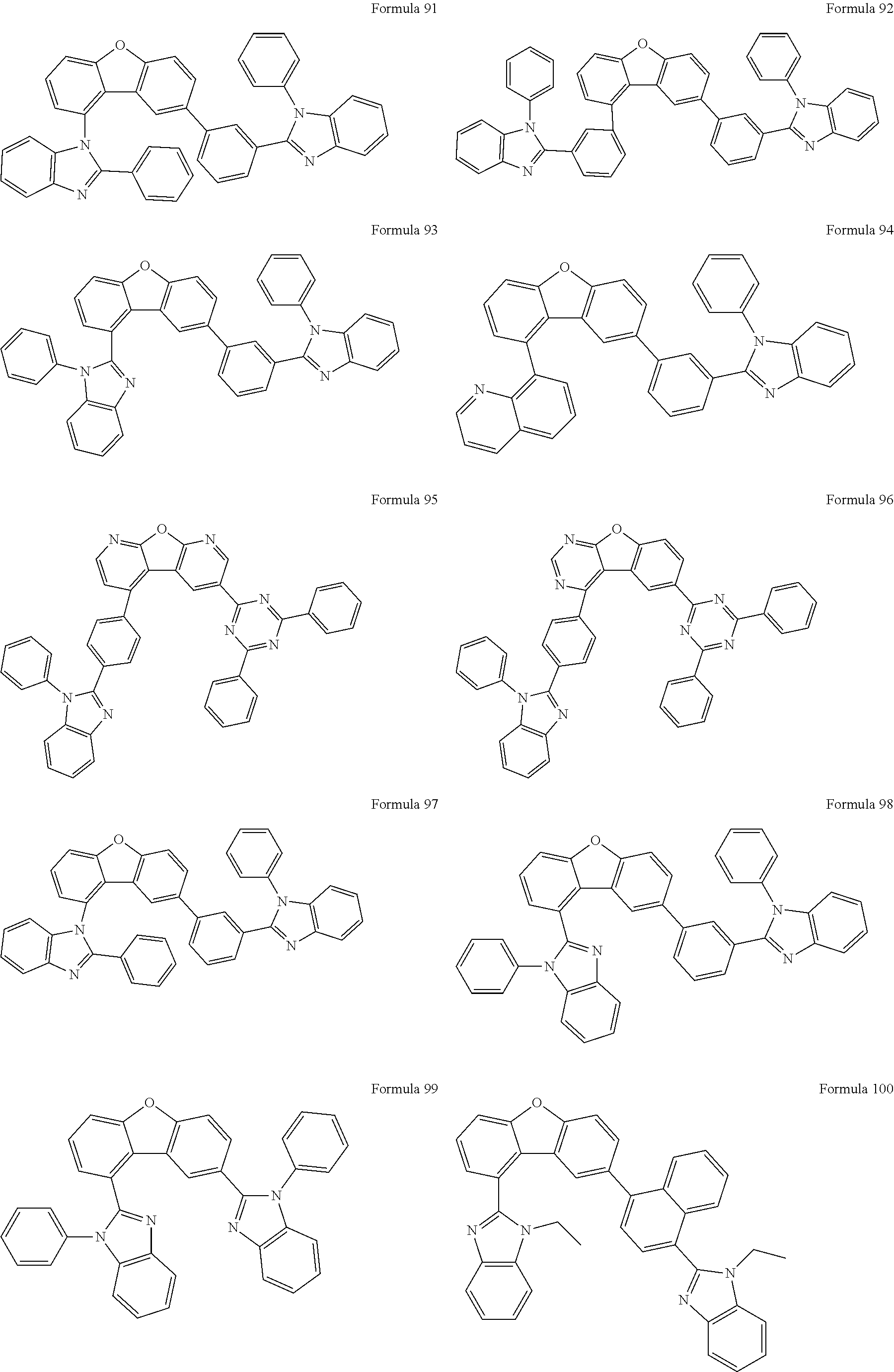

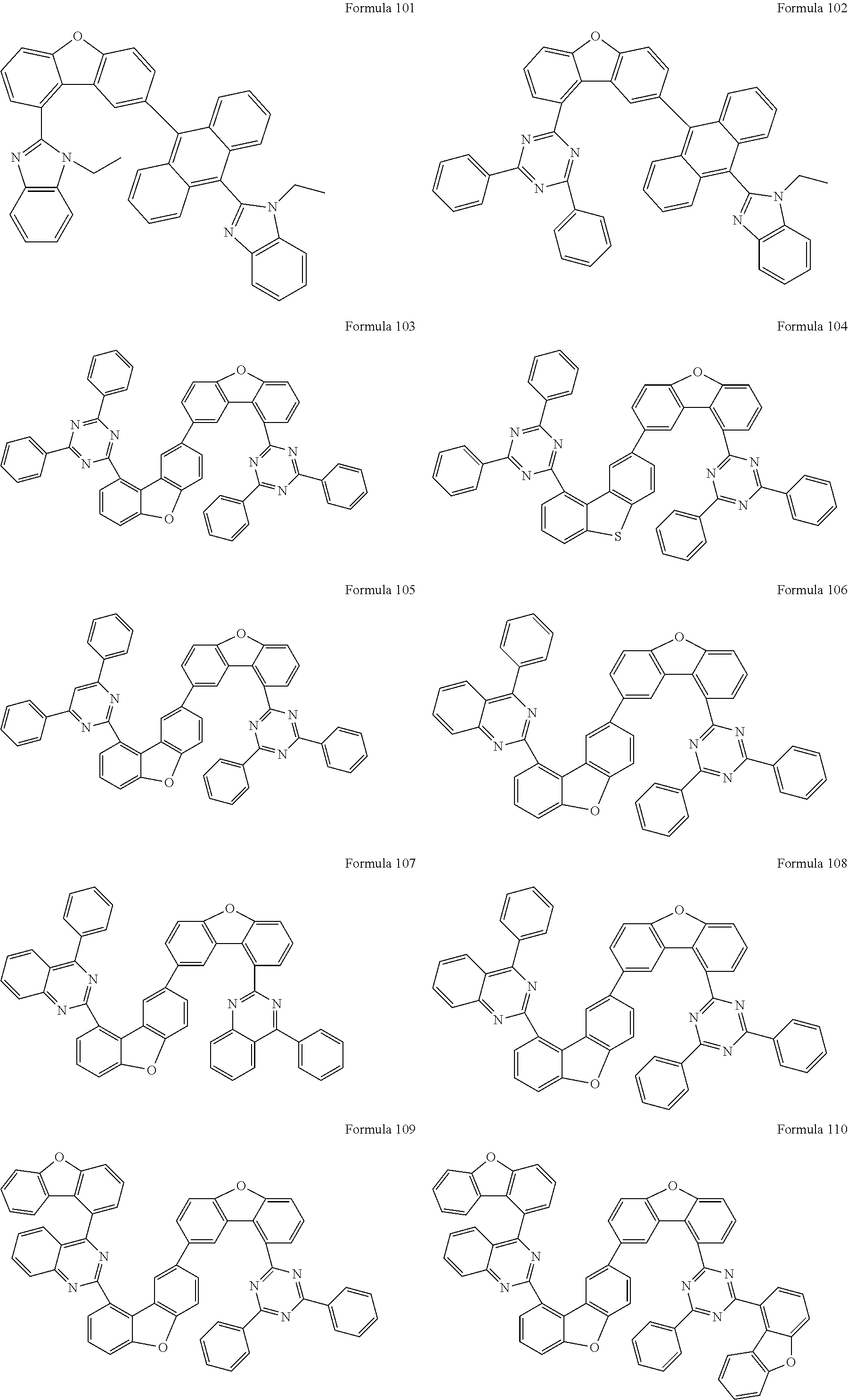

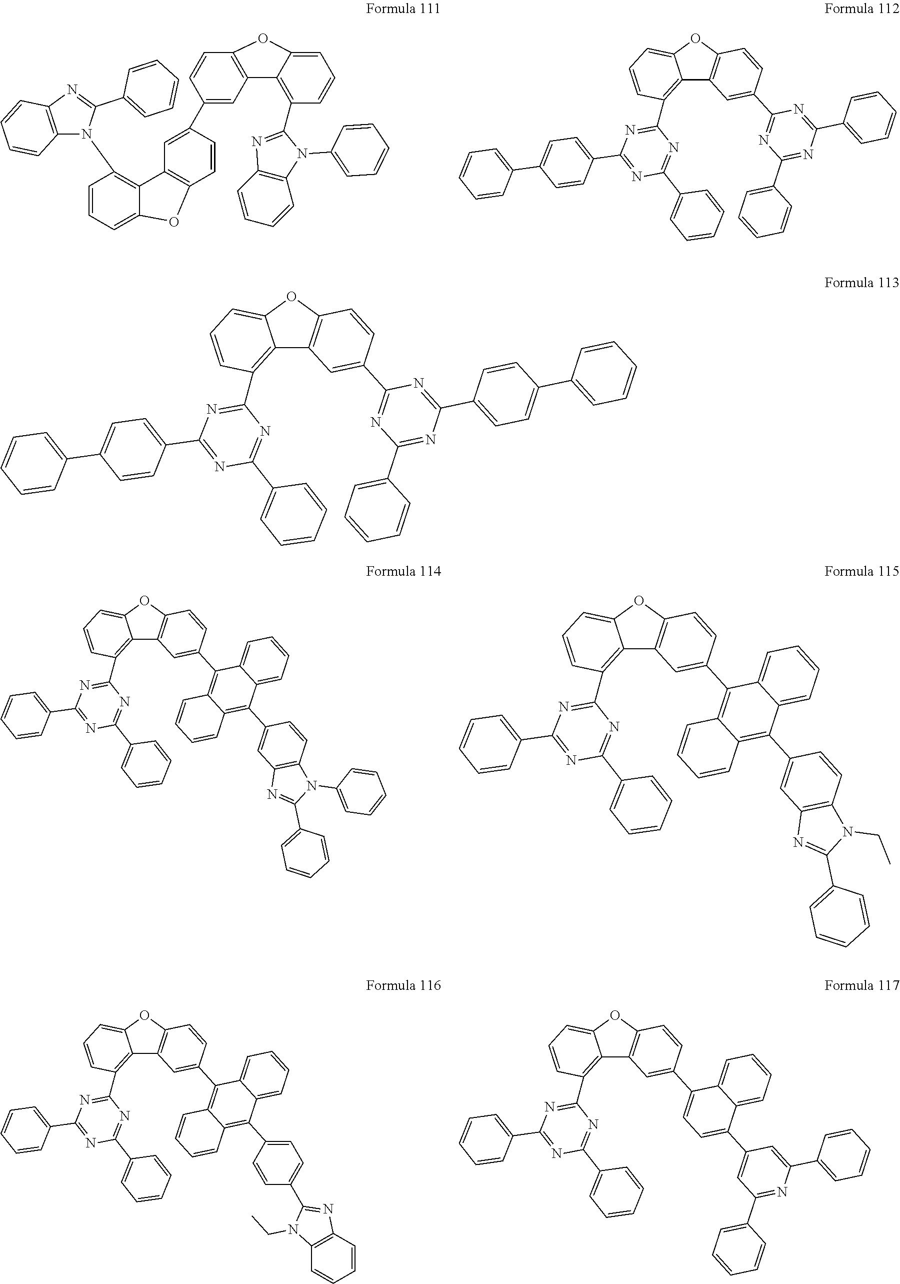

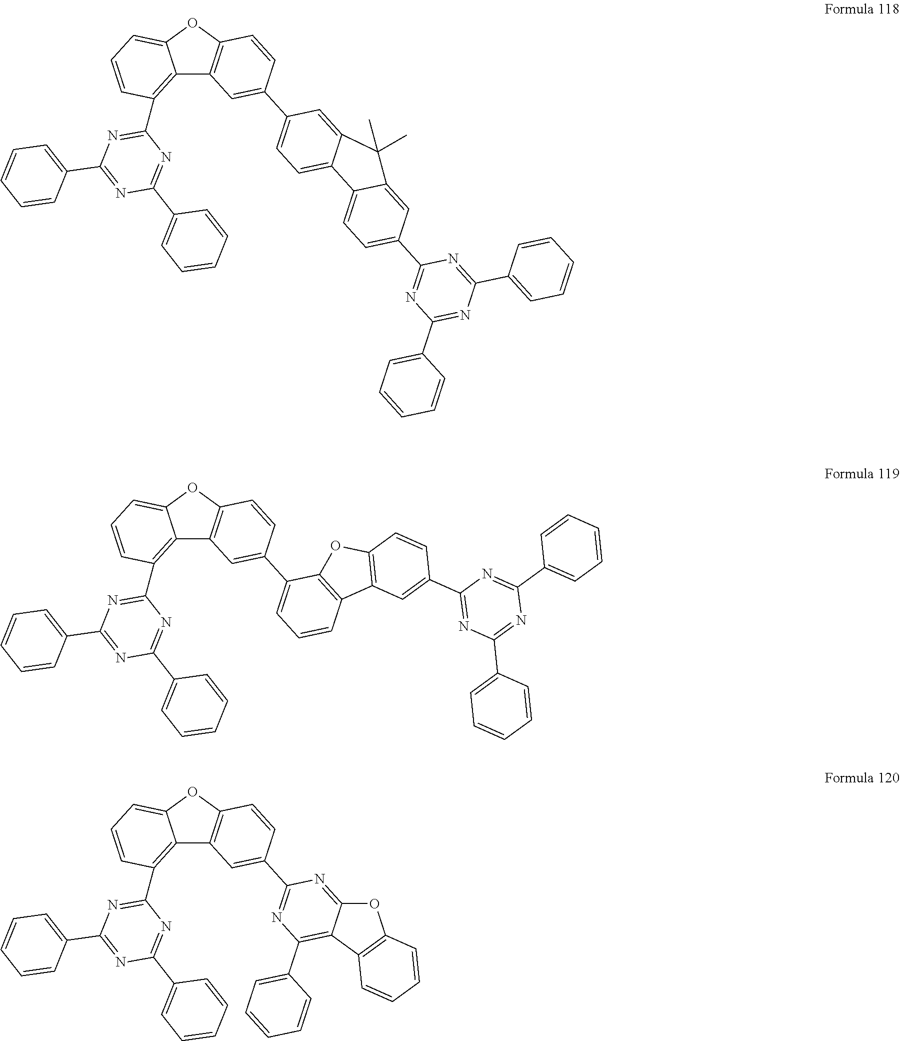 [SMILES: CC1(C)c2cc(-c3ccc4oc5cccc(-c6nc(-c7ccccc7)nc(-c7ccccc7)n6)c5c4c3)ccc2-c2ccc(-c3nc(-c4ccccc4)nc(-c4ccccc4)n3)cc21 ; c1ccc(-c2nc(-c3ccccc3)nc(-c3ccc4oc5c(-c6ccc7oc8cccc(-c9nc(-c%10ccccc%10)nc(-c%10ccccc%10)n9)c8c7c6)cccc5c4c3)n2)cc1 ; c1ccc(-c2nc(-c3ccccc3)nc(-c3cccc4oc5ccc(-c6nc(-c7ccccc7)c7c(n6)oc6ccccc67)cc5c34)n2)cc1]

Preferred embodiments of compounds of the invention are detailed specifically in the examples, these compounds being usable alone or in combination with further compounds for all purposes of the invention.

Provided that the conditions specified in claim 1 are complied with, the abovementioned preferred embodiments can be combined with one another as desired. In a particularly preferred embodiment of the invention, the abovementioned preferred embodiments apply simultaneously.

The compounds of the invention are preparable in principle by various processes. However, the processes described hereinafter have been found to be particularly suitable.

Therefore, the present invention further provides a process for preparing the compounds comprising structures of formula (I) in which, in a coupling reaction, a compound comprising at least one electron-transporting group is joined to a compound comprising at least one benzofuran and/or benzothiophene radical.

Suitable compounds having an electron-transporting group are in many cases commercially available, and the starting compounds detailed in the examples are obtainable by known processes, and so reference is made thereto.

These compounds can be reacted with further aryl compounds by known coupling reactions, the necessary conditions for this purpose being known to the person skilled in the art, and detailed specifications in the examples give support to the person skilled in the art in conducting these reactions.

Particularly suitable and preferred coupling reactions which all lead to C—C bond formation and/or C—N bond formation are those according to BUCHWALD, SUZUKI, YAMAMOTO, STILLE, HECK, NEGISHI, SONOGASHIRA and HIYAMA. These reactions are widely known, and the examples will provide the person skilled in the art with further pointers.

In all the synthesis schemes which follow, the compounds are shown with a small number of substituents to simplify the structures. This does not rule out the presence of any desired further substituents in the processes.

An illustrative implementation is given by the schemes which follow, without any intention that these should impose a restriction. The component steps of the individual schemes may be combined with one another as desired.

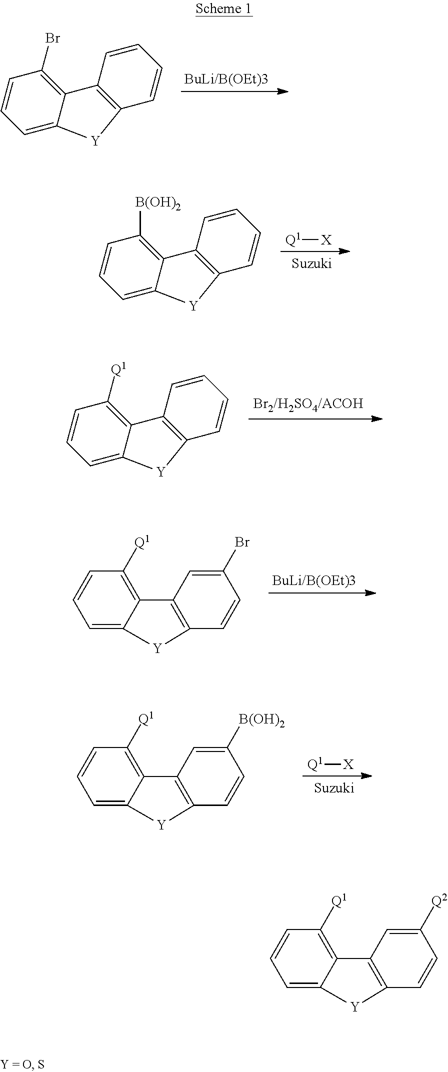

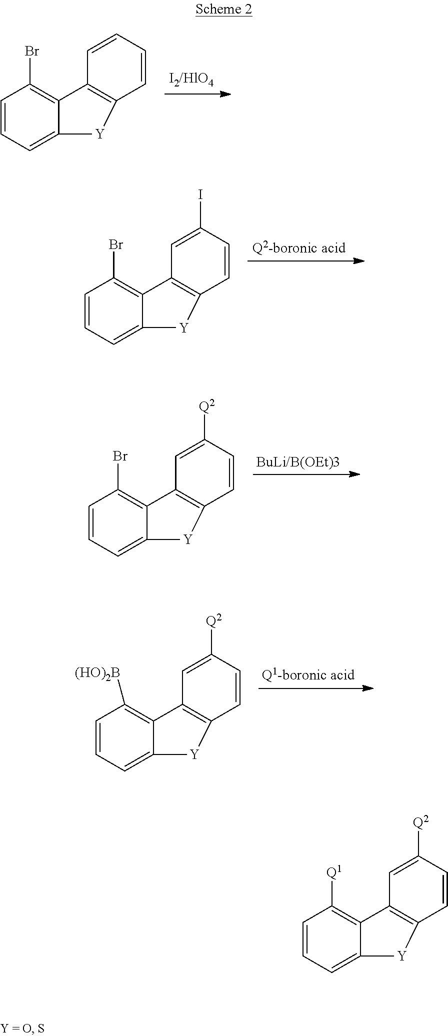

In Schemes 1 and 2, the radical detailed under the definition of Q¹ and/or Q² is an electron-conducting group as defined above.

The processes shown for synthesis of the compounds of the invention should be understood by way of example. The person skilled in the art will be able to develop alternative synthesis routes within the scope of his common knowledge in the art.

The principles of the preparation processes detailed above are known in principle from the literature for similar compounds and can be adapted easily by the person skilled in the art to the preparation of the compounds of the invention. Further information can be found in the examples.

It is possible by these processes, if necessary followed by purification, for example recrystallization or sublimation, to obtain the compounds of the invention comprising structures of formula (I) in high purity, preferably more than 99% (determined by means of ¹H NMR and/or HPLC).

The compounds of the invention may also have suitable substituents, for example relatively long alkyl groups (about 4 to 20 carbon atoms), especially branched alkyl groups, or optionally substituted aryl groups, for example xylyl, mesityl or branched terphenyl or quaterphenyl groups, which bring about solubility in standard organic solvents, for example toluene or xylene, at room temperature in a sufficient concentration soluble, in order to be able to process the compounds from solution. These soluble compounds are of particularly good suitability for processing from solution, for example by printing methods. In addition, it should be emphasized that the compounds of the invention comprising at least one structure of the formula (I) already have enhanced solubility in these solvents.

The compounds of the invention may also be mixed with a polymer. It is likewise possible to incorporate these compounds covalently into a polymer. This is especially possible with compounds substituted by reactive leaving groups such as bromine, iodine, chlorine, boronic acid or boronic ester, or by reactive polymerizable groups such as olefins or oxetanes. These may find use as monomers for production of corresponding oligomers, dendrimers or polymers. The oligomerization or polymerization is preferably effected via the halogen functionality or the boronic acid functionality or via the polymerizable group. It is additionally possible to crosslink the polymers via groups of this kind. The compounds of the invention and polymers may be used in the form of a crosslinked or uncrosslinked layer.

The invention therefore further provides oligomers, polymers or dendrimers containing one or more of the above-detailed structures of the formula (I) or compounds of the invention, wherein one or more bonds of the compounds of the invention or of the structures of the formula (I) to the polymer, oligomer or dendrimer are present. According to the linkage of the structures of the formula (I) or of the compounds, these therefore form a side chain of the oligomer or polymer or are bonded within the main chain. The polymers, oligomers or dendrimers may be conjugated, partly conjugated or nonconjugated. The oligomers or polymers may be linear, branched or dendritic. For the repeat units of the compounds of the invention in oligomers, dendrimers and polymers, the same preferences apply as described above.

For preparation of the oligomers or polymers, the monomers of the invention are homopolymerized or copolymerized with further monomers. Preference is given to copolymers wherein the units of formula (I) or the preferred embodiments recited above and hereinafter are present to an extent of 0.01 to 99.9 mol %, preferably 5 to 90 mol %, more preferably 20 to 80 mol %. Suitable and preferred comonomers which form the polymer base skeleton are chosen from fluorenes (for example according to EP 842208 or WO 2000/022026), spirobifluorenes (for example according to EP 707020, EP 894107 or WO 2006/061181), paraphenylenes (for example according to WO 92/18552), carbazoles (for example according to WO 2004/070772 or WO 2004/113468), thiophenes (for example according to EP 1028136), dihydrophenanthrenes (for example according to WO 2005/014689), cis- and trans-indenofluorenes (for example according to WO 2004/041901 or WO 2004/113412), ketones (for example according to WO 2005/040302), phenanthrenes (for example according to WO 2005/104264 or WO 2007/017066) or else a plurality of these units. The polymers, oligomers and dendrimers may contain still further units, for example hole transport units, especially those based on triarylamines, and/or electron transport units.

Additionally of particular interest are compounds of the invention which feature a high glass transition temperature. In this connection, preference is given especially to compounds of the invention comprising structures of the general formula (I) or the preferred embodiments recited above and hereinafter which have a glass transition temperature of at least 70° C., more preferably of at least 110° C., even more preferably of at least 125° C. and especially preferably of at least 150° C., determined in accordance with DIN 51005 (2005-08 version).

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, hexamethylindane or mixtures of these solvents.

The present invention therefore further provides a formulation comprising a compound of the invention and at least one further compound. The further compound may, for example, be a solvent, especially one of the abovementioned solvents or a mixture of these solvents. The further compound may alternatively be at least one further organic or inorganic compound which is likewise used in the electronic device, for example an emitting compound, especially a phosphorescent dopant, and/or a further matrix material. This further compound may also be polymeric.

The present invention therefore still further provides a composition comprising a compound of the invention and at least one further organically functional material. Functional materials are generally the organic or inorganic materials introduced between the anode and cathode. Preferably, the organic functional material is selected from the group consisting of fluorescent emitters, phosphorescent emitters, host materials, matrix materials, electron transport materials, electron injection materials, hole conductor materials, hole injection materials, n-dopants, wide band gap materials, electron blocker materials and hole blocker materials.

The present invention therefore also relates to a composition comprising at least one compound comprising structures of formula (I) or the preferred embodiments recited above and hereinafter and at least one further matrix material. According to a particular aspect of the present invention, the further matrix material has hole-transporting properties.

The present invention further provides a composition comprising at least one compound comprising at least one structure of formula (I) or the preferred embodiments recited above and hereinafter and at least one wide band gap material, a wide band gap material being understood to mean a material in the sense of the disclosure of U.S. Pat. No. 7,294,849. These systems exhibit particular advantageous performance data in electroluminescent devices.

Preferably, the additional compound may have a band gap of 2.5 eV or more, preferably 3.0 eV or more, very preferably of 3.5 eV or more. One way of calculating the band gap is via the energy levels of the highest occupied molecular orbital (HOMO) and the lowest unoccupied molecular orbital (LUMO).

Molecular orbitals, especially also the highest occupied molecular orbital (HOMO) and the lowest unoccupied molecular orbital (LUMO), the energy levels thereof and the energy of the lowest triplet state T₁ and that of the lowest excited singlet state S₁ of the materials are determined via quantum-chemical calculations. For calculation of organic substances without metals, an optimization of geometry is first conducted by the “Ground State/Semi-empirical/Default Spin/AM1/Charge 0/Spin Singlet” method. Subsequently, an energy calculation is effected on the basis of the optimized geometry. This is done using the “TD-SCF/DFT/Default Spin/B3PW91” method with the “6-31G(d)” basis set (charge 0, spin singlet). For metal-containing compounds, the geometry is optimized via the “Ground State/Hartree-Fock/Default Spin/LanL2 MB/Charge 0/Spin Singlet” method. The energy calculation is effected analogously to the above-described method for the organic substances, except that the “LanL2DZ” basis set is used for the metal atom and the “6-31G(d)” basis set for the ligands. The HOMO energy level HEh or LUMO energy level LEh is obtained from the energy calculation in Hartree units. This is used to determine the HOMO and LUMO energy levels in electron volts, calibrated by cyclic voltammetry measurements, as follows:

HOMO(eV)=((HEh*27.212)−0.9899)/1.1206

LUMO(eV)=((LEh*27.212)−2.0041)/1.385

These values are to be regarded as HOMO and LUMO energy levels of the materials in the context of this application.

The lowest triplet state T₁ is defined as the energy of the triplet state having the lowest energy, which is apparent from the quantum-chemical calculation described.

The lowest excited singlet state S₁ is defined as the energy of the excited singlet state having the lowest energy, which is apparent from the quantum-chemical calculation described.

The method described herein is independent of the software package used and always gives the same results. Examples of frequently utilized programs for this purpose are “Gaussian09 W” (Gaussian Inc.) and Q-Chem 4.1 (Q-Chem, Inc.).

The present invention also relates to a composition comprising at least one compound comprising structures of formula (I) or the preferred embodiments recited above and hereinafter and at least one phosphorescent emitter, the term “phosphorescent emitter” also being understood to mean phosphorescent dopants.

A dopant in a system comprising a matrix material and a dopant is understood to mean that component having the smaller proportion in the mixture. Correspondingly, a matrix material in a system comprising a matrix material and a dopant is understood to mean that component having the greater proportion in the mixture.

Preferred phosphorescent dopants for use in matrix systems, preferably mixed matrix systems, are the preferred phosphorescent dopants specified hereinafter.

The term “phosphorescent dopants” typically encompasses compounds where the emission of light is effected through a spin-forbidden transition, for example a transition from an excited triplet state or a state having a higher spin quantum number, for example a quintet state.

Suitable phosphorescent compounds (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum. In the context of the present invention, all luminescent compounds containing the abovementioned metals are regarded as phosphorescent compounds.

Examples of the above-described emitters can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960 and the as yet unpublished applications EP 13004411.8, EP 14000345.0, EP 14000417.7 and EP 14002623.8. In general, all phosphorescent complexes as used for phosphorescent OLEDs according to the prior art and as known to those skilled in the art in the field of organic electroluminescence are suitable, and the person skilled in the art will be able to use further phosphorescent complexes without exercising inventive skill.

Explicit examples of phosphorescent dopants are adduced in the following table:

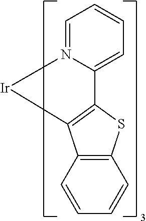

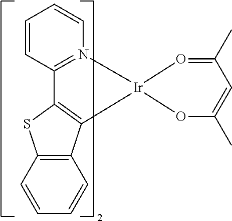

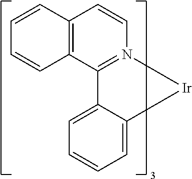

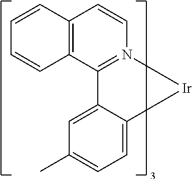

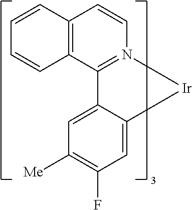

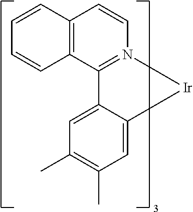

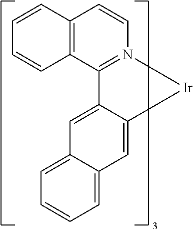

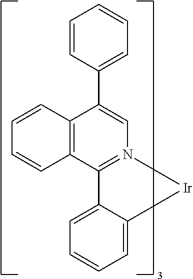

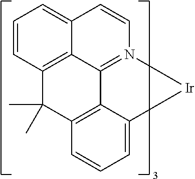

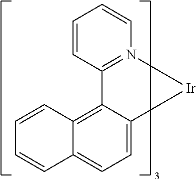

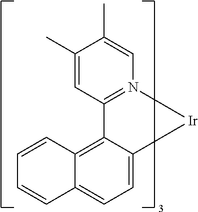

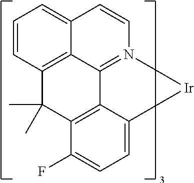

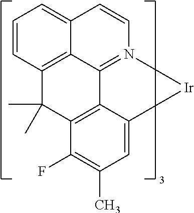

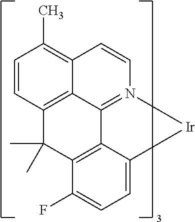

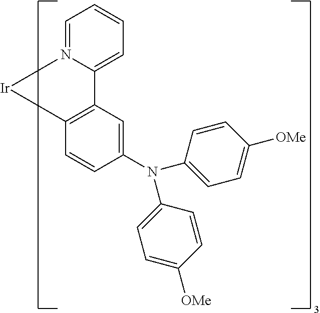

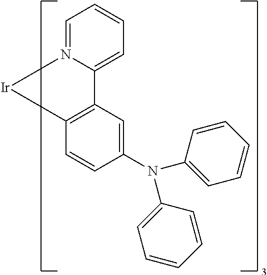

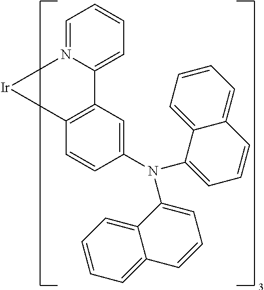

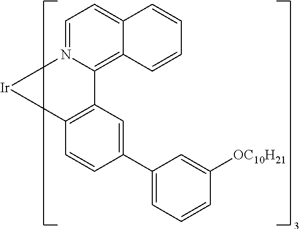

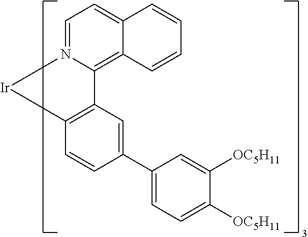

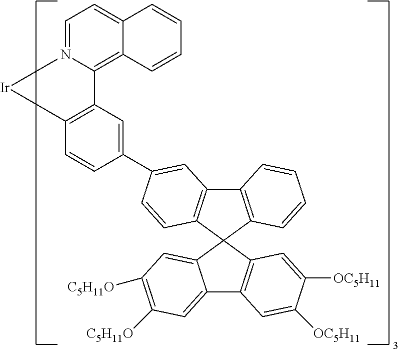

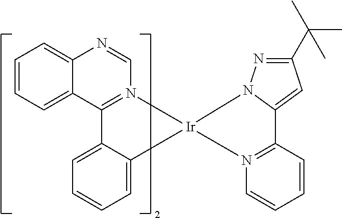

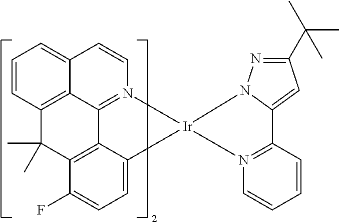

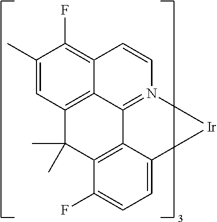

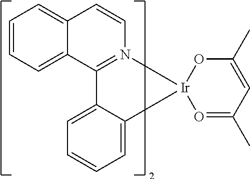

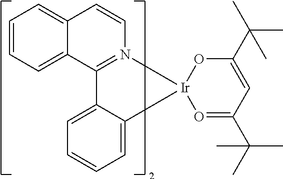

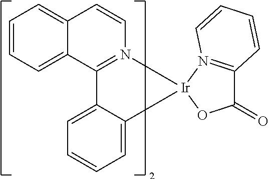

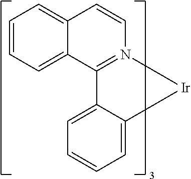

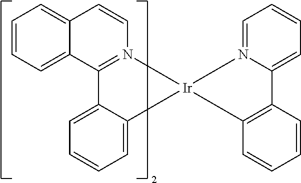

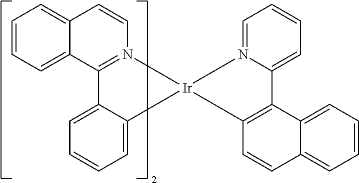

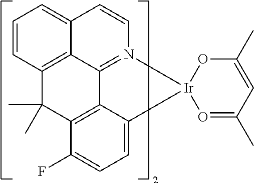

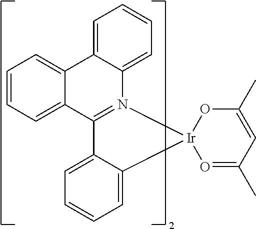

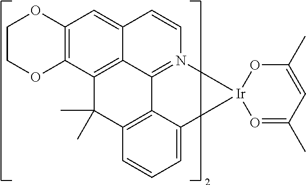

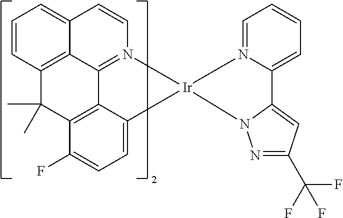

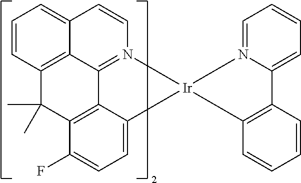

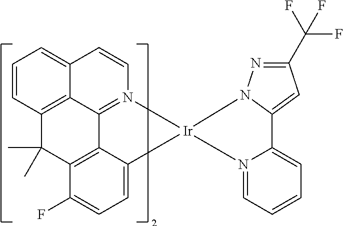

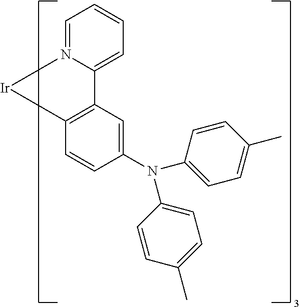

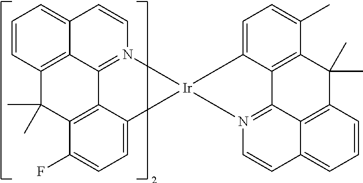

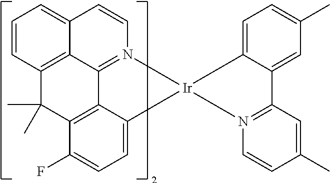

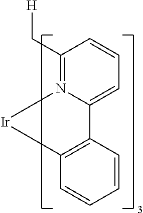

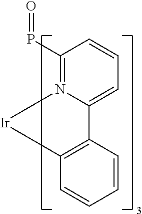

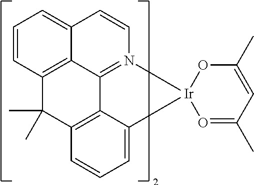

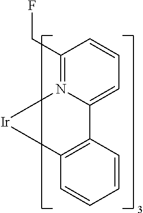

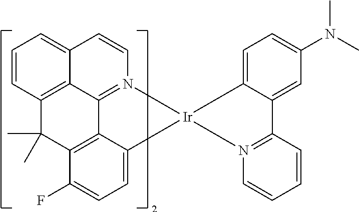

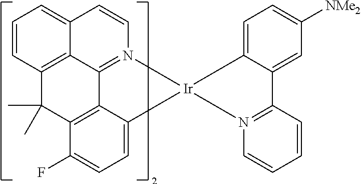

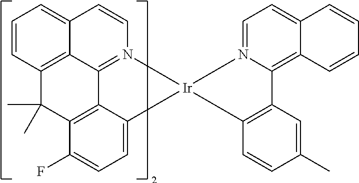

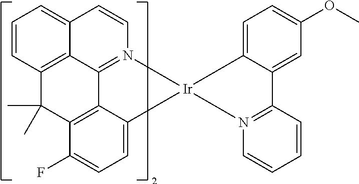

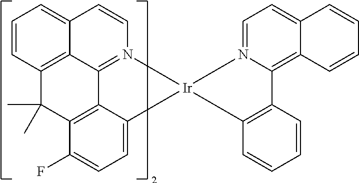

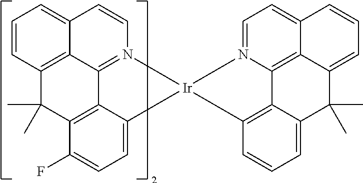

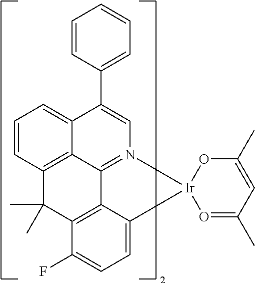

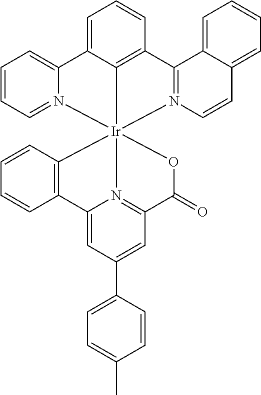

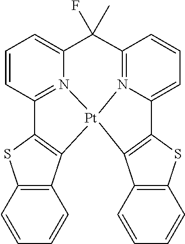

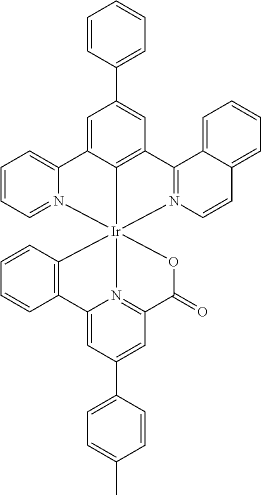

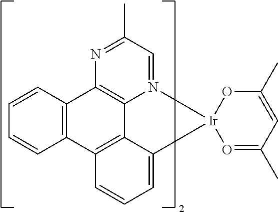

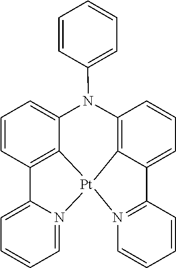

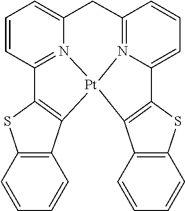

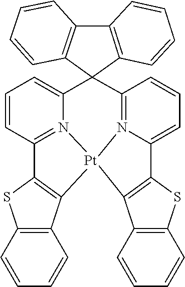

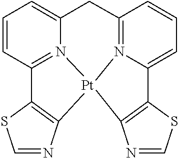

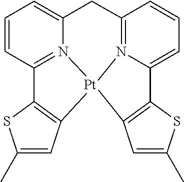

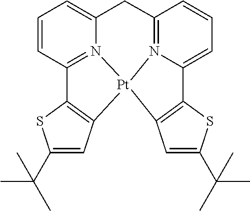

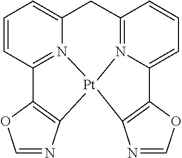

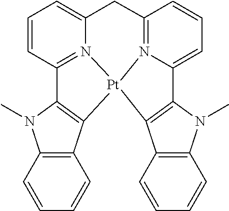

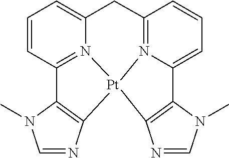

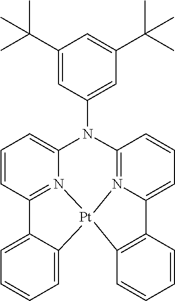

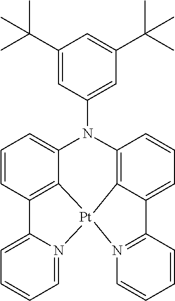

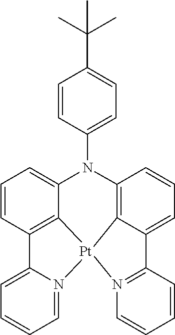

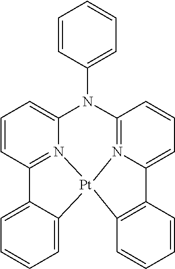

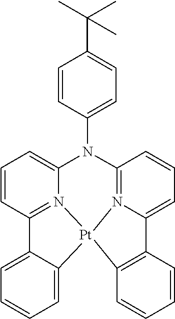

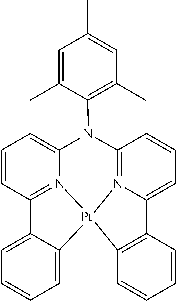

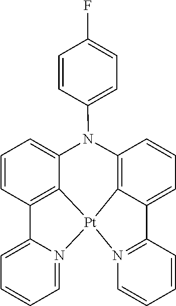

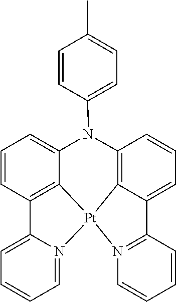

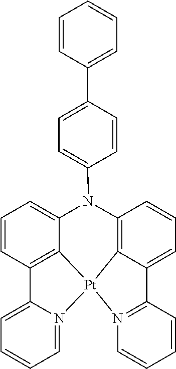

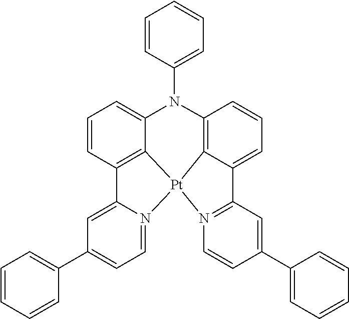

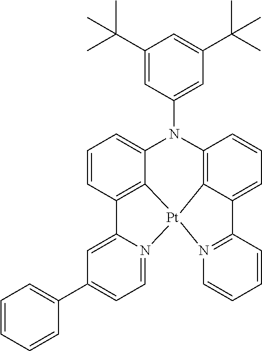

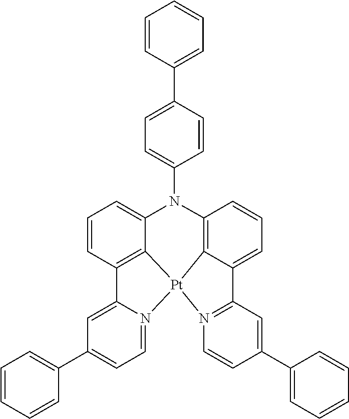

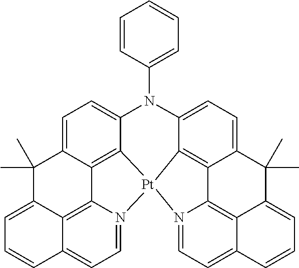

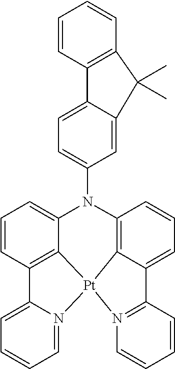

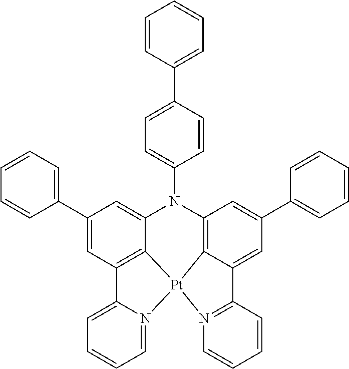

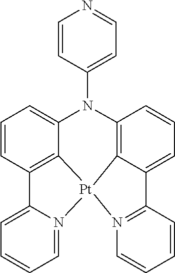

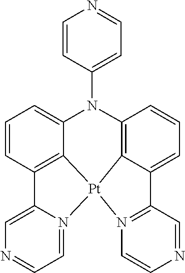

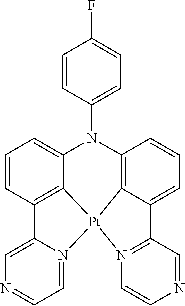

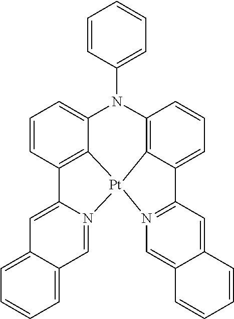

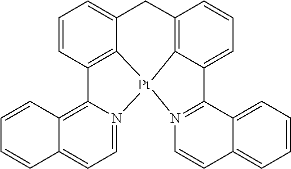

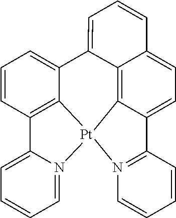

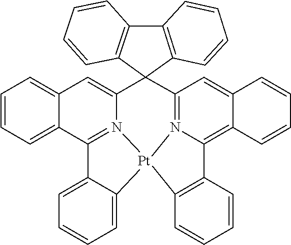

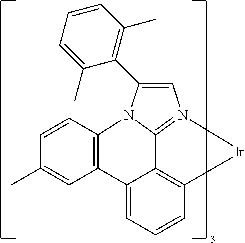

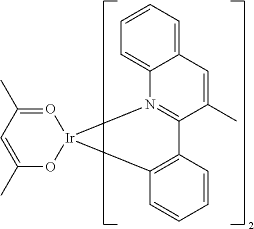

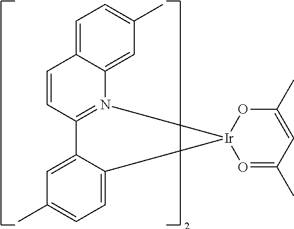

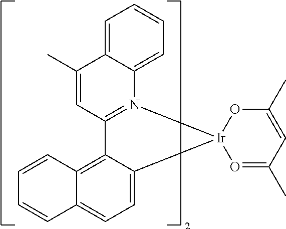

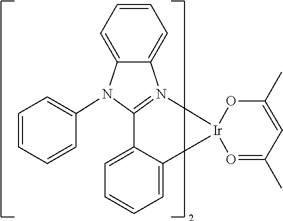

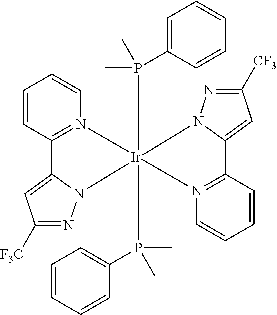

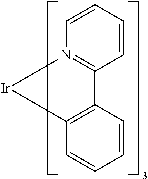

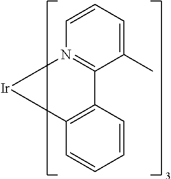

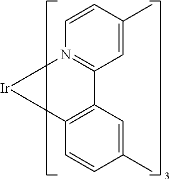

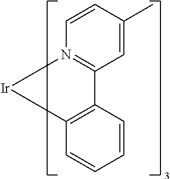

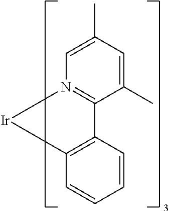

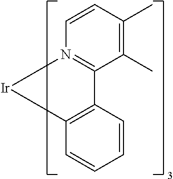

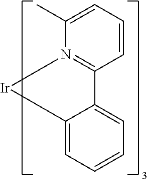

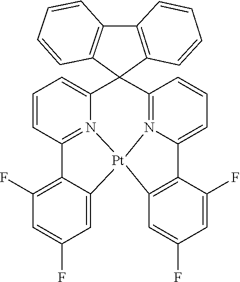

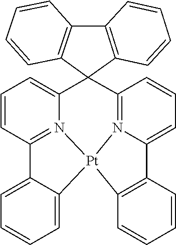

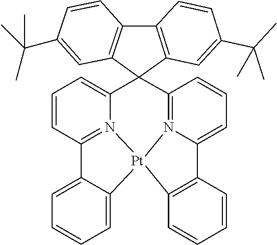

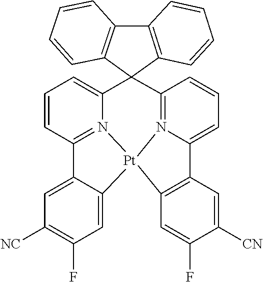

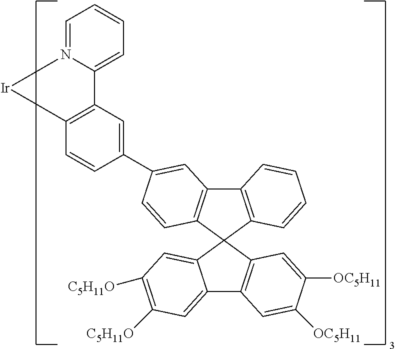

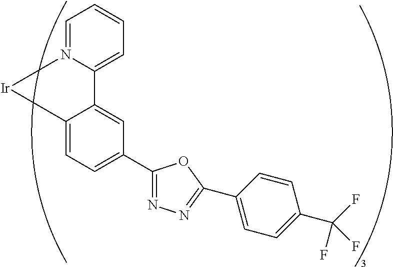

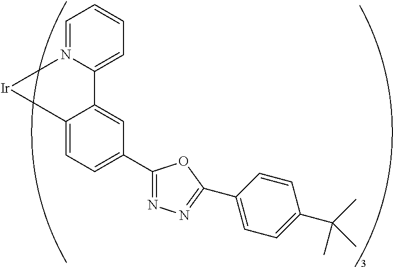

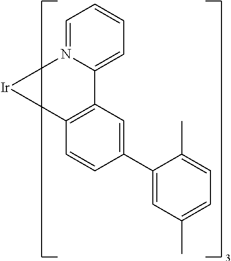

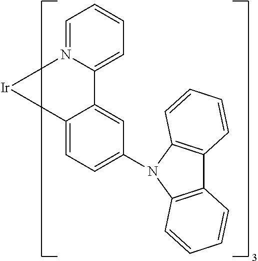

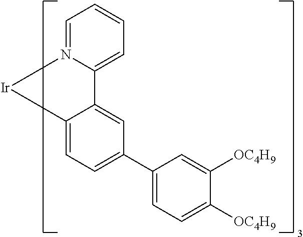

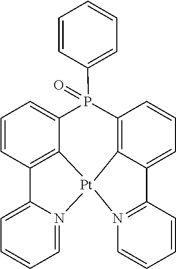

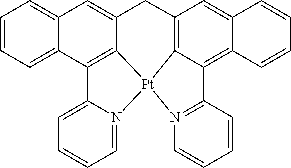

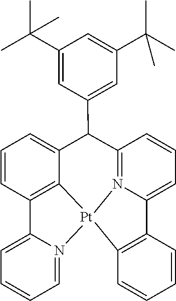

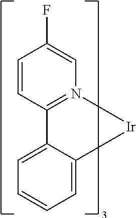

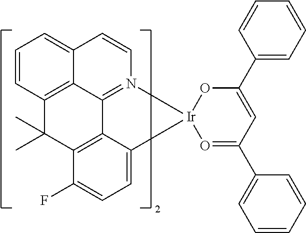

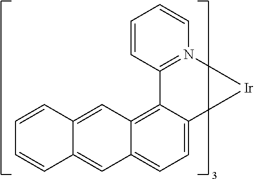

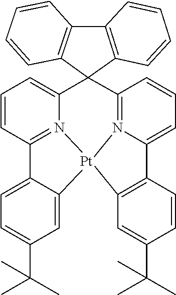

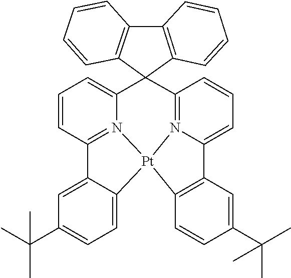

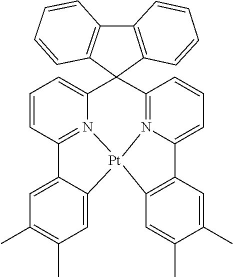

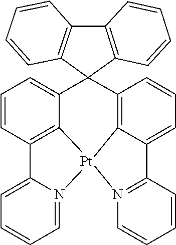

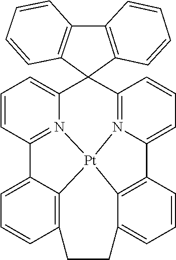

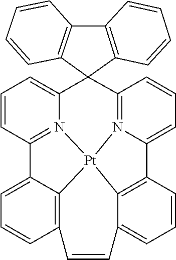

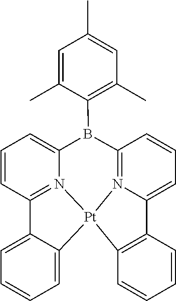

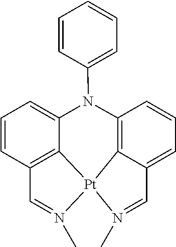

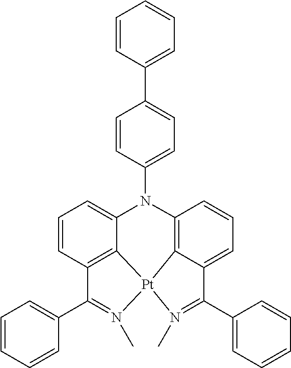

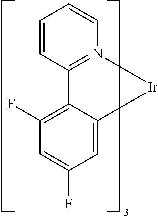

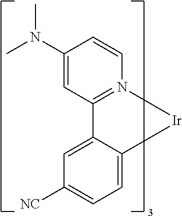

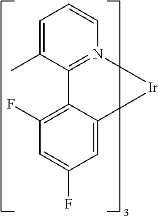

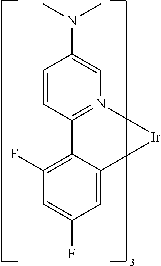

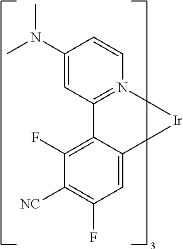

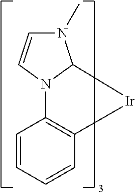

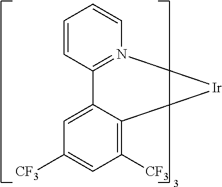

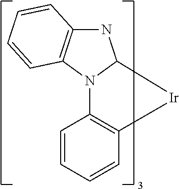

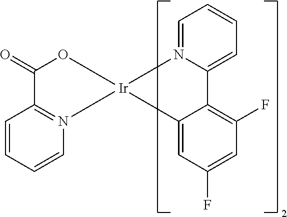

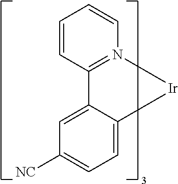

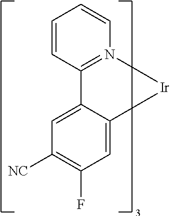

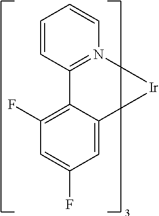

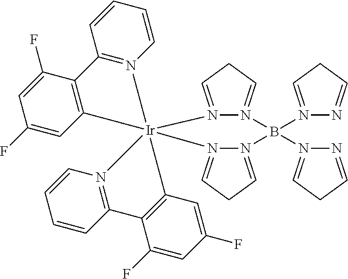

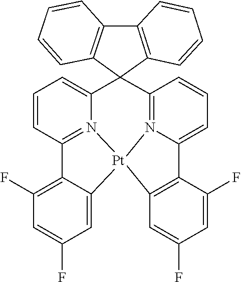

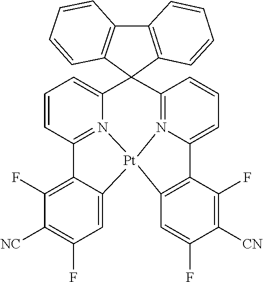

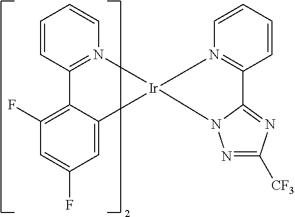

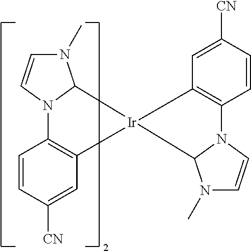

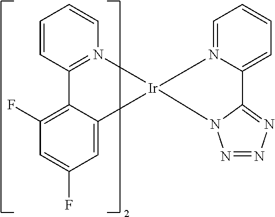

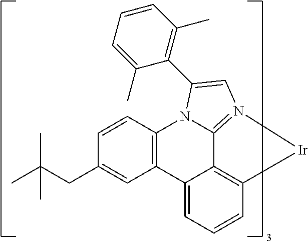

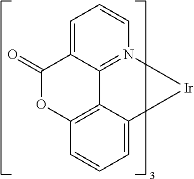

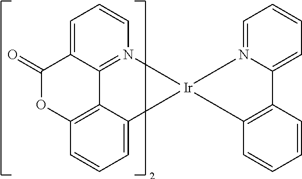

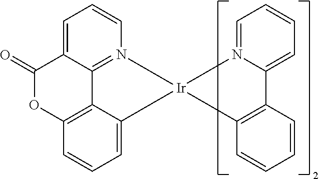

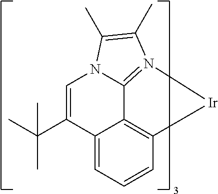

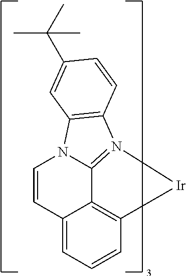

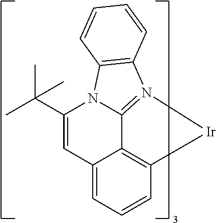

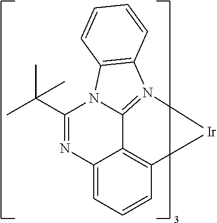

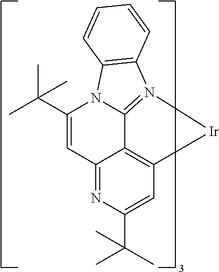

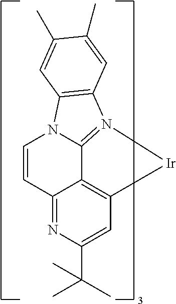

The above-described compound comprising structures of the formula (I) or the above-detailed preferred embodiments can preferably be used as active component in an electronic device. An electronic device is understood to mean any device comprising anode, cathode and at least one layer between anode and cathode, said layer comprising at least one organic or organometallic compound. The electronic device of the invention thus comprises anode, cathode and at least one intervening layer containing at least one compound comprising structures of the formula (I).

Preferred electronic devices here are selected from the group consisting of organic electroluminescent devices (OLEDs, PLEDs), organic integrated circuits (O-ICs), organic field-effect transistors (O-FETs), organic thin-film transistors (O-TFTs), organic light-emitting transistors (O-LETs), organic solar cells (O-SCs), organic optical detectors, organic photoreceptors, organic field-quench devices (O-FQDs), organic electrical sensors, light-emitting electrochemical cells (LECs), organic laser diodes (O-lasers) and organic plasmon emitting devices (D. M. Koller et al., Nature Photonics 2008, 1-4), preferably organic electroluminescent devices (OLEDs, PLEDs), especially phosphorescent OLEDs, containing at least one compound comprising structures of the formula (I) in at least one layer. Particular preference is given to organic electroluminescent devices. Active components are generally the organic or inorganic materials introduced between the anode and cathode, for example charge injection, charge transport or charge blocker materials, but especially emission materials and matrix materials.

A preferred embodiment of the invention is organic electroluminescent devices. The organic electroluminescent device comprises cathode, anode and at least one emitting layer. Apart from these layers, it may comprise still further layers, for example in each case one or more hole injection layers, hole transport layers, hole blocker layers, electron transport layers, electron injection layers, exciton blocker layers, electron blocker layers, charge generation layers and/or organic or inorganic p/n junctions. At the same time, it is possible that one or more hole transport layers are p-doped, for example with metal oxides such as MoO₃ or WO₃ or with (per)fluorinated electron-deficient aromatic systems, and/or that one or more electron transport layers are n-doped. It is likewise possible for interlayers to be introduced between two emitting layers, these having, for example, an exciton-blocking function and/or controlling the charge balance in the electroluminescent device. However, it should be pointed out that not necessarily every one of these layers need be present.

In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are three-layer systems where the three layers exhibit blue, green and orange or red emission (for the basic construction see, for example, WO 2005/011013), or systems having more than three emitting layers. The system may also be a hybrid system wherein one or more layers fluoresce and one or more other layers phosphoresce.

In a preferred embodiment of the invention, the organic electroluminescent device contains the compound of the invention comprising structures of formula (I) or the above-detailed preferred embodiments as matrix material, preferably as electron-conducting matrix material, in one or more emitting layers, preferably in combination with a further matrix material, preferably a hole-conducting matrix material. In a further preferred embodiment of the invention, the further matrix material is an electron-transporting compound.

In yet a further preferred embodiment, the further matrix material is a compound having a large band gap which is not involved to a significant degree, if at all, in the hole and electron transport in the layer. An emitting layer comprises at least one emitting compound.

Suitable matrix materials which can be used in combination with the compounds of formula (I) or according to the preferred embodiments are aromatic ketones, aromatic phosphine oxides or aromatic sulphoxides or sulphones, for example according to WO 2004/013080, WO 2004/093207, WO 2006/005627 or WO 2010/006680, triarylamines, especially monoamines, for example according to WO 2014/015935, carbazole derivatives, e.g. CBP (N,N-biscarbazolylbiphenyl) or the carbazole derivatives disclosed in WO 2005/039246, US 2005/0069729, JP 2004/288381, EP 1205527 or WO 2008/086851, indolocarbazole derivatives, for example according to WO 2007/063754 or WO 2008/056746, indenocarbazole derivatives, for example according to WO 2010/136109 and WO 2011/000455, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2010/015306, WO 2007/063754 or WO 2008/056746, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to US 2009/0136779, WO 2010/050778, WO 2011/042107, WO 2011/088877 or WO 2012/143080, triphenylene derivatives, for example according to WO 2012/048781, lactams, for example according to WO 2011/116865, WO 2011/137951 or WO 2013/064206, or 4-spirocarbazole derivatives, for example according to WO 2014/094963 or the as yet unpublished application EP 14002104.9. It is likewise possible for a further phosphorescent emitter which emits at a shorter wavelength than the actual emitter to be present as co-host in the mixture.

Preferred co-host materials are triarylamine derivatives, especially monoamines, indenocarbazole derivatives, 4-spirocarbazole derivatives, lactams and carbazole derivatives.

Preferred triarylamine derivatives which are used as co-host materials together with the compounds of the invention are selected from the compounds of the following formula (TA-1):

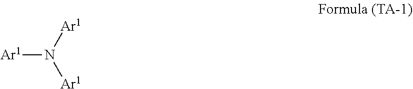

where Ar¹ is the same or different at each instance and has the definitions given above. Preferably, the Ar¹ groups are the same or different at each instance and are selected from the abovementioned R¹-1 to R¹-79 groups, more preferably R¹-1 to R¹-51.

In a preferred embodiment of the compounds of the formula (TA-1), at least one Ar¹ group is selected from a biphenyl group, which may be an ortho-, meta- or para-biphenyl group. In a further preferred embodiment of the compounds of the formula (TA-1), at least one Ar group is selected from a fluorene group or spirobifluorene group, where these groups may each be bonded to the nitrogen atom in the 1, 2, 3 or 4 position. In yet a further preferred embodiment of the compounds of the formula (TA-1), at least one Ar¹ group is selected from a phenylene or biphenyl group, where the group is an ortho-, meta- or para-bonded group, substituted by a dibenzofuran group, a dibenzothiophene group or a carbazole group, especially a dibenzofuran group, where the dibenzofuran or dibenzothiophene group is bonded to the phenylene or biphenyl group via the 1, 2, 3 or 4 position and where the carbazole group is bonded to the phenylene or biphenyl group via the 1, 2, 3 or 4 position or via the nitrogen atom.

In a particularly preferred embodiment of the compounds of the formula (TA-1), one Ar¹ group is selected from a fluorene or spirobifluorene group, especially a 4-fluorene or 4-spirobifluorene group, and one Ar¹ group is selected from a biphenyl group, especially a para-biphenyl group, or a fluorene group, especially a 2-fluorene group, and the third Ar¹ group is selected from a para-phenylene group or a para-biphenyl group, substituted by a dibenzofuran group, especially a 4-dibenzofuran group, or a carbazole group, especially an N-carbazole group or a 3-carbazole group.

Preferred indenocarbazole derivatives which are used as co-host materials together with the compounds of the invention are selected from the compounds of the following formula (TA-2):

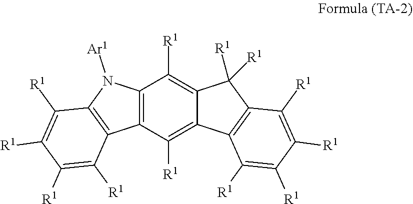

where Ar¹ and R¹ have the definitions listed above. Preferred embodiments of the Ar¹ group are the above-listed structures R¹-1 to R¹-79, more preferably R¹-1 to R¹-51.

A preferred embodiment of the compounds of the formula (TA-2) is the compounds of the following formula (TA-2a):

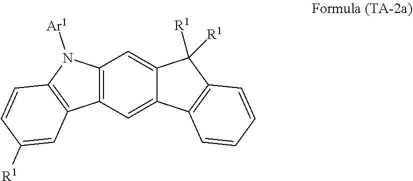

where Ar¹ and R¹ have the definitions given above. The two R¹ groups bonded to the indeno carbon atom here are preferably the same or different and are each an alkyl group having 1 to 4 carbon atoms, especially methyl groups, or an aromatic ring system having 6 to 12 carbon atoms, especially phenyl groups. More preferably, the two R¹ groups bonded to the indeno carbon atom are methyl groups. Further preferably, the R¹ substituent bonded to the indenocarbazole base skeleton in formula (TA-2a) is H or a carbazole group which may be bonded to the indenocarbazole base skeleton via the 1, 2, 3 or 4 position or via the nitrogen atom, especially via the 3 position.

Preferred 4-spirocarbazole derivatives which are used as co-host materials together with the compounds of the invention are selected from the compounds of the following formula (TA-3):

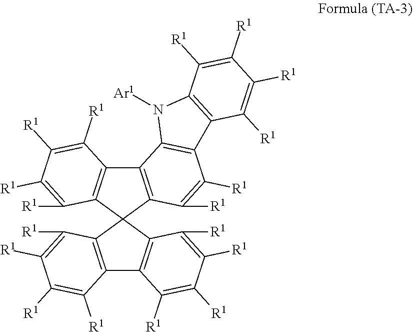

where Ar¹ and R¹ have the definitions listed above. Preferred embodiments of the Ar¹ group are the above-listed structures R¹-1 to R¹-79, more preferably R¹-1 to R¹-51.

A preferred embodiment of the compounds of the formula (TA-3) is the compounds of the following formula (TA-3a):

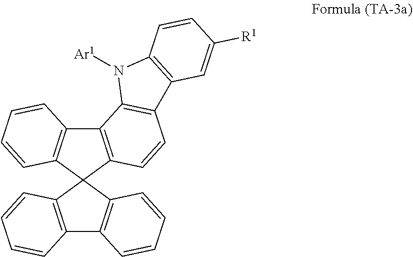

where Ar¹ and R¹ have the definitions listed above. Preferred embodiments of the Ar¹ group are the above-listed structures R¹-1 to R¹-79, more preferably R¹—I to R¹-51.

Preferred lactams which are used as co-host materials together with the compounds of the invention are selected from the compounds of the following formula (LAC-1):

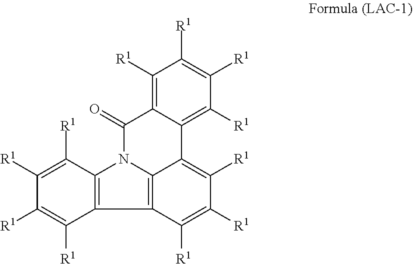

where R has the definitions listed above.

A preferred embodiment of the compounds of the formula (LAC-1) is the compounds of the following formula (LAC-1a):

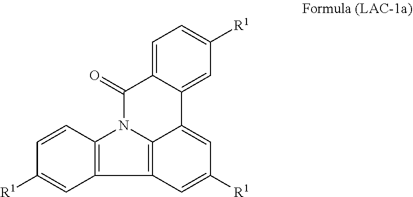

where R¹ has the definitions given above. R¹ is preferably the same or different at each instance and is H or an aromatic or heteroaromatic ring system which has 5 to 40 aromatic ring atoms and may be substituted by one or more R² radicals, where R² may have the definition given above, especially for formula (I). Most preferably, the R¹ substituents are selected from the group consisting of H and an aromatic or heteroaromatic ring system which has 6 to 18 aromatic ring atoms, preferably 6 to 13 aromatic ring atoms, and may be substituted in each case by one or more nonaromatic R² radicals, but is preferably unsubstituted. Examples of suitable R¹ substituents are selected from the group consisting of phenyl, ortho-, meta- or para-biphenyl, terphenyl, especially branched terphenyl, quaterphenyl, especially branched quaterphenyl, 1-, 2-, 3- or 4-fluorenyl, 1-, 2-, 3- or 4-spirobifluorenyl, pyridyl, pyrimidinyl, 1-, 2-, 3- or 4-dibenzofuranyl, 1-, 2-, 3- or 4-dibenzothienyl and 1-, 2-, 3- or 4-carbazolyl, each of which may be substituted by one or more R² radicals, but are preferably unsubstituted. Suitable R¹ structures are the same structures as depicted above for R-1 to R-79, more preferably R¹-1 to R¹-51.

It may also be preferable to use a plurality of different matrix materials as a mixture, especially at least one electron-conducting matrix material and at least one hole-conducting matrix material. Preference is likewise given to the use of a mixture of a charge-transporting matrix material and an electrically inert matrix material having no significant involvement, if any, in the charge transport, as described, for example, in WO 2010/108579.

It is further preferable to use a mixture of two or more triplet emitters together with a matrix. In this case, the triplet emitter having the shorter-wave emission spectrum serves as co-matrix for the triplet emitter having the longer-wave emission spectrum.

More preferably, it is possible to use a compound of the invention comprising structures of formula (I), in a preferred embodiment, as matrix material in an emission layer of an organic electronic device, especially in an organic electroluminescent device, for example in an OLED or OLEC. In this case, the matrix material containing compound comprising structures of formula (I) or the preferred embodiments recited above and hereinafter is present in the electronic device in combination with one or more dopants, preferably phosphorescent dopants.

The proportion of the matrix material in the emitting layer in this case is between 50.0% and 99.9% by volume, preferably between 80.0% and 99.5% by volume, and more preferably between 92.0% and 99.5% by volume for fluorescent emitting layers and between 85.0% and 97.0% by volume for phosphorescent emitting layers.

Correspondingly, the proportion of the dopant is between 0.1% and 50.0% by volume, preferably between 0.5% and 20.0% by volume, and more preferably between 0.5% and 8.0% by volume for fluorescent emitting layers and between 3.0% and 15.0% by volume for phosphorescent emitting layers.

An emitting layer of an organic electroluminescent device may also comprise systems comprising a plurality of matrix materials (mixed matrix systems) and/or a plurality of dopants. In this case too, the dopants are generally those materials having the smaller proportion in the system and the matrix materials are those materials having the greater proportion in the system. In individual cases, however, the proportion of a single matrix material in the system may be less than the proportion of a single dopant.

In a further preferred embodiment of the invention, the compound comprising structures of formula (I) or the preferred embodiments recited above and below are used as a component of mixed matrix systems. The mixed matrix systems preferably comprise two or three different matrix materials, more preferably two different matrix materials. Preferably, in this case, one of the two materials is a material having hole-transporting properties and the other material is a material having electron-transporting properties. The desired electron-transporting and hole-transporting properties of the mixed matrix components may, however, also be combined mainly or entirely in a single mixed matrix component, in which case the further mixed matrix component(s) fulfil(s) other functions. The two different matrix materials may be present in a ratio of 1:50 to 1:1, preferably 1:20 to 1:1, more preferably 1:10 to 1:1 and most preferably 1:4 to 1:1. Preference is given to using mixed matrix systems in phosphorescent organic electroluminescent devices. One source of more detailed information about mixed matrix systems is the application WO 2010/108579.

The present invention further provides an electronic device, preferably an organic electroluminescent device, comprising one or more compounds of the invention and/or at least one oligomer, polymer or dendrimer of the invention in one or more electron-conducting layers, as electron-conducting compound.

Preferred cathodes are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag, in which case combinations of the metals such as Mg/Ag, Ca/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). Likewise useful for this purpose are organic alkali metal complexes, e.g. Liq (lithium quinolinate). The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes has to be transparent or partly transparent in order to enable either the irradiation of the organic material (O-SC) or the emission of light (OLED/PLED, O-laser). Preferred anode materials here are conductive mixed metal oxides. Particular preference is given to indium tin oxide (ITO) or indium zinc oxide (IZO). Preference is further given to conductive doped organic materials, especially conductive doped polymers, for example PEDOT, PANI or derivatives of these polymers. It is further preferable when a p-doped hole transport material is applied to the anode as hole injection layer, in which case suitable p-dopants are metal oxides, for example MoO₃ or WO₃, or (per)fluorinated electron-deficient aromatic systems. Further suitable p-dopants are HAT-CN (hexacyanohexaazatriphenylene) or the compound NPD9 from Novaled. Such a layer simplifies hole injection into materials having a low HOMO, i.e. a large HOMO in terms of magnitude.

In the further layers, it is generally possible to use any materials as used according to the prior art for the layers, and the person skilled in the art is able, without exercising inventive skill, to combine any of these materials with the materials of the invention in an electronic device.

The device is correspondingly (according to the application) structured, contact-connected and finally hermetically sealed, since the lifetime of such devices is severely shortened in the presence of water and/or air.

Additionally preferred is an electronic device, especially an organic electroluminescent device, which is characterized in that one or more layers are coated by a sublimation process. In this case, the materials are applied by vapour deposition in vacuum sublimation systems at an initial pressure of typically less than 10⁻⁵ mbar, preferably less than 10⁻⁶ mbar. It is also possible that the initial pressure is even lower or even higher, for example less than 10⁻⁷ mbar.

Preference is likewise given to an electronic device, especially an organic electroluminescent device, which is characterized in that one or more layers are coated by the OVPD (organic vapour phase deposition) method or with the aid of a carrier gas sublimation. In this case, the materials are applied at a pressure between 10⁻⁵ mbar and 1 bar. A special case of this method is the OVJP (organic vapour jet printing) method, in which the materials are applied directly by a nozzle and thus structured (for example M. S. Arnold et al., Appl. Phys. Lett. 2008, 92, 053301).

Preference is additionally given to an electronic device, especially an organic electroluminescent device, which is characterized in that one or more layers are produced from solution, for example by spin-coating, or by any printing method, for example screen printing, flexographic printing, offset printing or nozzle printing, but more preferably LITI (light-induced thermal imaging, thermal transfer printing) or inkjet printing. For this purpose, soluble compounds are needed, which are obtained, for example, through suitable substitution.

The electronic device, especially the organic electroluminescent device, can also be produced as a hybrid system by applying one or more layers from solution and applying one or more other layers by vapour deposition. For example, it is possible to apply an emitting layer comprising a compound of the invention comprising structures of formula (I) and a matrix material from solution, and to apply a hole blocker layer and/or an electron transport layer thereto by vapour deposition under reduced pressure.

These methods are known in general terms to those skilled in the art and can be applied without difficulty to electronic devices, especially organic electroluminescent devices comprising compounds of the invention comprising structures of formula (I) or the above-detailed preferred embodiments.

The electronic devices of the invention, especially organic electroluminescent devices, are notable for one or more of the following surprising advantages over the prior art:
1. Electronic devices, especially organic electroluminescent devices, comprising compounds, oligomers, polymers or dendrimers having structures of formula (I) or the preferred embodiments recited above and hereinafter, especially as electron-conducting materials, have a very good lifetime.
2. Electronic devices, especially organic electroluminescent devices, comprising compounds, oligomers, polymers or dendrimers having structures of formula (I) or the preferred embodiments recited above and hereinafter, as electron-conducting materials have excellent efficiency. More particularly, efficiency is much higher compared to analogous compounds containing no structural unit of formula (I).
3. The compounds, oligomers, polymers or dendrimers of the invention having structures of formula (I) or the preferred embodiments recited above and hereinafter exhibit very high stability and lead to compounds having a very long lifetime.
4. With compounds, oligomers, polymers or dendrimers having structures of formula (I) or the preferred embodiments recited above and hereinafter, it is possible to avoid the formation of optical loss channels in electronic devices, especially organic electroluminescent devices. As a result, these devices feature a high PL efficiency and hence high EL efficiency of emitters, and excellent energy transmission of the matrices to dopants.
5. The use of compounds, oligomers, polymers or dendrimers having structures of formula (I) or the preferred embodiments recited above and hereinafter in layers of electronic devices, especially organic electroluminescent devices, leads to high mobility of the electron conductor structures.
6. Compounds, oligomers, polymers or dendrimers having structures of formula (I) or the preferred embodiments recited above and below feature excellent thermal stability, and compounds having a molar mass of less than about 1200 g/mol have good sublimability.
7. Compounds, oligomers, polymers or dendrimers having structures of formula (I) or the preferred embodiments recited above and hereinafter have excellent glass film formation.
8. Compounds, oligomers, polymers or dendrimers having structures of formula (I) or the preferred embodiments recited above and hereinafter form very good films from solutions.
9. The compounds, oligomers, polymers or dendrimers comprising structures of formula (I) or the preferred embodiments recited above and hereinafter have a surprisingly high triplet level T₁, this being particularly true of compounds which are used as electron-conducting materials.

These abovementioned advantages are not accompanied by a deterioration in the further electronic properties.

The compounds and mixtures of the invention are suitable for use in an electronic device. An electronic device is understood to mean a device containing at least one layer containing at least one organic compound. The component may also comprise inorganic materials or else layers formed entirely from inorganic materials.

The present invention therefore further provides for the use of the compounds or mixtures of the invention in an electronic device, especially in an organic electroluminescent device.

The present invention still further provides for the use of a compound of the invention and/or of an oligomer, polymer or dendrimer of the invention in an electronic device as hole blocker material, electron injection material and/or electron transport material.

The present invention still further provides an electronic device comprising at least one of the above-detailed compounds or mixtures of the invention. In this case, the preferences detailed above for the compound also apply to the electronic devices.

In a further embodiment of the invention, the organic electroluminescent device of the invention does not contain any separate hole injection layer and/or hole transport layer and/or hole blocker layer and/or electron transport layer, meaning that the emitting layer directly adjoins the hole injection layer or the anode, and/or the emitting layer directly adjoins the electron transport layer or the electron injection layer or the cathode, as described, for example, in WO 2005/053051. It is additionally possible to use a metal complex identical or similar to the metal complex in the emitting layer as hole transport or hole injection material directly adjoining the emitting layer, as described, for example, in WO 2009/030981.

In addition, it is possible to use the compounds of the invention in a hole blocker or electron transport layer. This is especially true of compounds of the invention which do not have a carbazole structure. These may preferably also be substituted by one or more further electron-transporting groups, for example benzimidazole groups.

In the further layers of the organic electroluminescent device of the invention, it is possible to use any materials as typically used according to the prior art. The person skilled in the art is therefore able, without exercising inventive skill, to use any materials known for organic electroluminescent devices in combination with the inventive compounds of formula (I) or according to the preferred embodiments.

The compounds of the invention generally have very good properties on use in organic electroluminescent devices. Especially in the case of use of the compounds of the invention in organic electroluminescent devices, the lifetime is significantly better compared to similar compounds according to the prior art. At the same time, the further properties of the organic electroluminescent device, especially the efficiency and voltage, are likewise better or at least comparable.

It should be pointed out that variations of the embodiments described in the present invention are covered by the scope of this invention. Any feature disclosed in the present invention may, unless this is explicitly ruled out, be exchanged for alternative features which serve the same purpose or an equivalent or similar purpose. Thus, any feature disclosed in the present invention, unless stated otherwise, should be considered as an example of a generic series or as an equivalent or similar feature.

All features of the present invention may be combined with one another in any manner, unless particular features and/or steps are mutually exclusive. This is especially true of preferred features of the present invention. Equally, features of non-essential combinations may be used separately (and not in combination).

It should also be pointed out that many of the features, and especially those of the preferred embodiments of the present invention, are themselves inventive and should not be regarded merely as some of the embodiments of the present invention. For these features, independent protection may be sought in addition to or as an alternative to any currently claimed invention.

The technical teaching disclosed with the present invention may be abstracted and combined with other examples.

The invention is illustrated in detail by the examples which follow, without any intention of restricting it thereby.

The person skilled in the art will be able to use the details given, without exercising inventive skill, to produce further electronic devices of the invention and hence to execute the invention over the entire scope claimed.

### EXAMPLES

The syntheses which follow, unless stated otherwise, are conducted under a protective gas atmosphere in dried solvents. The solvents and reagents can be purchased, for example, from Sigma-ALDRICH or ABCR. For the compounds known from the literature, the corresponding CAS numbers are also reported in each case.

### Synthesis Examples

### a) 6-Bromo-2-fluoro-2′-methoxybiphenyl

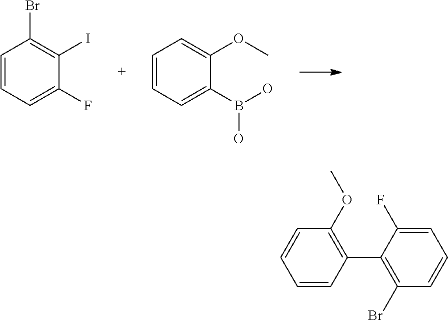 [SMILES: Fc1cccc(Br)c1I ; COc1ccccc1B(O)O ; COc1ccccc1-c1c(F)cccc1Br]

200 g (664 mmol) of 1-bromo-3-fluoro-2-iodobenzene, 101 g (664 mmol) of 2-methoxyphenylboronic acid and 137.5 g (997 mmol) of sodium tetraborate are dissolved in 1000 ml of THF and 600 ml of water, and degassed. 9.3 g (13.3 mmol) of bis(triphenylphosphine)palladium(II) chloride and 1 g (20 mmol) of hydrazinium hydroxide are added. The reaction mixture is then stirred under a protective gas atmosphere at 70° C. for 48 h. The cooled solution is supplemented with toluene, washed repeatedly with water, dried and concentrated. The product is purified via column chromatography on silica gel with toluene/heptane (1:2). Yield: 155 g (553 mmol), 83% of theory.

The following compounds are prepared in an analogous manner:

| | Reactant 1 | Reactant 2 | Product | Yield |
|---|---|---|---|---|
| a1 | 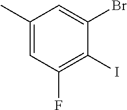 | 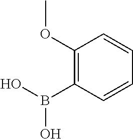 | 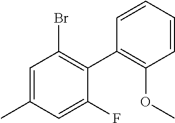 | 77% |
| | [1000576-09-9] | | | |

### b) 6′-Bromo-2′-fluorobiphenyl-2-ol

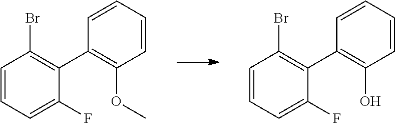 [SMILES: COc1ccccc1-c1c(F)cccc1Br ; Oc1ccccc1-c1c(F)cccc1Br]

112 g (418 mmol) of 6-bromo-2-fluoro-2′-methoxybiphenyl are dissolved in 2 I of dichloromethane and cooled to 5° C. 41.01 ml (431 mmol) of boron tribromide are added dropwise to this solution within 90 min, and stirring of the mixture continues overnight. The mixture is subsequently admixed gradually with water, and the organic phase is washed three times with water, dried over Na₂SO₄, concentrated by rotary evaporation and purified by chromatography. Yield: 104 g (397 mmol), 98% of theory.

The following compounds are prepared in an analogous manner:

| | Reactant 1 | Product | Yield |
|---|---|---|---|
| b1 | 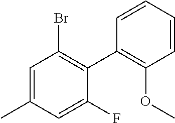 | 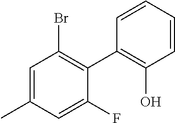 [SMILES: Cc1cc(F)c(-c2ccccc2O)c(Br)c1] | 92% |

### c) 1-Bromodibenzofuran

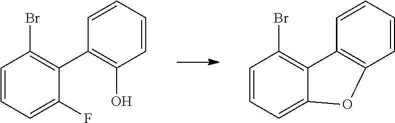 [SMILES: Oc1ccccc1-c1c(F)cccc1Br ; Brc1cccc2oc3ccccc3c12]

111 g (416 mmol) of 6′-bromo-2′-fluorobiphenyl-2-ol are dissolved in 2 I of DMF (max. 0.003% H₂O) SeccoSolv® and cooled to 5° C. 20 g (449 mmol) of sodium hydride (60% suspension in paraffin oil) are added to this solution in portions, once the addition has ended the mixture is stirred for 20 min, and then the mixture is heated to 100° C. for 45 min. After cooling, 500 ml of ethanol are added gradually to the mixture, which is concentrated completely by rotary evaporation and then purified by chromatography. Yield: 90 g (367 mmol), 88.5% of theory.

The following compounds are prepared in an analogous manner:

| | Reactant 1 | Product | Yield |
|---|---|---|---|
| c1 | 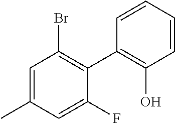 [SMILES: Cc1cc(F)c(-c2ccccc2O)c(Br)c1] | 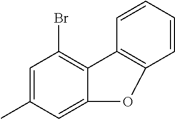 | 81% |

### d) 1-Bromo-8-iododibenzofuran

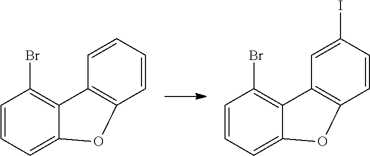 [SMILES: Brc1cccc2oc3ccccc3c12 ; Brc1cccc2oc3ccc(I)cc3c12]

20 g (80 mmol) of 1-bromodibenzofuran, 2.06 g (40.1 mmol) of iodine, 3.13 g (17.8 mmol) of iodic acid, 80 ml of acetic acid, 5 ml of sulphuric acid, 5 ml of water and 2 ml of chloroform are stirred at 65° for 3 h. After cooling, the mixture is admixed with water, and the precipitated solids are filtered off with suction and washed three times with water. The residue is recrystallized from toluene and from dichloromethane/heptane. The yield is 25.6 g (68 mmol), corresponding to 85% of theory.

The following compounds are prepared in an analogous manner:

| | Reactant 1 | Product | Yield |
|---|---|---|---|
| d1 | 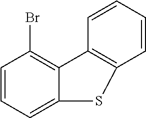 [SMILES: Brc1cccc2sc3ccccc3c12] | 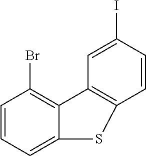 [SMILES: Brc1cccc2sc3ccc(I)cc3c12] | 81% |
| | [65642-94-6] | | |

### e) Dibenzofuran-1-boronic acid

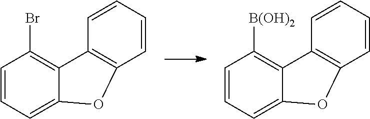 [SMILES: Brc1cccc2oc3ccccc3c12 ; OB(O)c1cccc2oc3ccccc3c12]

180 g (728 mmol) of 1-bromodibenzofuran are dissolved in 1500 ml of dry THF and cooled to −78° C. At this temperature, 305 ml (764 mmol/2.5 M in hexane) of n-butyllithium are added within about 5 min and then the mixture is stirred at −78° C. for a further 2.5 h. At this temperature, 151 g (1456 mmol) of trimethyl borate are added very rapidly and the reaction is allowed to come gradually to room temperature (about 18 h). The reaction solution is washed with water and the precipitated solids and the organic phase are subjected to azeotropic drying with toluene. The crude product is extracted while stirring from toluene/methylene chloride at about 40° C. and filtered off with suction. Yield: 146 g (690 mmol), 95% of theory.

The following compounds are prepared in an analogous manner:

| | Reactant 1 | Product | Yield |
|---|---|---|---|
| e1 | 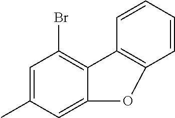 | 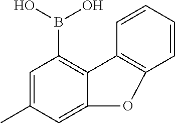 [SMILES: Cc1cc(B(O)O)c2c(c1)oc1ccccc12] | 81% |
| e2 | 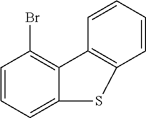 [SMILES: Brc1cccc2sc3ccccc3c12] | 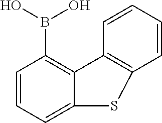 [SMILES: OB(O)c1cccc2sc3ccccc3c12] | 73% |
| | [65642-94-6] | | |
| e3 | 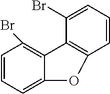 [SMILES: Brc1cccc2oc3cccc(Br)c3c12] | 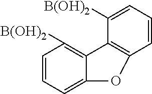 [SMILES: OB(O)c1cccc2oc3cccc(B(O)O)c3c12] | 63% |
| | [617707-29-6] | | |
| e4 | 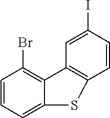 [SMILES: Brc1cccc2sc3ccc(I)cc3c12] | 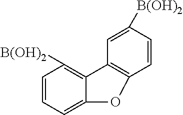 [SMILES: OB(O)c1ccc2oc3cccc(B(O)O)c3c2c1] | 74% |
| e5 | 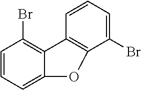 [SMILES: Brc1cccc2c1oc1cccc(Br)c12] | 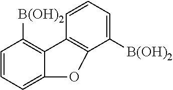 [SMILES: OB(O)c1cccc2c1oc1cccc(B(O)O)c12] | 79% |
| | [617707-26-3] | | |
| e6 | 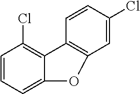 [SMILES: Clc1ccc2c(c1)oc1cccc(Cl)c12] | 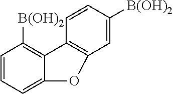 [SMILES: OB(O)c1ccc2c(c1)oc1cccc(B(O)O)c12] | 47% |
| | [94538-02-0] | | |

### f) 4-Biphenyl-4-yl-2-chloroquinazoline

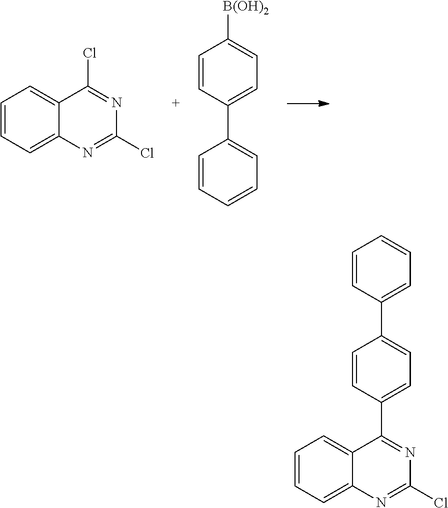 [SMILES: Clc1nc(Cl)c2ccccc2n1 ; OB(O)c1ccc(-c2ccccc2)cc1 ; Clc1nc(-c2ccc(-c3ccccc3)cc2)c2ccccc2n1]

13 g (70 mmol) of biphenyl-4-boronic acid, 13.8 g (70 mmol) of 2,4-dichloroquinazoline and 14.7 g (139 mmol) of sodium carbonate are suspended in 200 ml of toluene, 52 ml of ethanol and 100 ml of water. 800 mg (0.69 mmol) of tetrakisphenylphosphinepalladium(0) are added to this suspension, and the reaction mixture is heated under reflux for 16 h. After cooling, the organic phase is removed, filtered through silica gel, washed three times with 200 ml of water and then concentrated to dryness. The residue is recrystallized from heptane/dichloromethane. The yield is 13 g (41 mmol), corresponding to 59% of theory.

The following compound is obtained in an analogous manner:

| | Reactant 1 | Reactant 2 | Product | Yield |
|---|---|---|---|---|
| f1 | 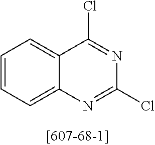 [SMILES: Clc1nc(Cl)c2ccccc2n1] | 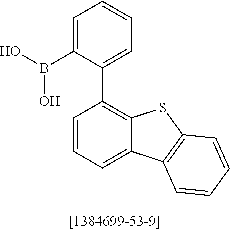 [SMILES: OB(O)c1ccccc1-c1cccc2c1sc1ccccc12] | 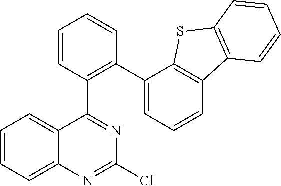 [SMILES: Clc1nc(-c2ccccc2-c2cccc3c2sc2ccccc23)c2ccccc2n1] | 76% |
| f2 | 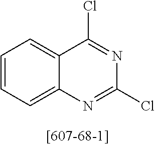 [SMILES: Clc1nc(Cl)c2ccccc2n1] | 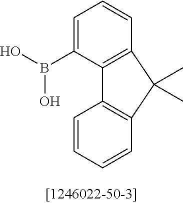 [SMILES: CC1(C)c2ccccc2-c2c(B(O)O)cccc21] | 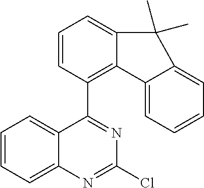 [SMILES: CC1(C)c2ccccc2-c2c(-c3nc(Cl)nc4ccccc34)cccc21] | 75% |
| f3 | 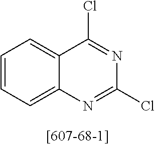 [SMILES: Clc1nc(Cl)c2ccccc2n1] | 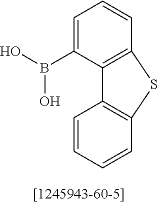 [SMILES: OB(O)c1cccc2sc3ccccc3c12] | 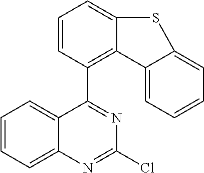 [SMILES: Clc1nc(-c2cccc3sc4ccccc4c23)c2ccccc2n1] | 73% |
| f4 | 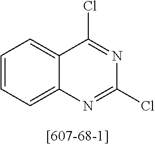 [SMILES: Clc1nc(Cl)c2ccccc2n1] | 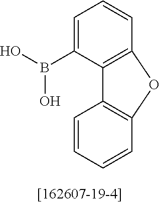 [SMILES: OB(O)c1cccc2oc3ccccc3c12] | 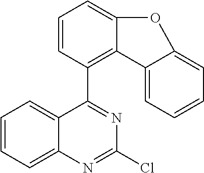 [SMILES: Clc1nc(-c2cccc3oc4ccccc4c23)c2ccccc2n1] | 78% |
| f5 | 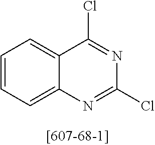 [SMILES: Clc1nc(Cl)c2ccccc2n1] | 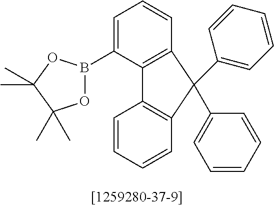 [SMILES: CC1(C)OB(c2cccc3c2-c2ccccc2C3(c2ccccc2)c2ccccc2)OC1(C)C] | 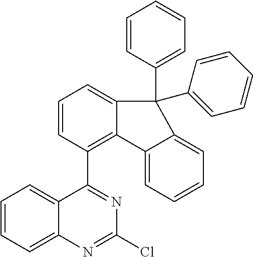 [SMILES: Clc1nc(-c2cccc3c2-c2ccccc2C3(c2ccccc2)c2ccccc2)c2ccccc2n1] | 76% |
| f6 | 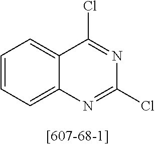 [SMILES: Clc1nc(Cl)c2ccccc2n1] | 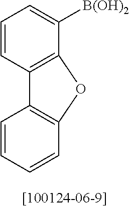 [SMILES: OB(O)c1cccc2c1oc1ccccc12] | 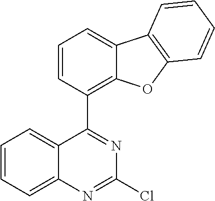 [SMILES: Clc1nc(-c2cccc3c2oc2ccccc23)c2ccccc2n1] | 77% |
| f7 | 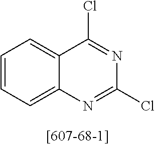 [SMILES: Clc1nc(Cl)c2ccccc2n1] | 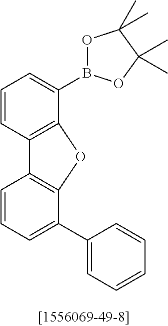 [SMILES: CC1(C)OB(c2cccc3c2oc2c(-c4ccccc4)cccc23)OC1(C)C] | 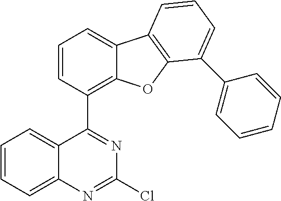 [SMILES: Clc1nc(-c2cccc3c2oc2c(-c4ccccc4)cccc23)c2ccccc2n1] | 72% |
| f8 | 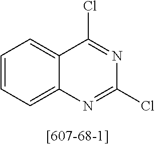 [SMILES: Clc1nc(Cl)c2ccccc2n1] | 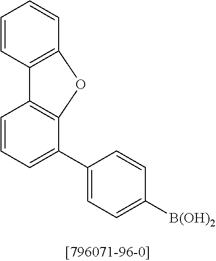 [SMILES: OB(O)c1ccc(-c2cccc3c2oc2ccccc23)cc1] | 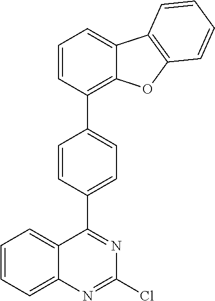 [SMILES: Clc1nc(-c2ccc(-c3cccc4c3oc3ccccc34)cc2)c2ccccc2n1] | 73% |
| f9 | 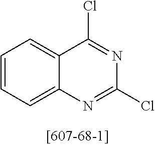 [SMILES: Clc1nc(Cl)c2ccccc2n1] | 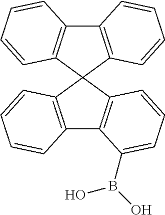 [SMILES: OB(O)c1cccc2c1-c1ccccc1C21c2ccccc2-c2ccccc21] | 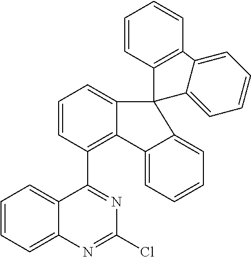 [SMILES: Clc1nc(-c2cccc3c2-c2ccccc2C32c3ccccc3-c3ccccc32)c2ccccc2n1] | 72% |

### g) 2-Dibenzofuran-1-yl-4-phenylquinazoline

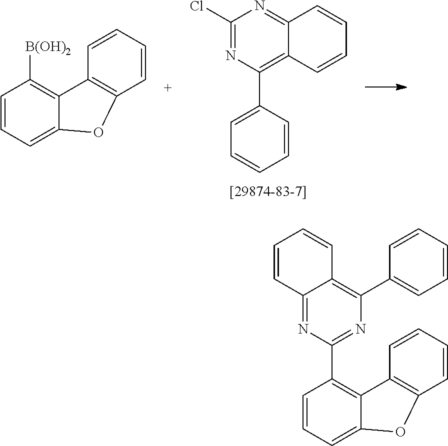 [SMILES: OB(O)c1cccc2oc3ccccc3c12 ; Clc1nc(-c2ccccc2)c2ccccc2n1 ; c1ccc(-c2nc(-c3cccc4oc5ccccc5c34)nc3ccccc23)cc1]

23 g (110.0 mmol) of dibenzofuran-1-boronic acid, 29.5 g (110.0 mmol) of 2-chloro-4-phenylquinazoline and 26 g (210.0 mmol) of sodium carbonate are suspended in 500 ml of ethylene glycol diamine ether and 500 ml of water. Added to this suspension are 913 mg (3.0 mmol) of tri-o-tolylphosphine and then 112 mg (0.5 mmol) of palladium(II) acetate, and the reaction mixture is heated under reflux for 16 h. After cooling, the organic phase is removed, filtered through silica gel, washed three times with 200 ml of water and then concentrated to dryness. The residue is recrystallized from toluene and from dichloromethane/heptane. The yield is 32 g (86 mmol), corresponding to 80% of theory.

The following compounds are prepared in an analogous manner:

| | Reactant 1 | Reactant 2 | Product | Yield |
|---|---|---|---|---|
| g1 | 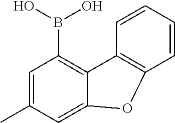 [SMILES: Cc1cc(B(O)O)c2c(c1)oc1ccccc12] | 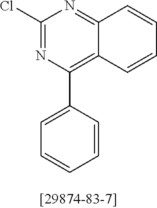 [SMILES: Clc1nc(-c2ccccc2)c2ccccc2n1] | 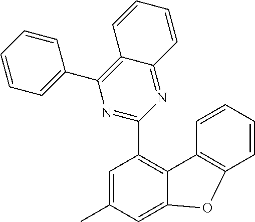 [SMILES: Cc1cc(-c2nc(-c3ccccc3)c3ccccc3n2)c2c(c1)oc1ccccc12] | 78% |
| g2 | 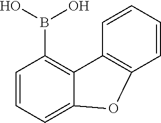 [SMILES: OB(O)c1cccc2oc3ccccc3c12] | 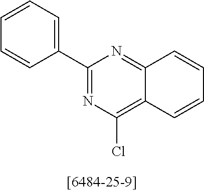 [SMILES: Clc1nc(-c2ccccc2)nc2ccccc12] | 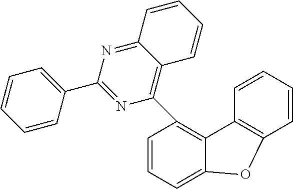 [SMILES: c1ccc(-c2nc(-c3cccc4oc5ccccc5c34)c3ccccc3n2)cc1] | 70% |
| g3 | 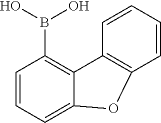 [SMILES: OB(O)c1cccc2oc3ccccc3c12] | 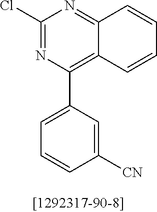 [SMILES: N#Cc1cccc(-c2nc(Cl)nc3ccccc23)c1] | 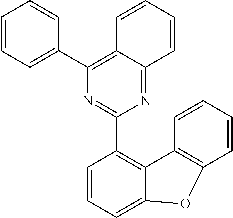 [SMILES: c1ccc(-c2nc(-c3cccc4oc5ccccc5c34)nc3ccccc23)cc1] | 71% |
| g4 | 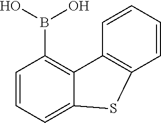 [SMILES: OB(O)c1cccc2sc3ccccc3c12] | 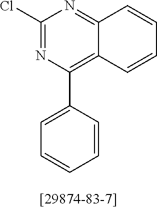 [SMILES: Clc1nc(-c2ccccc2)c2ccccc2n1] | 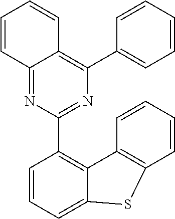 [SMILES: c1ccc(-c2nc(-c3cccc4sc5ccccc5c34)nc3ccccc23)cc1] | 77% |
| g5 | 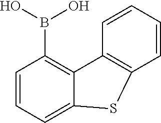 [SMILES: OB(O)c1cccc2sc3ccccc3c12] | 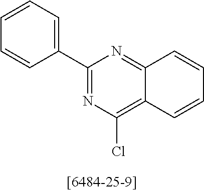 [SMILES: Clc1nc(-c2ccccc2)nc2ccccc12] | 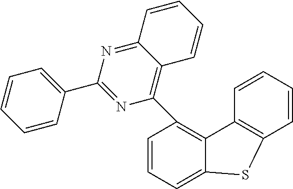 [SMILES: c1ccc(-c2nc(-c3cccc4sc5ccccc5c34)c3ccccc3n2)cc1] | 76% |
| g6 | 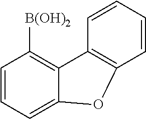 [SMILES: OB(O)c1cccc2oc3ccccc3c12] | 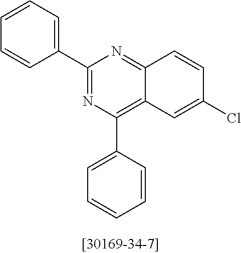 [SMILES: Clc1ccc2nc(-c3ccccc3)nc(-c3ccccc3)c2c1] | 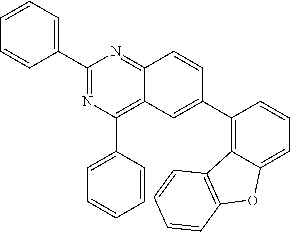 [SMILES: c1ccc(-c2nc(-c3ccccc3)c3cc(-c4cccc5oc6ccccc6c45)ccc3n2)cc1] | 71% |

### h) 2-(8-Bromodibenzofuran-1-yl)-4-phenylquinazoline

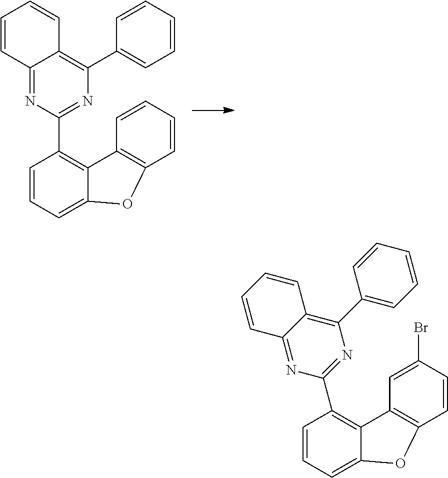 [SMILES: c1ccc(-c2nc(-c3cccc4oc5ccccc5c34)nc3ccccc23)cc1 ; Brc1ccc2oc3cccc(-c4nc(-c5ccccc5)c5ccccc5n4)c3c2c1]

70.6 g (190.0 mmol) of 2-dibenzofuran-1-yl-4-phenylquinazoline are suspended in 2000 ml of acetic acid (100%) and 2000 ml of sulphuric acid (95-98%). 34 g (190 mmol) of NBS added to the suspension and the mixture is stirred in the dark for 2 hours. Thereafter, water/ice is added and solids are removed and washed with ethanol. The residue is recrystallized from toluene. The yield is 59 g (130 mmol), corresponding to 69% of theory.

In the case of the thiophene derivatives, nitrobenzene is used rather than sulphuric acid and elemental bromine in place of NBS:

The following compounds are prepared in an analogous manner:

| | Reactant 1 | Product | Yield |
|---|---|---|---|
| h1 | 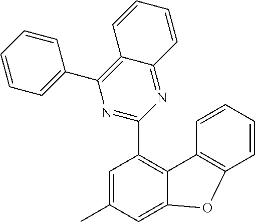 [SMILES: Cc1cc(-c2nc(-c3ccccc3)c3ccccc3n2)c2c(c1)oc1ccccc12] | 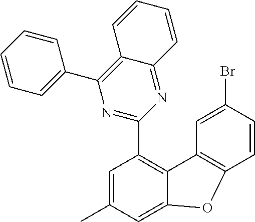 [SMILES: Cc1cc(-c2nc(-c3ccccc3)c3ccccc3n2)c2c(c1)oc1ccc(Br)cc12] | 70% |
| h2 | 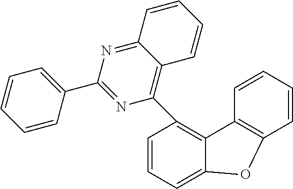 [SMILES: c1ccc(-c2nc(-c3cccc4oc5ccccc5c34)c3ccccc3n2)cc1] | 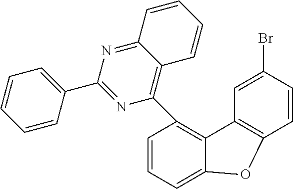 [SMILES: Brc1ccc2oc3cccc(-c4nc(-c5ccccc5)nc5ccccc45)c3c2c1] | 73% |
| h3 | 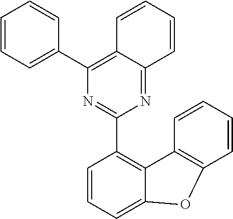 [SMILES: c1ccc(-c2nc(-c3cccc4oc5ccccc5c34)nc3ccccc23)cc1] | 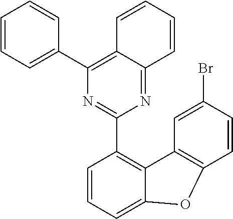 [SMILES: Brc1ccc2oc3cccc(-c4nc(-c5ccccc5)c5ccccc5n4)c3c2c1] | 71% |
| h4 | 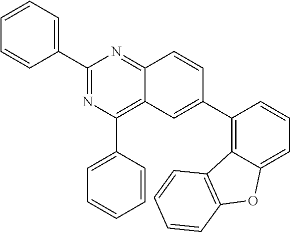 [SMILES: c1ccc(-c2nc(-c3ccccc3)c3cc(-c4cccc5oc6ccccc6c45)ccc3n2)cc1] | 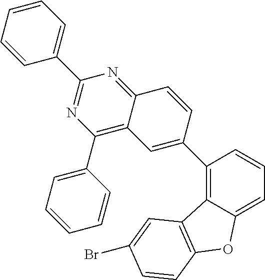 [SMILES: Brc1ccc2oc3cccc(-c4ccc5nc(-c6ccccc6)nc(-c6ccccc6)c5c4)c3c2c1] | 69% |

### j) 2-Dibenzofuran-1-yl-4,6diphenyl-[1,3,5]triazine

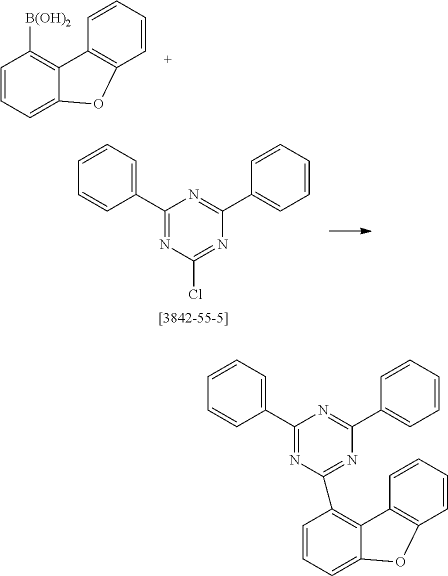

23 g (110.0 mmol) of dibenzofuran-1-boronic acid, 29.5 g (110.0 mmol) of 2-chloro-4,6-diphenyl-1,3,5-triazine and 21 g (210.0 mmol) of sodium carbonate are suspended in 500 ml of ethylene glycol diamine ether and 500 ml of water. Added to this suspension are 913 mg (3.0 mmol) of tri-o-tolylphosphine and then 112 mg (0.5 mmol) of palladium(II) acetate, and the reaction mixture is heated under reflux for 16 h. After cooling, the organic phase is removed, filtered through silica gel, washed three times with 200 ml of water and then concentrated to dryness. The residue is recrystallized from toluene and from dichloromethane/heptane. The yield is 37 g (94 mmol), corresponding to 87% of theory.

The following compounds are prepared in an analogous manner:

| | Reactant 1 | Reactant 2 | Product | Yield |
|---|---|---|---|---|
| j1 | 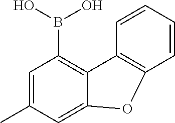 [SMILES: Cc1cc(B(O)O)c2c(c1)oc1ccccc12] | 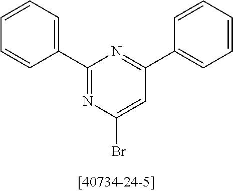 [SMILES: Brc1cc(-c2ccccc2)nc(-c2ccccc2)n1] | 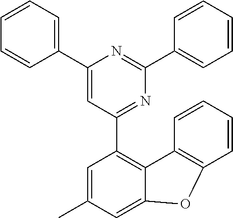 [SMILES: Cc1cc(-c2cc(-c3ccccc3)nc(-c3ccccc3)n2)c2c(c1)oc1ccccc12] | 73% |
| j2 | 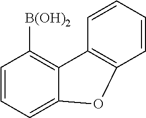 [SMILES: OB(O)c1cccc2oc3ccccc3c12] | 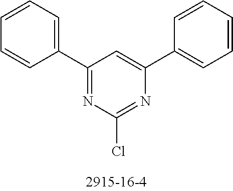 [SMILES: Clc1nc(-c2ccccc2)cc(-c2ccccc2)n1] | 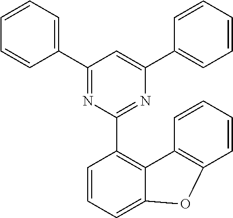 [SMILES: c1ccc(-c2cc(-c3ccccc3)nc(-c3cccc4oc5ccccc5c34)n2)cc1] | 75% |
| j3 | 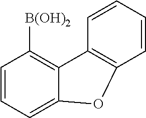 [SMILES: OB(O)c1cccc2oc3ccccc3c12] | 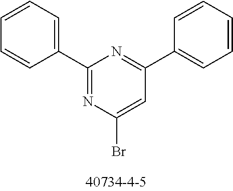 [SMILES: Brc1cc(-c2ccccc2)nc(-c2ccccc2)n1] | 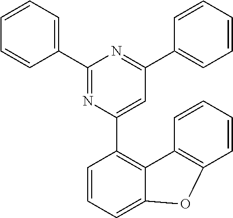 [SMILES: c1ccc(-c2cc(-c3cccc4oc5ccccc5c34)nc(-c3ccccc3)n2)cc1] | 76% |
| j4 | 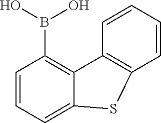 [SMILES: OB(O)c1cccc2sc3ccccc3c12] | 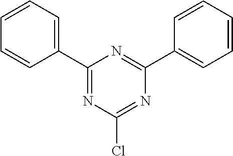 [SMILES: Clc1nc(-c2ccccc2)nc(-c2ccccc2)n1] | 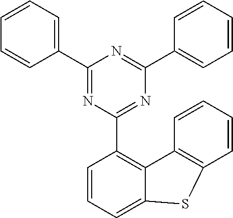 [SMILES: c1ccc(-c2nc(-c3ccccc3)nc(-c3cccc4sc5ccccc5c34)n2)cc1] | 63% |
| j5 | 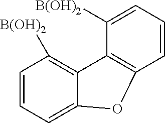 [SMILES: OB(O)c1cccc2oc3cccc(B(O)O)c3c12] | 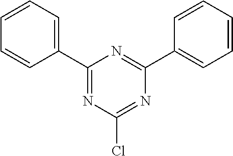 [SMILES: Clc1nc(-c2ccccc2)nc(-c2ccccc2)n1] | 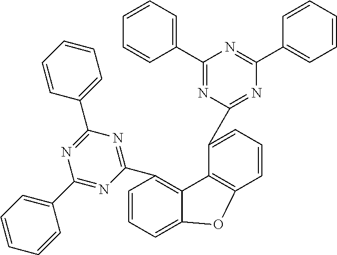 [SMILES: c1ccc(-c2nc(-c3ccccc3)nc(-c3cccc4oc5cccc(-c6nc(-c7ccccc7)nc(-c7ccccc7)n6)c5c34)n2)cc1] | 57% |
| j6 | 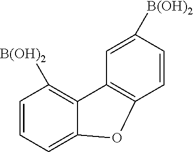 [SMILES: OB(O)c1ccc2oc3cccc(B(O)O)c3c2c1] | 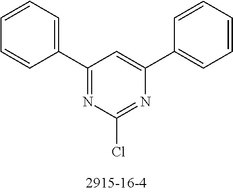 [SMILES: Clc1nc(-c2ccccc2)cc(-c2ccccc2)n1] | 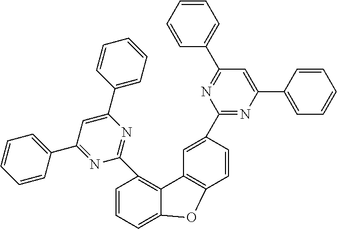 [SMILES: c1ccc(-c2cc(-c3ccccc3)nc(-c3ccc4oc5cccc(-c6nc(-c7ccccc7)cc(-c7ccccc7)n6)c5c4c3)n2)cc1] | 53% |
| j7 | 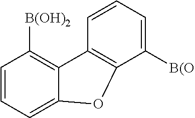 | 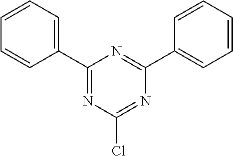 [SMILES: Clc1nc(-c2ccccc2)nc(-c2ccccc2)n1] | 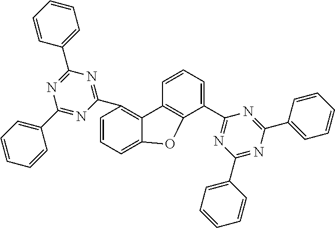 [SMILES: c1ccc(-c2nc(-c3ccccc3)nc(-c3cccc4c3oc3cccc(-c5nc(-c6ccccc6)nc(-c6ccccc6)n5)c34)n2)cc1] | 62% |
| j8 | 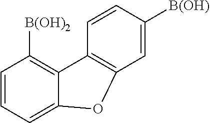 | 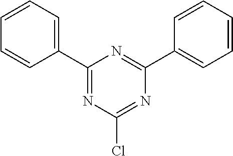 [SMILES: Clc1nc(-c2ccccc2)nc(-c2ccccc2)n1] | 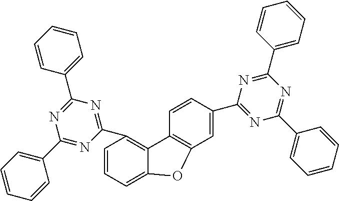 [SMILES: c1ccc(-c2nc(-c3ccccc3)nc(-c3ccc4c(c3)oc3cccc(-c5nc(-c6ccccc6)nc(-c6ccccc6)n5)c34)n2)cc1] | 64% |

### i) 2-(8-Bromodibenzofuran-1-yl)-4,6-diphenyl-[1,3,5]triazine

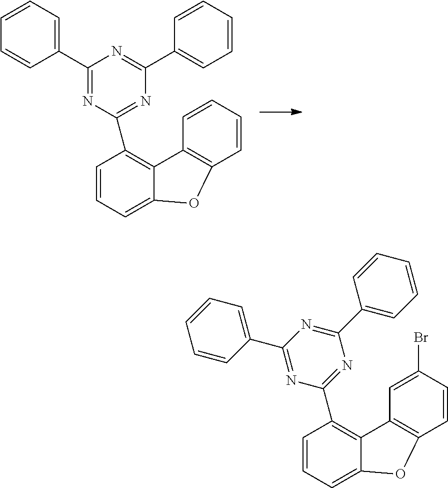 [SMILES: c1ccc(-c2nc(-c3ccccc3)nc(-c3cccc4oc5ccccc5c34)n2)cc1 ; Brc1ccc2oc3cccc(-c4nc(-c5ccccc5)nc(-c5ccccc5)n4)c3c2c1]

70 g (190.0 mmol) of 2-dibenzofuran-1-yl-4,6-diphenyl-[1,3,5]triazine are suspended in 2000 ml of acetic acid (100%) and 2000 ml of sulphuric acid (95-98%). 34 g (190 mmol) of NBS added to the suspension and the mixture is stirred in the dark for 2 hours. Thereafter, water/ice is added and solids are removed and washed with ethanol. The residue is recrystallized in toluene. The yield is 80 g (167 mmol), corresponding to 87% of theory.

The following compounds are prepared in an analogous manner:

| | Reactant 1 | Product | Yield |
|---|---|---|---|
| i1 | 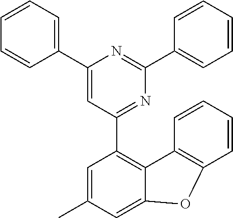 [SMILES: Cc1cc(-c2cc(-c3ccccc3)nc(-c3ccccc3)n2)c2c(c1)oc1ccccc12] | 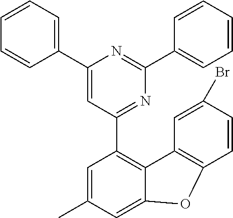 [SMILES: Cc1cc(-c2cc(-c3ccccc3)nc(-c3ccccc3)n2)c2c(c1)oc1ccc(Br)cc12] | 80% |
| i2 | 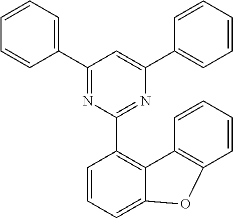 [SMILES: c1ccc(-c2cc(-c3ccccc3)nc(-c3cccc4oc5ccccc5c34)n2)cc1] | 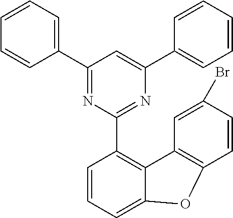 [SMILES: Brc1ccc2oc3cccc(-c4nc(-c5ccccc5)cc(-c5ccccc5)n4)c3c2c1] | 79% |
| i3 | 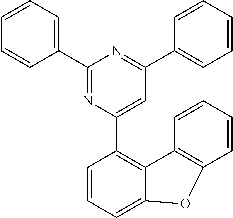 [SMILES: c1ccc(-c2cc(-c3cccc4oc5ccccc5c34)nc(-c3ccccc3)n2)cc1] | 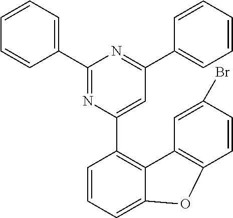 [SMILES: Brc1ccc2oc3cccc(-c4cc(-c5ccccc5)nc(-c5ccccc5)n4)c3c2c1] | 70% |

In the case of thiophene derivatives, nitrobenzene is used rather than sulphuric acid and elemental bromine in place of NBS:

| | Reactant 1 | Product | Yield |
|---|---|---|---|
| i4 | 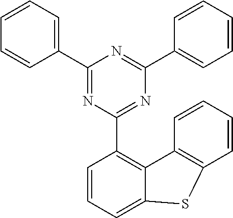 [SMILES: c1ccc(-c2nc(-c3ccccc3)nc(-c3cccc4sc5ccccc5c34)n2)cc1] | 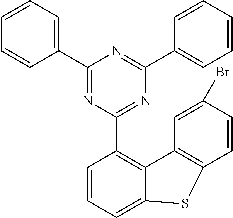 [SMILES: Brc1ccc2sc3cccc(-c4nc(-c5ccccc5)nc(-c5ccccc5)n4)c3c2c1] | 55% |
| i5 | 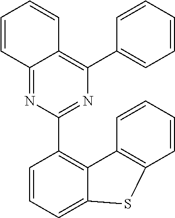 [SMILES: c1ccc(-c2nc(-c3cccc4sc5ccccc5c34)nc3ccccc23)cc1] | 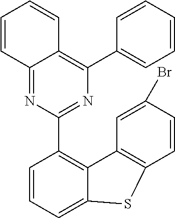 [SMILES: Brc1ccc2sc3cccc(-c4nc(-c5ccccc5)c5ccccc5n4)c3c2c1] | 62% |
| i6 | 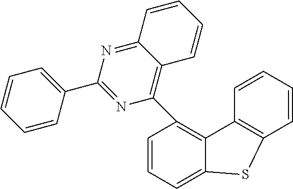 [SMILES: c1ccc(-c2nc(-c3cccc4sc5ccccc5c34)c3ccccc3n2)cc1] | 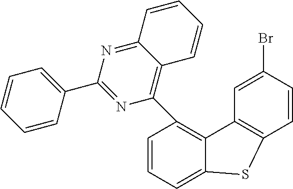 [SMILES: Brc1ccc2sc3cccc(-c4nc(-c5ccccc5)nc5ccccc45)c3c2c1] | 60% |

### k) 2,4-Diphenyl-6-[8-(4,4,5,5-tetramethyl-[1,3,2]dioxaborolan-2-yl)-dibenzofuran-1-yl]-[1,3,5]triazine

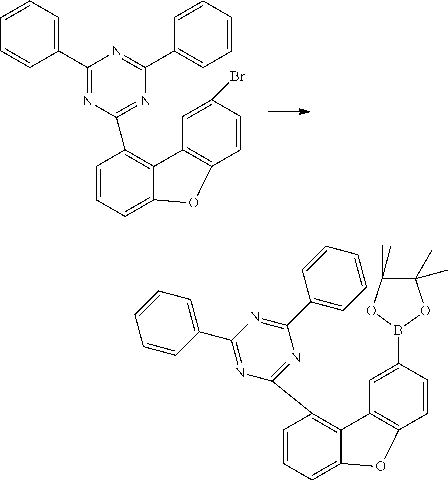 [SMILES: Brc1ccc2oc3cccc(-c4nc(-c5ccccc5)nc(-c5ccccc5)n4)c3c2c1 ; CC1(C)OB(c2ccc3oc4cccc(-c5nc(-c6ccccc6)nc(-c6ccccc6)n5)c4c3c2)OC1(C)C]

In a 500 ml flask, under protective gas, 60 g (125 mmol) of 2-(8-bromodibenzofuran-1-yl)-4,6-diphenyl-[1,3,5]triazine together with 39 g (1051 mmol) of bis(pinacolato)diborane (CAS 73183-34-3) are dissolved in 900 ml of dry DMF and the mixture is degassed for 30 minutes. Subsequently, 37 g (376 mmol) of potassium acetate and 1.9 g (8.7 mmol) of palladium acetate are added, and the mixture is heated to 80° C. overnight. After the reaction has ended, the mixture is diluted with 300 ml of toluene and extracted with water. The solvent is removed on a rotary evaporator and recrystallized with heptane. Yield: 61 g (117 mmol), 94% of theory.

The following compounds are prepared in an analogous manner:

| | Reactant 1 | Product | Yield |
|---|---|---|---|
| k1 | 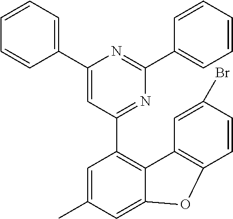 [SMILES: Cc1cc(-c2cc(-c3ccccc3)nc(-c3ccccc3)n2)c2c(c1)oc1ccc(Br)cc12] | 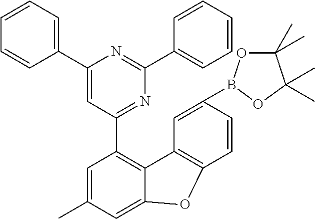 [SMILES: Cc1cc(-c2cc(-c3ccccc3)nc(-c3ccccc3)n2)c2c(c1)oc1ccc(B3OC(C)(C)C(C)(C)O3)cc12] | 80% |
| k2 | 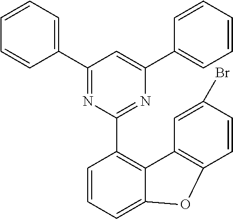 [SMILES: Brc1ccc2oc3cccc(-c4nc(-c5ccccc5)cc(-c5ccccc5)n4)c3c2c1] | 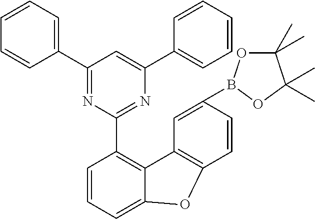 [SMILES: CC1(C)OB(c2ccc3oc4cccc(-c5nc(-c6ccccc6)cc(-c6ccccc6)n5)c4c3c2)OC1(C)C] | 83% |
| k3 | 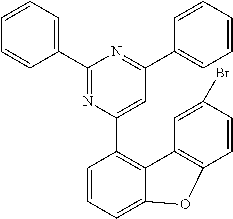 [SMILES: Brc1ccc2oc3cccc(-c4cc(-c5ccccc5)nc(-c5ccccc5)n4)c3c2c1] | 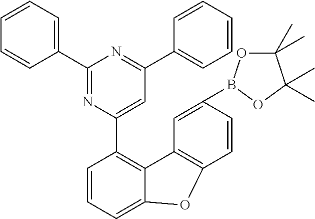 [SMILES: CC1(C)OB(c2ccc3oc4cccc(-c5cc(-c6ccccc6)nc(-c6ccccc6)n5)c4c3c2)OC1(C)C] | 85% |
| k4 | 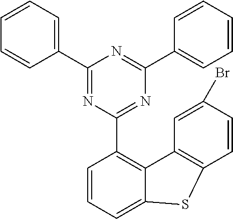 [SMILES: Brc1ccc2sc3cccc(-c4nc(-c5ccccc5)nc(-c5ccccc5)n4)c3c2c1] | 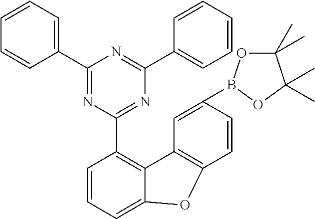 [SMILES: CC1(C)OB(c2ccc3oc4cccc(-c5nc(-c6ccccc6)nc(-c6ccccc6)n5)c4c3c2)OC1(C)C] | 87% |
| k5 | 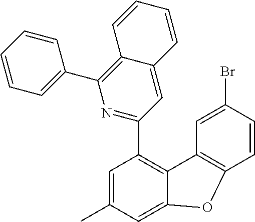 [SMILES: Cc1cc(-c2cc3ccccc3c(-c3ccccc3)n2)c2c(c1)oc1ccc(Br)cc12] | 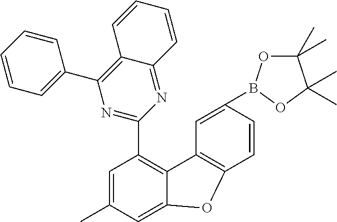 [SMILES: Cc1cc(-c2nc(-c3ccccc3)c3ccccc3n2)c2c(c1)oc1ccc(B3OC(C)(C)C(C)(C)O3)cc12] | 83% |
| k6 | 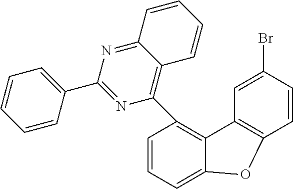 [SMILES: Brc1ccc2oc3cccc(-c4nc(-c5ccccc5)nc5ccccc45)c3c2c1] | 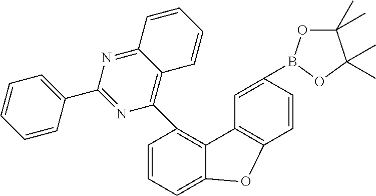 [SMILES: CC1(C)OB(c2ccc3oc4cccc(-c5nc(-c6ccccc6)nc6ccccc56)c4c3c2)OC1(C)C] | 81% |
| k7 | 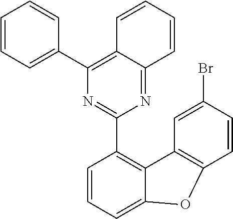 [SMILES: Brc1ccc2oc3cccc(-c4nc(-c5ccccc5)c5ccccc5n4)c3c2c1] | 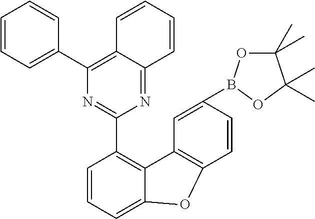 [SMILES: CC1(C)OB(c2ccc3oc4cccc(-c5nc(-c6ccccc6)c6ccccc6n5)c4c3c2)OC1(C)C] | 92% |
| k8 | 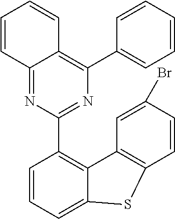 [SMILES: Brc1ccc2sc3cccc(-c4nc(-c5ccccc5)c5ccccc5n4)c3c2c1] | 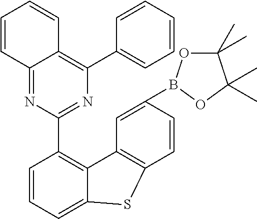 [SMILES: CC1(C)OB(c2ccc3sc4cccc(-c5nc(-c6ccccc6)c6ccccc6n5)c4c3c2)OC1(C)C] | 90% |
| k9 | 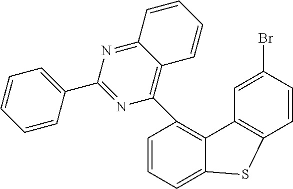 [SMILES: Brc1ccc2sc3cccc(-c4nc(-c5ccccc5)nc5ccccc45)c3c2c1] | 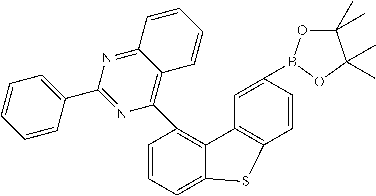 [SMILES: CC1(C)OB(c2ccc3sc4cccc(-c5nc(-c6ccccc6)nc6ccccc56)c4c3c2)OC1(C)C] | 87% |
| k10 | 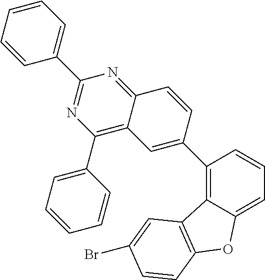 [SMILES: Brc1ccc2oc3cccc(-c4ccc5nc(-c6ccccc6)nc(-c6ccccc6)c5c4)c3c2c1] | 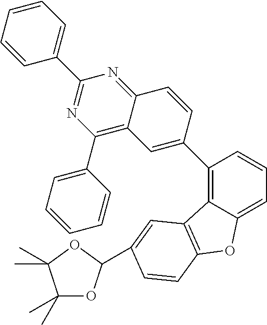 [SMILES: CC1(C)OC(c2ccc3oc4cccc(-c5ccc6nc(-c7ccccc7)nc(-c7ccccc7)c6c5)c4c3c2)OC1(C)C] | 89% |

### l) 2,4-Diphenyl-6-[8-(2,4-Diphenyl-[1,3,5]triazin-2-yl)dibenzofuran-1-yl]-[1,3,5]triazine

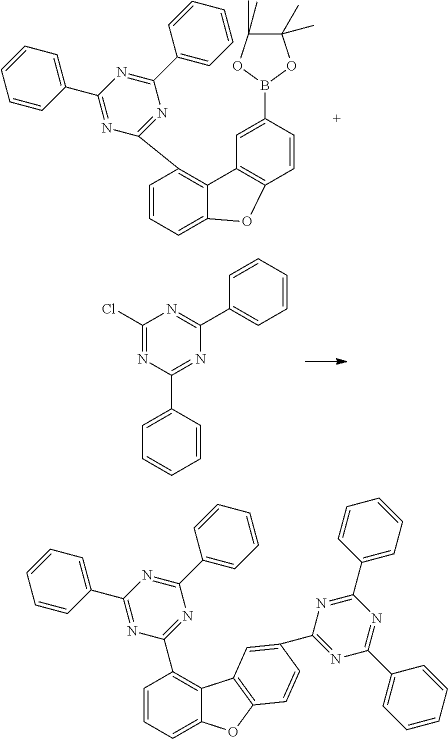

68.7 g (110.0 mmol) of 2,4-diphenyl-6-[8-(4,4,5,5-tetramethyl-[1,3,2]dioxaborolan-2-yl)-dibenzofuran-1-yl]-[1,3,5]triazine, 29.3 g (110.0 mmol) of 2-chloro-4,6-diphenyl-1,3,5-triazine and 21 g (210.0 mmol) of sodium carbonate are suspended in 500 ml of ethylene glycol diamine ether and 500 ml of water. Added to this suspension are 913 mg (3.0 mmol) of tri-o-tolylphosphine and then 112 mg (0.5 mmol) of palladium(II) acetate, and the reaction mixture is heated under reflux for 16 h. After cooling, the organic phase is removed, filtered through silica gel, washed three times with 200 ml of water and then concentrated to dryness. The product is purified via column chromatography on silica gel with toluene/CHCl3 (1:1) and finally sublimed under high vacuum (p=5×10⁻⁷ mbar) (99.9% purity). The yield is 51 g (81 mmol), corresponding to 74% of theory.

The following compounds are prepared in an analogous manner:

| | Reactant 1 | Reactant 2 |
|---|---|---|
| l1 | 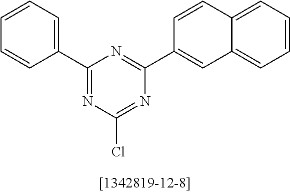 [SMILES: Clc1nc(-c2ccccc2)nc(-c2ccc3ccccc3c2)n1] | 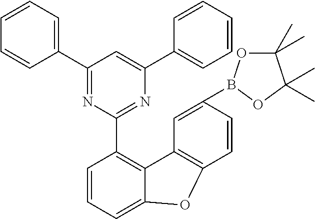 [SMILES: CC1(C)OB(c2ccc3oc4cccc(-c5nc(-c6ccccc6)cc(-c6ccccc6)n5)c4c3c2)OC1(C)C] |
| l2 | 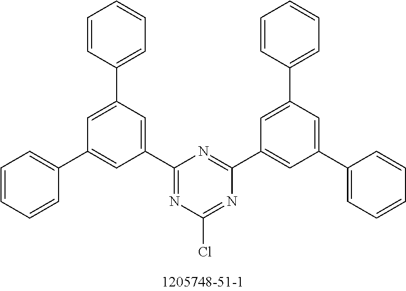 [SMILES: Clc1nc(-c2cc(-c3ccccc3)cc(-c3ccccc3)c2)nc(-c2cc(-c3ccccc3)cc(-c3ccccc3)c2)n1] | 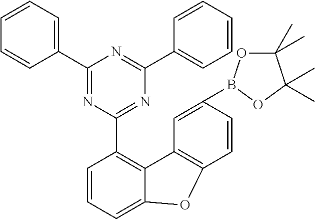 [SMILES: CC1(C)OB(c2ccc3oc4cccc(-c5nc(-c6ccccc6)nc(-c6ccccc6)n5)c4c3c2)OC1(C)C] |
| l3 | 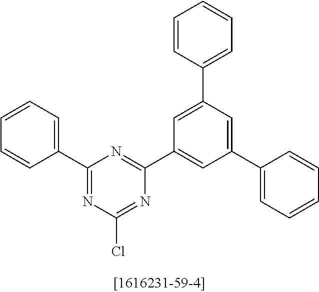 [SMILES: Clc1nc(-c2ccccc2)nc(-c2cc(-c3ccccc3)cc(-c3ccccc3)c2)n1] | 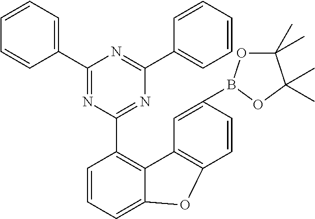 [SMILES: CC1(C)OB(c2ccc3oc4cccc(-c5nc(-c6ccccc6)nc(-c6ccccc6)n5)c4c3c2)OC1(C)C] |
| l4 | 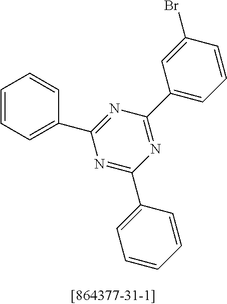 [SMILES: Brc1cccc(-c2nc(-c3ccccc3)nc(-c3ccccc3)n2)c1] | 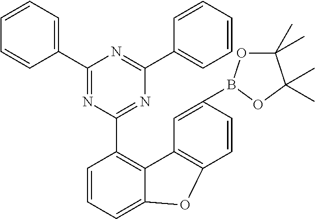 [SMILES: CC1(C)OB(c2ccc3oc4cccc(-c5nc(-c6ccccc6)nc(-c6ccccc6)n5)c4c3c2)OC1(C)C] |
| l5 | 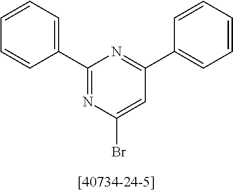 [SMILES: Brc1cc(-c2ccccc2)nc(-c2ccccc2)n1] | 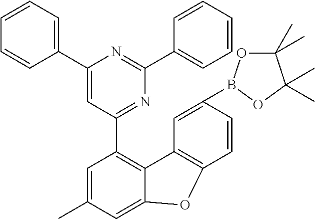 [SMILES: Cc1cc(-c2cc(-c3ccccc3)nc(-c3ccccc3)n2)c2c(c1)oc1ccc(B3OC(C)(C)C(C)(C)O3)cc12] |
| l6 | 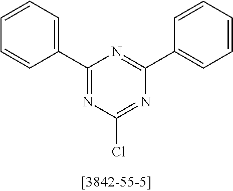 [SMILES: Clc1nc(-c2ccccc2)nc(-c2ccccc2)n1] | 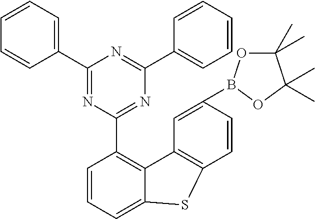 [SMILES: CC1(C)OB(c2ccc3sc4cccc(-c5nc(-c6ccccc6)nc(-c6ccccc6)n5)c4c3c2)OC1(C)C] |
| l7 | 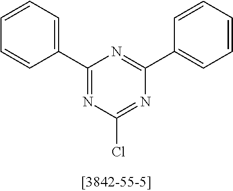 [SMILES: Clc1nc(-c2ccccc2)nc(-c2ccccc2)n1] | 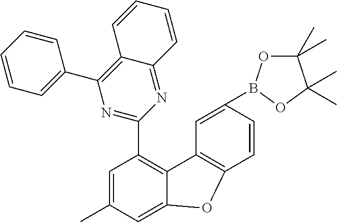 [SMILES: Cc1cc(-c2nc(-c3ccccc3)c3ccccc3n2)c2c(c1)oc1ccc(B3OC(C)(C)C(C)(C)O3)cc12] |
| l8 | 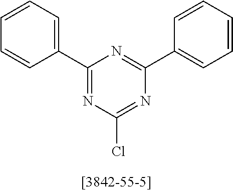 [SMILES: Clc1nc(-c2ccccc2)nc(-c2ccccc2)n1] | 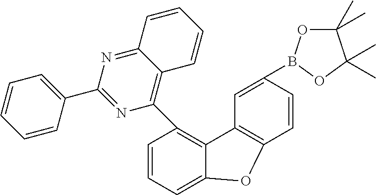 [SMILES: CC1(C)OB(c2ccc3oc4cccc(-c5nc(-c6ccccc6)nc6ccccc56)c4c3c2)OC1(C)C] |
| l9 | 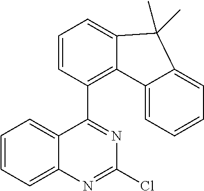 [SMILES: CC1(C)c2ccccc2-c2c(-c3nc(Cl)nc4ccccc34)cccc21] | 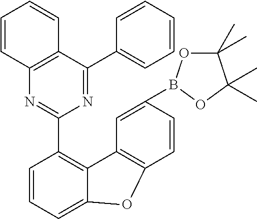 [SMILES: CC1(C)OB(c2ccc3oc4cccc(-c5nc(-c6ccccc6)c6ccccc6n5)c4c3c2)OC1(C)C] |
| l19 | 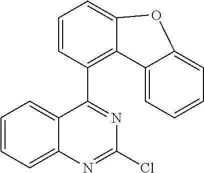 [SMILES: Clc1nc(-c2cccc3oc4ccccc4c23)c2ccccc2n1] | 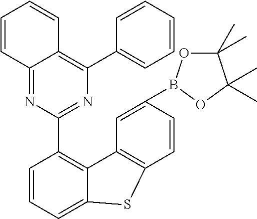 [SMILES: CC1(C)OB(c2ccc3sc4cccc(-c5nc(-c6ccccc6)c6ccccc6n5)c4c3c2)OC1(C)C] |
| l20 | 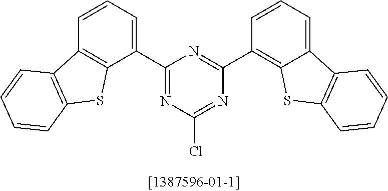 [SMILES: Clc1nc(-c2cccc3c2sc2ccccc23)nc(-c2cccc3c2sc2ccccc23)n1] | 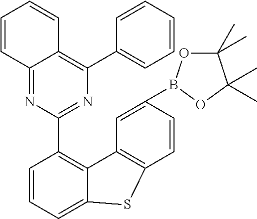 [SMILES: CC1(C)OB(c2ccc3sc4cccc(-c5nc(-c6ccccc6)c6ccccc6n5)c4c3c2)OC1(C)C] |
| l21 | 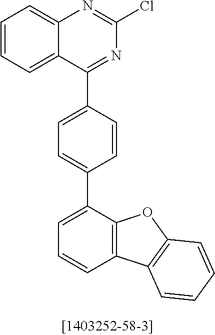 [SMILES: Clc1nc(-c2ccc(-c3cccc4c3oc3ccccc34)cc2)c2ccccc2n1] | 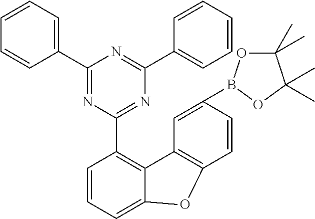 [SMILES: CC1(C)OB(c2ccc3oc4cccc(-c5nc(-c6ccccc6)nc(-c6ccccc6)n5)c4c3c2)OC1(C)C] |
| l22 | 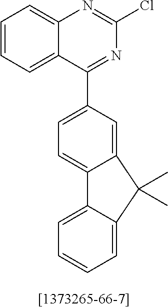 [SMILES: CC1(C)c2ccccc2-c2ccc(-c3nc(Cl)nc4ccccc34)cc21] | 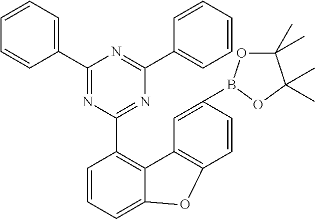 [SMILES: CC1(C)OB(c2ccc3oc4cccc(-c5nc(-c6ccccc6)nc(-c6ccccc6)n5)c4c3c2)OC1(C)C] |
| l23 | 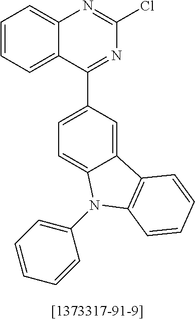 [SMILES: Clc1nc(-c2ccc3c(c2)c2ccccc2n3-c2ccccc2)c2ccccc2n1] | 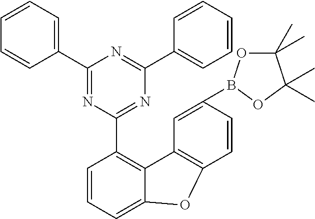 [SMILES: CC1(C)OB(c2ccc3oc4cccc(-c5nc(-c6ccccc6)nc(-c6ccccc6)n5)c4c3c2)OC1(C)C] |
| l24 | 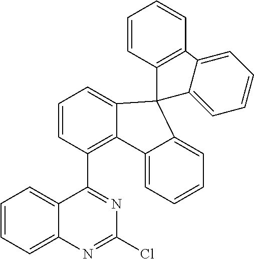 [SMILES: Clc1nc(-c2cccc3c2-c2ccccc2C32c3ccccc3-c3ccccc32)c2ccccc2n1] | 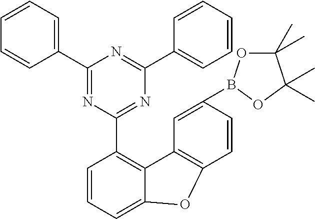 [SMILES: CC1(C)OB(c2ccc3oc4cccc(-c5nc(-c6ccccc6)nc(-c6ccccc6)n5)c4c3c2)OC1(C)C] |
| l25 | 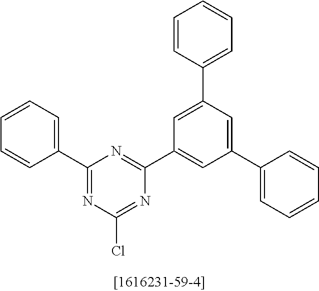 [SMILES: Clc1nc(-c2ccccc2)nc(-c2cc(-c3ccccc3)cc(-c3ccccc3)c2)n1] | 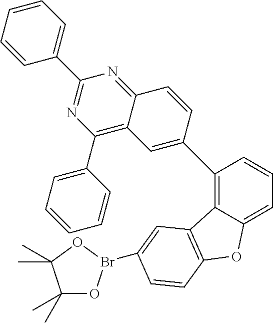 |
| l26 | 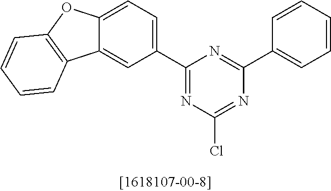 [SMILES: Clc1nc(-c2ccccc2)nc(-c2ccc3oc4ccccc4c3c2)n1] | 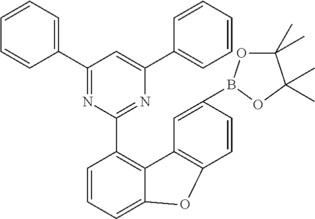 [SMILES: CC1(C)OB(c2ccc3oc4cccc(-c5nc(-c6ccccc6)cc(-c6ccccc6)n5)c4c3c2)OC1(C)C] |
| l27 | 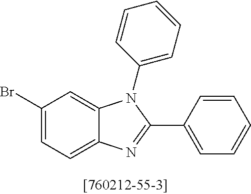 [SMILES: Brc1ccc2nc(-c3ccccc3)n(-c3ccccc3)c2c1] | 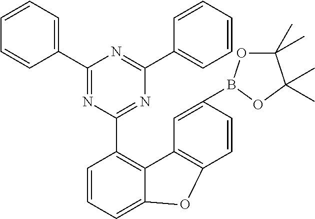 [SMILES: CC1(C)OB(c2ccc3oc4cccc(-c5nc(-c6ccccc6)nc(-c6ccccc6)n5)c4c3c2)OC1(C)C] |
| l28 | 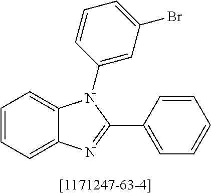 [SMILES: Brc1cccc(-n2c(-c3ccccc3)nc3ccccc32)c1] | 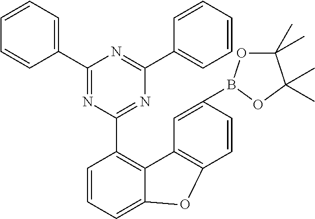 [SMILES: CC1(C)OB(c2ccc3oc4cccc(-c5nc(-c6ccccc6)nc(-c6ccccc6)n5)c4c3c2)OC1(C)C] |
| l29 | 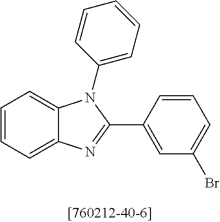 [SMILES: Brc1cccc(-c2nc3ccccc3n2-c2ccccc2)c1] | 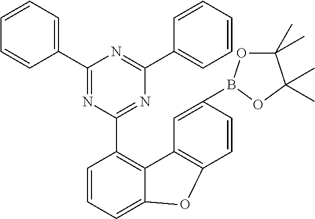 [SMILES: CC1(C)OB(c2ccc3oc4cccc(-c5nc(-c6ccccc6)nc(-c6ccccc6)n5)c4c3c2)OC1(C)C] |
| l30 | 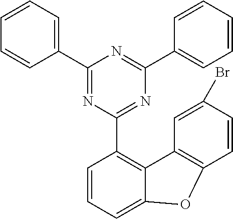 [SMILES: Brc1ccc2oc3cccc(-c4nc(-c5ccccc5)nc(-c5ccccc5)n4)c3c2c1] | 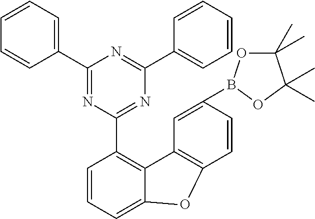 [SMILES: CC1(C)OB(c2ccc3oc4cccc(-c5nc(-c6ccccc6)nc(-c6ccccc6)n5)c4c3c2)OC1(C)C] |
| l31 | 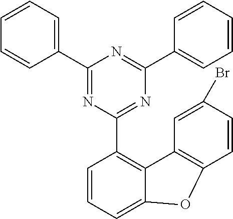 [SMILES: Brc1ccc2oc3cccc(-c4nc(-c5ccccc5)nc(-c5ccccc5)n4)c3c2c1] | 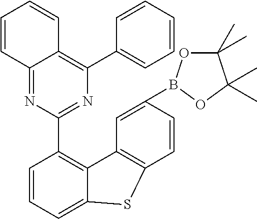 [SMILES: CC1(C)OB(c2ccc3sc4cccc(-c5nc(-c6ccccc6)c6ccccc6n5)c4c3c2)OC1(C)C] |
| l32 | 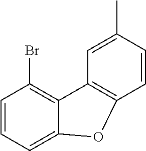 | 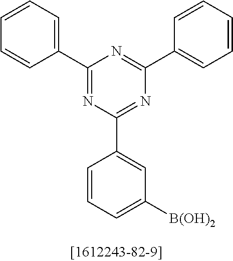 [SMILES: OB(O)c1cccc(-c2nc(-c3ccccc3)nc(-c3ccccc3)n2)c1] |
| l33 | 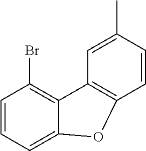 | 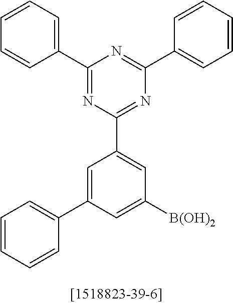 [SMILES: OB(O)c1cc(-c2ccccc2)cc(-c2nc(-c3ccccc3)nc(-c3ccccc3)n2)c1] |
| l34 | 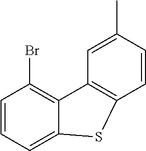 | 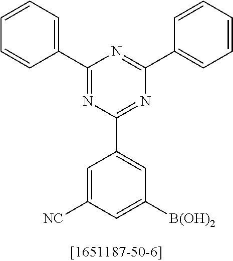 [SMILES: N#Cc1cc(B(O)O)cc(-c2nc(-c3ccccc3)nc(-c3ccccc3)n2)c1] |
| l35 | 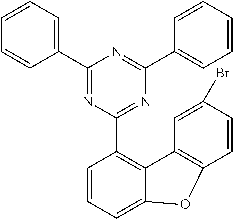 [SMILES: Brc1ccc2oc3cccc(-c4nc(-c5ccccc5)nc(-c5ccccc5)n4)c3c2c1] | 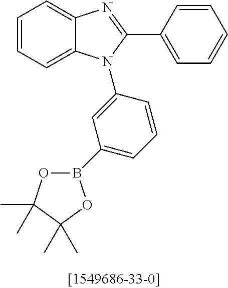 [SMILES: CC1(C)OB(c2cccc(-n3c(-c4ccccc4)nc4ccccc43)c2)OC1(C)C] |
| l36 | 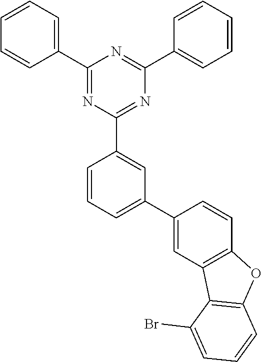 [SMILES: Brc1cccc2oc3ccc(-c4cccc(-c5nc(-c6ccccc6)nc(-c6ccccc6)n5)c4)cc3c12] | 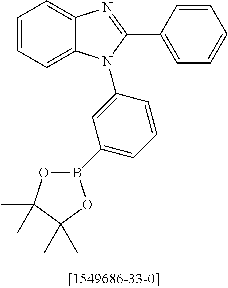 [SMILES: CC1(C)OB(c2cccc(-n3c(-c4ccccc4)nc4ccccc43)c2)OC1(C)C] |
| l37 | 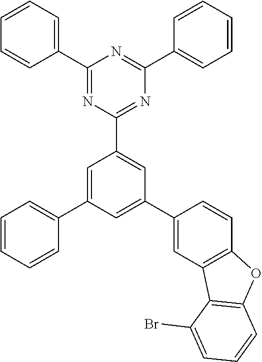 [SMILES: Brc1cccc2oc3ccc(-c4cc(-c5ccccc5)cc(-c5nc(-c6ccccc6)nc(-c6ccccc6)n5)c4)cc3c12] | 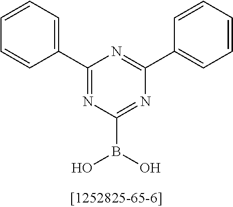 [SMILES: OB(O)c1nc(-c2ccccc2)nc(-c2ccccc2)n1] |
| l38 | 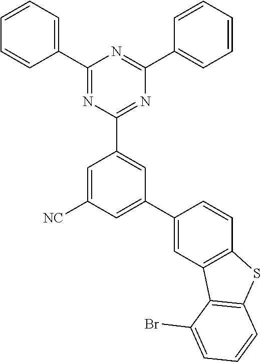 [SMILES: N#Cc1cc(-c2ccc3sc4cccc(Br)c4c3c2)cc(-c2nc(-c3ccccc3)nc(-c3ccccc3)n2)c1] | 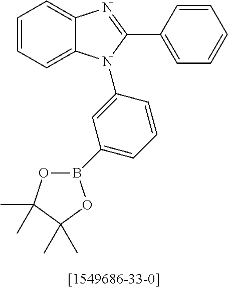 [SMILES: CC1(C)OB(c2cccc(-n3c(-c4ccccc4)nc4ccccc43)c2)OC1(C)C] |
| l39 | 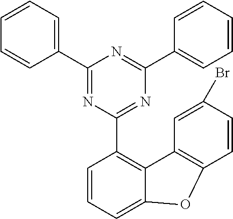 [SMILES: Brc1ccc2oc3cccc(-c4nc(-c5ccccc5)nc(-c5ccccc5)n4)c3c2c1] | 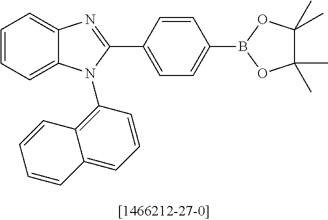 [SMILES: CC1(C)OB(c2ccc(-c3nc4ccccc4n3-c3cccc4ccccc34)cc2)OC1(C)C] |
| l40 | 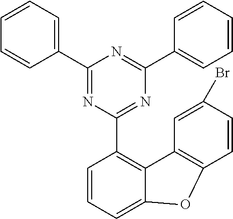 [SMILES: Brc1ccc2oc3cccc(-c4nc(-c5ccccc5)nc(-c5ccccc5)n4)c3c2c1] | 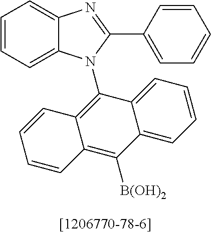 [SMILES: OB(O)c1c2ccccc2c(-n2c(-c3ccccc3)nc3ccccc32)c2ccccc12] |
| l41 | 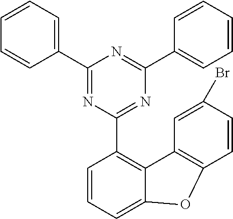 [SMILES: Brc1ccc2oc3cccc(-c4nc(-c5ccccc5)nc(-c5ccccc5)n4)c3c2c1] | 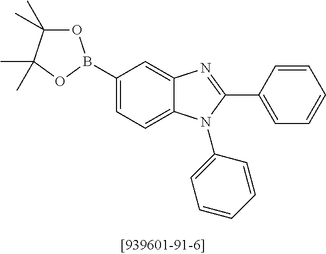 [SMILES: CC1(C)OB(c2ccc3c(c2)nc(-c2ccccc2)n3-c2ccccc2)OC1(C)C] |

| | Product | Yield |
|---|---|---|
| l1 | 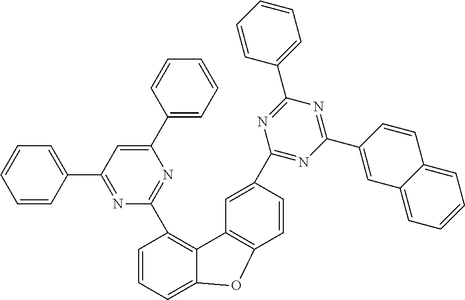 [SMILES: c1ccc(-c2cc(-c3ccccc3)nc(-c3cccc4oc5ccc(-c6nc(-c7ccccc7)nc(-c7ccc8ccccc8c7)n6)cc5c34)n2)cc1] | 61% |
| l2 | 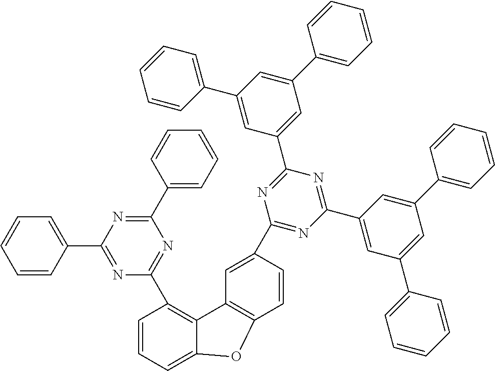 [SMILES: c1ccc(-c2cc(-c3ccccc3)cc(-c3nc(-c4cc(-c5ccccc5)cc(-c5ccccc5)c4)nc(-c4ccc5oc6cccc(-c7nc(-c8ccccc8)nc(-c8ccccc8)n7)c6c5c4)n3)c2)cc1] | 65% |
| l3 | 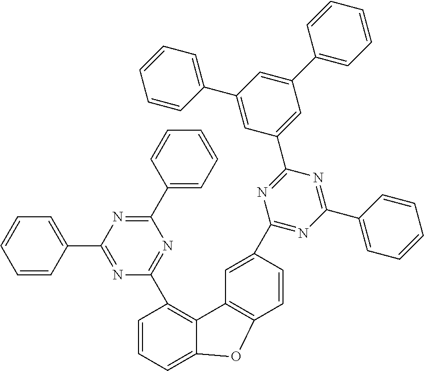 [SMILES: c1ccc(-c2cc(-c3ccccc3)cc(-c3nc(-c4ccccc4)nc(-c4ccc5oc6cccc(-c7nc(-c8ccccc8)nc(-c8ccccc8)n7)c6c5c4)n3)c2)cc1] | 67% |
| l4 | 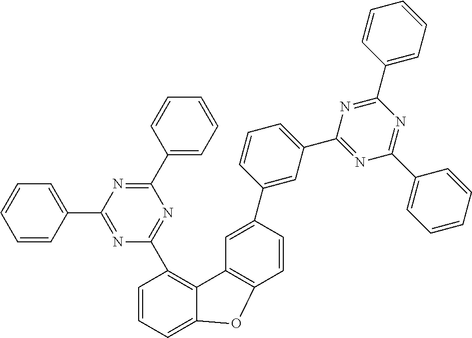 [SMILES: c1ccc(-c2nc(-c3ccccc3)nc(-c3cccc(-c4ccc5oc6cccc(-c7nc(-c8ccccc8)nc(-c8ccccc8)n7)c6c5c4)c3)n2)cc1] | 70% |
| l5 | 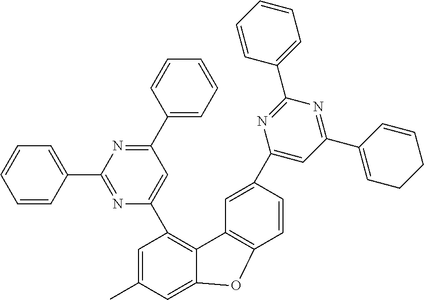 [SMILES: Cc1cc(-c2cc(-c3ccccc3)nc(-c3ccccc3)n2)c2c(c1)oc1ccc(-c3cc(C4=CCCC=C4)nc(-c4ccccc4)n3)cc12] | 65% |
| l6 | 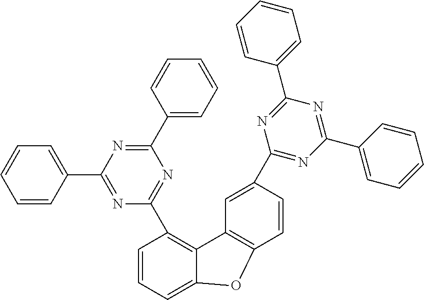 [SMILES: c1ccc(-c2nc(-c3ccccc3)nc(-c3ccc4oc5cccc(-c6nc(-c7ccccc7)nc(-c7ccccc7)n6)c5c4c3)n2)cc1] | 72% |
| l7 | 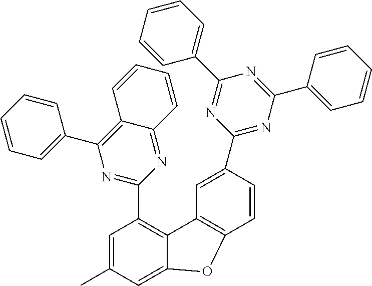 [SMILES: Cc1cc(-c2nc(-c3ccccc3)c3ccccc3n2)c2c(c1)oc1ccc(-c3nc(-c4ccccc4)nc(-c4ccccc4)n3)cc12] | 79% |
| l8 | 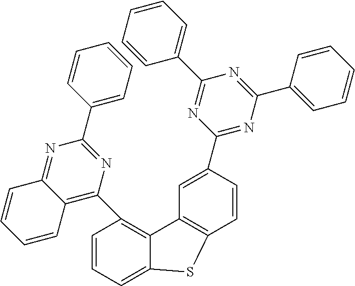 [SMILES: c1ccc(-c2nc(-c3ccccc3)nc(-c3ccc4sc5cccc(-c6nc(-c7ccccc7)nc7ccccc67)c5c4c3)n2)cc1] | 76% |
| l9 | 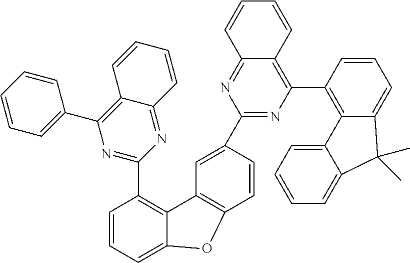 [SMILES: CC1(C)c2ccccc2-c2c(-c3nc(-c4ccc5oc6cccc(-c7nc(-c8ccccc8)c8ccccc8n7)c6c5c4)nc4ccccc34)cccc21] | 78% |
| l19 | 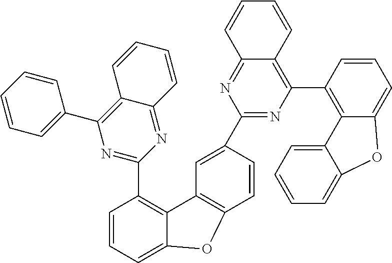 [SMILES: c1ccc(-c2nc(-c3cccc4oc5ccc(-c6nc(-c7cccc8oc9ccccc9c78)c7ccccc7n6)cc5c34)nc3ccccc23)cc1] | 73% |
| l20 | 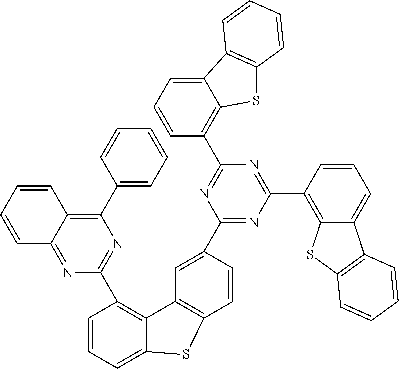 [SMILES: c1ccc(-c2nc(-c3cccc4sc5ccc(-c6nc(-c7cccc8c7sc7ccccc78)nc(-c7cccc8c7sc7ccccc78)n6)cc5c34)nc3ccccc23)cc1] | 76% |
| l21 | 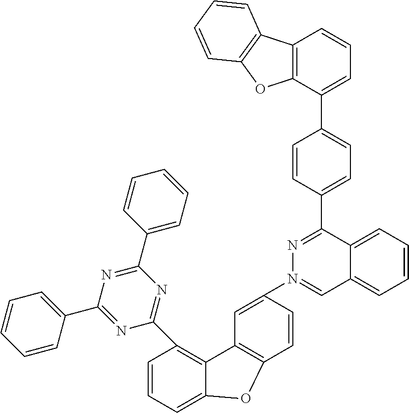 | 73% |
| l22 | 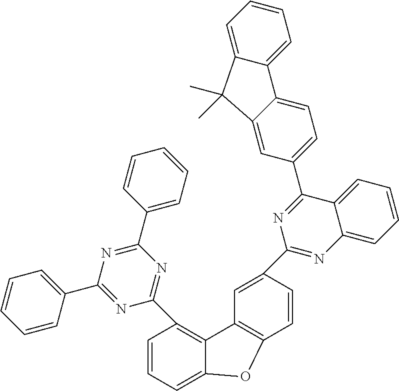 [SMILES: CC1(C)c2ccccc2-c2ccc(-c3nc(-c4ccc5oc6cccc(-c7nc(-c8ccccc8)nc(-c8ccccc8)n7)c6c5c4)nc4ccccc34)cc21] | 71% |
| l23 | 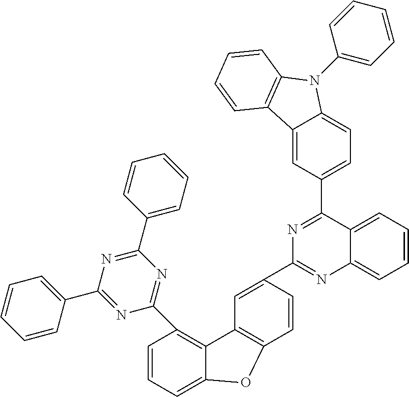 [SMILES: c1ccc(-c2nc(-c3ccccc3)nc(-c3cccc4oc5ccc(-c6nc(-c7ccc8c(c7)c7ccccc7n8-c7ccccc7)c7ccccc7n6)cc5c34)n2)cc1] | 76% |
| l24 | 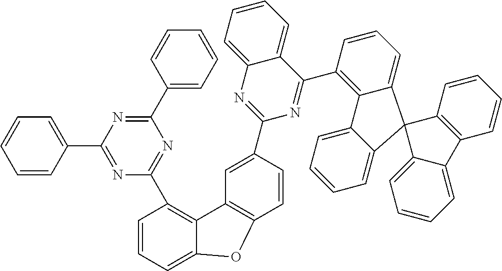 [SMILES: c1ccc(-c2nc(-c3ccccc3)nc(-c3cccc4oc5ccc(-c6nc(-c7cccc8c7-c7ccccc7C87c8ccccc8-c8ccccc87)c7ccccc7n6)cc5c34)n2)cc1] | 59% |
| l25 | 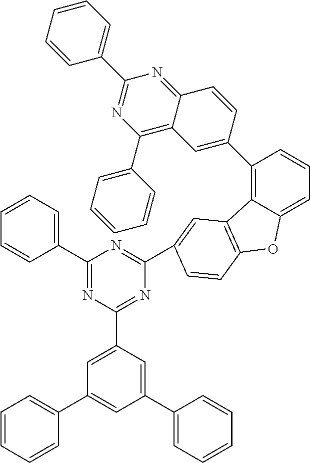 [SMILES: c1ccc(-c2cc(-c3ccccc3)cc(-c3nc(-c4ccccc4)nc(-c4ccc5oc6cccc(-c7ccc8nc(-c9ccccc9)nc(-c9ccccc9)c8c7)c6c5c4)n3)c2)cc1] | 78% |
| l26 | 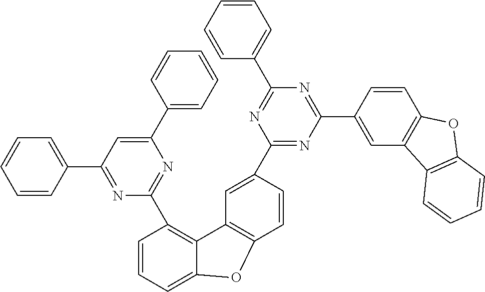 [SMILES: c1ccc(-c2cc(-c3ccccc3)nc(-c3cccc4oc5ccc(-c6nc(-c7ccccc7)nc(-c7ccc8oc9ccccc9c8c7)n6)cc5c34)n2)cc1] | 71% |
| l27 | 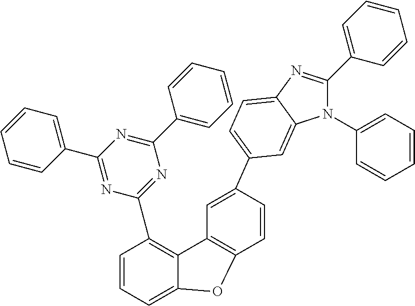 [SMILES: c1ccc(-c2nc(-c3ccccc3)nc(-c3cccc4oc5ccc(-c6ccc7nc(-c8ccccc8)n(-c8ccccc8)c7c6)cc5c34)n2)cc1] | 72% |
| l28 | 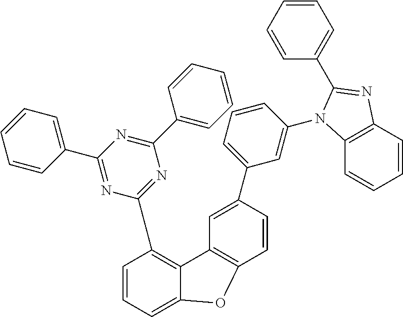 [SMILES: c1ccc(-c2nc(-c3ccccc3)nc(-c3cccc4oc5ccc(-c6cccc(-n7c(-c8ccccc8)nc8ccccc87)c6)cc5c34)n2)cc1] | 78% |
| l29 | 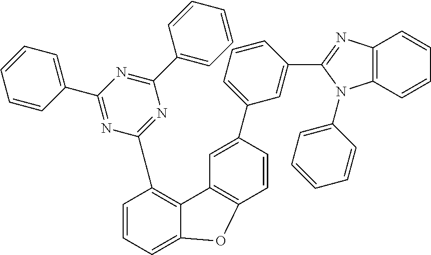 [SMILES: c1ccc(-c2nc(-c3ccccc3)nc(-c3cccc4oc5ccc(-c6cccc(-c7nc8ccccc8n7-c7ccccc7)c6)cc5c34)n2)cc1] | 73% |
| l30 | 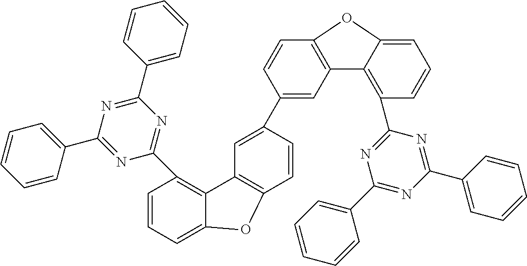 [SMILES: c1ccc(-c2nc(-c3ccccc3)nc(-c3cccc4oc5ccc(-c6ccc7oc8cccc(-c9nc(-c%10ccccc%10)nc(-c%10ccccc%10)n9)c8c7c6)cc5c34)n2)cc1] | 65% |
| l31 | 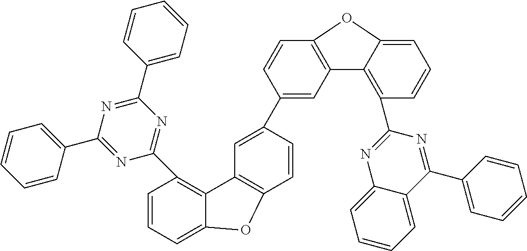 [SMILES: c1ccc(-c2nc(-c3ccccc3)nc(-c3cccc4oc5ccc(-c6ccc7oc8cccc(-c9nc(-c%10ccccc%10)c%10ccccc%10n9)c8c7c6)cc5c34)n2)cc1] | 63% |
| l32 | 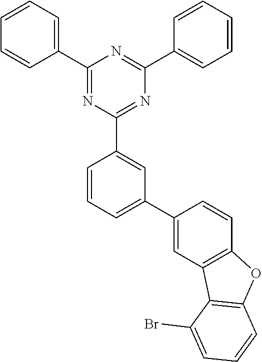 [SMILES: Brc1cccc2oc3ccc(-c4cccc(-c5nc(-c6ccccc6)nc(-c6ccccc6)n5)c4)cc3c12] | 62% |
| l33 | 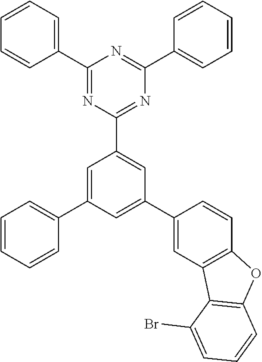 [SMILES: Brc1cccc2oc3ccc(-c4cc(-c5ccccc5)cc(-c5nc(-c6ccccc6)nc(-c6ccccc6)n5)c4)cc3c12] | 57% |
| l34 | 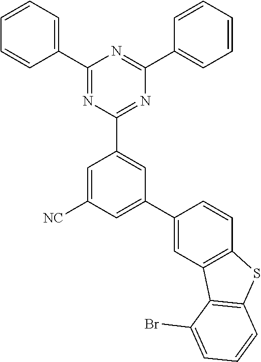 [SMILES: N#Cc1cc(-c2ccc3sc4cccc(Br)c4c3c2)cc(-c2nc(-c3ccccc3)nc(-c3ccccc3)n2)c1] | 64% |
| l35 | 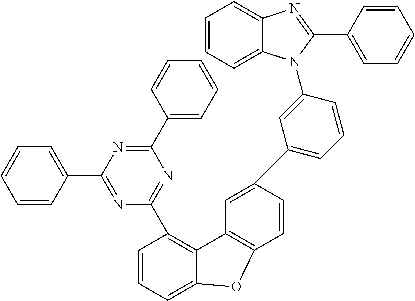 [SMILES: c1ccc(-c2nc(-c3ccccc3)nc(-c3cccc4oc5ccc(-c6cccc(-n7c(-c8ccccc8)nc8ccccc87)c6)cc5c34)n2)cc1] | 63% |
| l36 | 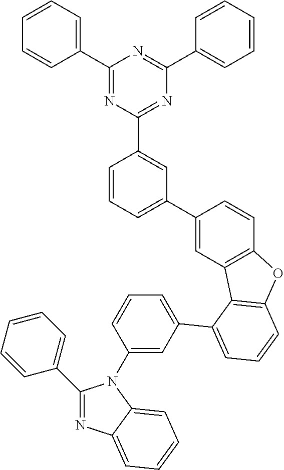 [SMILES: c1ccc(-c2nc(-c3ccccc3)nc(-c3cccc(-c4ccc5oc6cccc(-c7cccc(-n8c(-c9ccccc9)nc9ccccc98)c7)c6c5c4)c3)n2)cc1] | 60% |
| l37 | 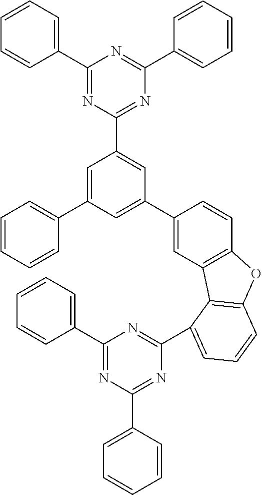 [SMILES: c1ccc(-c2cc(-c3ccc4oc5cccc(-c6nc(-c7ccccc7)nc(-c7ccccc7)n6)c5c4c3)cc(-c3nc(-c4ccccc4)nc(-c4ccccc4)n3)c2)cc1] | 62% |
| l38 | 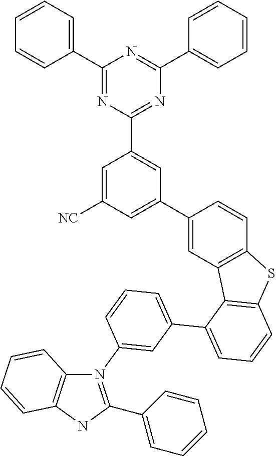 | 65% |
| l39 | 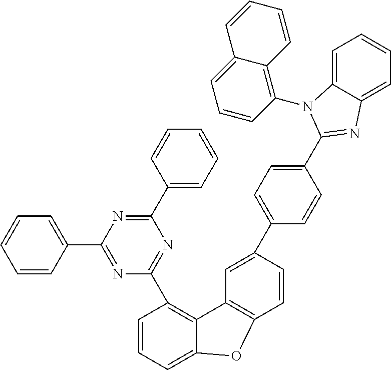 [SMILES: c1ccc(-c2nc(-c3ccccc3)nc(-c3cccc4oc5ccc(-c6ccc(-c7nc8ccccc8n7-c7cccc8ccccc78)cc6)cc5c34)n2)cc1] | 71% |
| l40 | 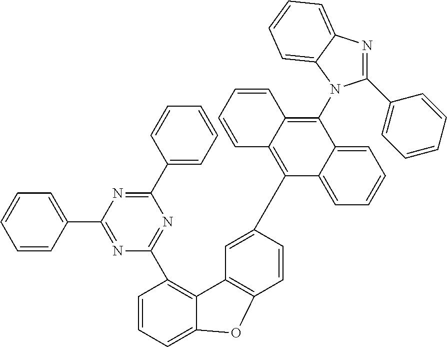 [SMILES: c1ccc(-c2nc(-c3ccccc3)nc(-c3cccc4oc5ccc(-c6c7ccccc7c(-n7c(-c8ccccc8)nc8ccccc87)c7ccccc67)cc5c34)n2)cc1] | 64% |
| l41 | 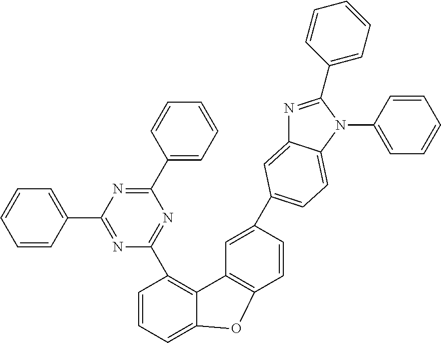 [SMILES: c1ccc(-c2nc(-c3ccccc3)nc(-c3cccc4oc5ccc(-c6ccc7c(c6)nc(-c6ccccc6)n7-c6ccccc6)cc5c34)n2)cc1] | 61% |

### m) 1-[9-(4,6-Diphenyl-[1,3,5]triazin-2-yl)-dibenzofuran-2-yl]-1H-benzoimidazole

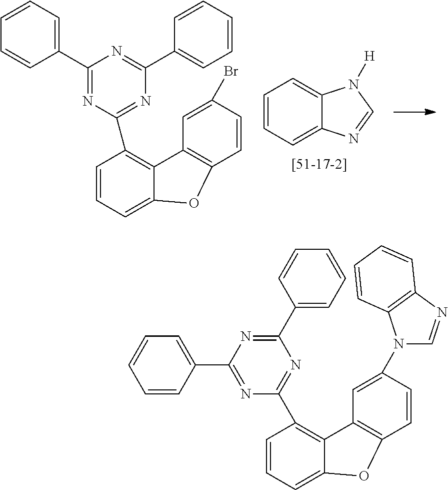

Under protective gas, 10 g (84.7 mmol) of benzimidazole, 42 g (127.4 mmol) of CsCO₃, 2.4 g (14.7 mmol) of CuI and 30 g (63 mmol) of 2-(8-bromodibenzofuran-1-yl)-4,6-diphenyl-[1,3,5]triazine are suspended in 100 ml of degassed DMF, and the reaction mixture is heated under reflux at 120° C. for 40 h. After cooling, the solvent is removed under reduced pressure, the residue is dissolved in dichloromethane and water is added. Thereafter, the organic phase is removed and filtered through silica gel. The yield is 27.9 g (54 mmol), corresponding to 86% of theory.

The following compounds are prepared in an analogous manner:

| | Reactant 1 | Reactant 2 | Product | Yield |
|---|---|---|---|---|
| m1 | 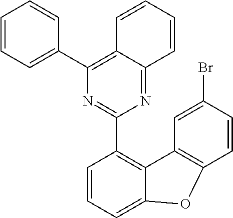 [SMILES: Brc1ccc2oc3cccc(-c4nc(-c5ccccc5)c5ccccc5n4)c3c2c1] | 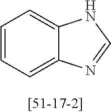 | 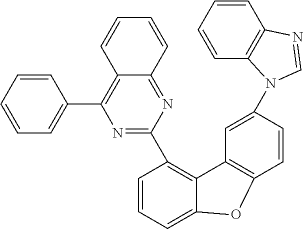 [SMILES: c1ccc(-c2nc(-c3cccc4oc5ccc(-n6cnc7ccccc76)cc5c34)nc3ccccc23)cc1] | 78% |
| m2 | 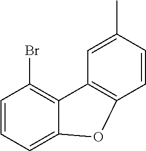 | 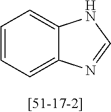 | 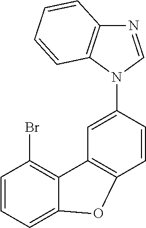 [SMILES: Brc1cccc2oc3ccc(-n4cnc5ccccc54)cc3c12] | 70% |
| m3 | 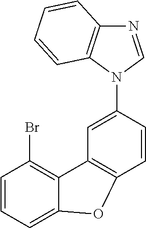 [SMILES: Brc1cccc2oc3ccc(-n4cnc5ccccc54)cc3c12] | 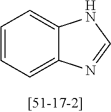 | 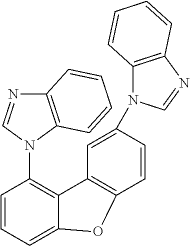 [SMILES: c1ccc2c(c1)ncn2-c1ccc2oc3cccc(-n4cnc5ccccc54)c3c2c1] | 64% |

### n) 1-[9-(4,6-Diphenyl-[1,3,5]triazin-2-yl)-dibenzofuran-2-yl]-3-phenyl-1H-benzoimidazole

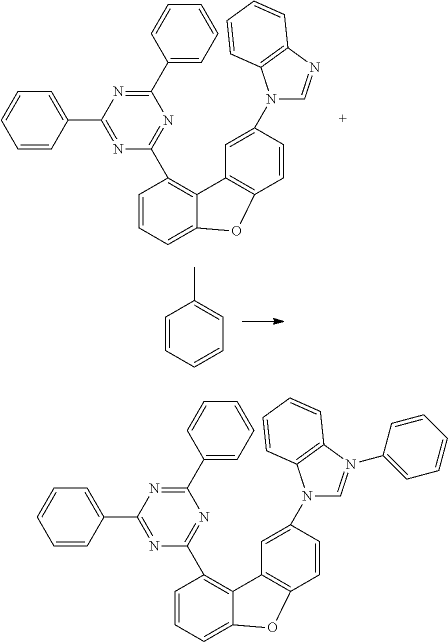

Under protective gas, 25.7 g (50 mmol) of 1-[9-(4,6-diphenyl-[1,3,5]triazin-2-yl)-dibenzofuran-2-yl]-1H-benzimidazole, 560 mg (25 mmol) of Pd(OAc)₂, 19.3 g (118 mmol) of CuI and 20.8 g (100 mmol) of iodobenzene are suspended in 300 ml of degassed DMF, and the reaction mixture is heated under reflux at 140° C. for 24 h. After cooling, the solvent is removed under reduced pressure, the residue is dissolved in dichloromethane and water is added. Thereafter, the organic phase is removed and filtered through silica gel. The product is purified via column chromatography on silica gel with toluene/heptane (1:2) and finally sublimed under high vacuum (p=5×10⁻⁷ mbar) (99.9% purity). The yield is 18.6 g (31 mmol), corresponding to 63% of theory.

The following compounds are prepared in an analogous manner:

| | Reactant 1 | Reactant 2 |
|---|---|---|
| n1 | 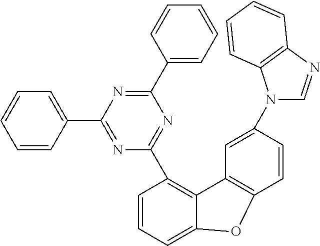 [SMILES: c1ccc(-c2nc(-c3ccccc3)nc(-c3cccc4oc5ccc(-n6cnc7ccccc76)cc5c34)n2)cc1] | 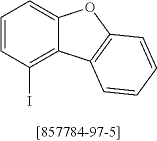 [SMILES: Ic1cccc2oc3ccccc3c12] |
| n2 | 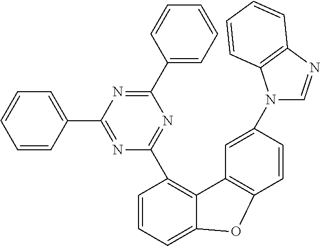 [SMILES: c1ccc(-c2nc(-c3ccccc3)nc(-c3cccc4oc5ccc(-n6cnc7ccccc76)cc5c34)n2)cc1] | 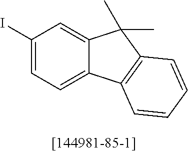 [SMILES: CC1(C)c2ccccc2-c2ccc(I)cc21] |
| n3 | 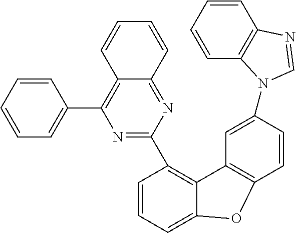 [SMILES: c1ccc(-c2nc(-c3cccc4oc5ccc(-n6cnc7ccccc76)cc5c34)nc3ccccc23)cc1] | 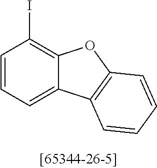 [SMILES: Ic1cccc2c1oc1ccccc12] |
| n4 | 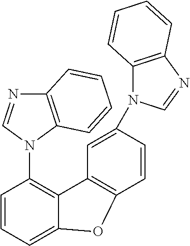 [SMILES: c1ccc2c(c1)ncn2-c1ccc2oc3cccc(-n4cnc5ccccc54)c3c2c1] | 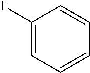 |

| | Product | Yield |
|---|---|---|
| n1 | 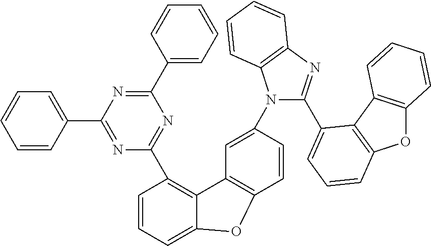 [SMILES: c1ccc(-c2nc(-c3ccccc3)nc(-c3cccc4oc5ccc(-n6c(-c7cccc8oc9ccccc9c78)nc7ccccc76)cc5c34)n2)cc1] | 45% |
| n2 | 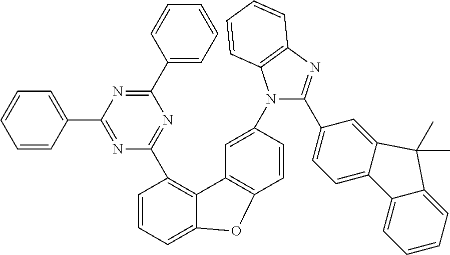 [SMILES: CC1(C)c2ccccc2-c2ccc(-c3nc4ccccc4n3-c3ccc4oc5cccc(-c6nc(-c7ccccc7)nc(-c7ccccc7)n6)c5c4c3)cc21] | 52% |
| n3 | 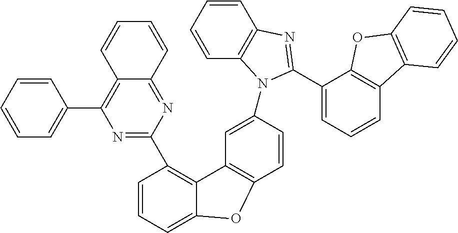 [SMILES: c1ccc(-c2nc(-c3cccc4oc5ccc(-n6c(-c7cccc8c7oc7ccccc78)nc7ccccc76)cc5c34)nc3ccccc23)cc1] | 44% |
| n4 | 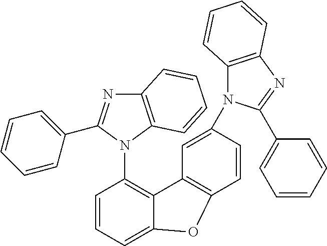 [SMILES: c1ccc(-c2nc3ccccc3n2-c2ccc3oc4cccc(-n5c(-c6ccccc6)nc6ccccc65)c4c3c2)cc1] | 41% |

Production of the OLEDs

In examples C1 to I19 which follow (see tables 1 and 2), the data of various OLEDs are presented.

Cleaned glass plaques (cleaning in laboratory glass washer, Merck Extran detergent) coated with structured ITO (indium tin oxide) of thickness 50 nm, for improved processing, are coated with 20 nm of PEDOT:PSS (poly(3,4-ethylenedioxythiophene) poly(styrenesulphonate), purchased as CLEVIOS™ P VP Al 4083 from Heraeus Precious Metals GmbH Deutschland, spun on from aqueous solution). These coated glass plaques form the substrates to which the OLEDs are applied.

The OLEDs basically have the following layer structure: substrate/hole transport layer (HTL)/interlayer (IL)/electron blocker layer (EBL)/emission layer (EML)/optional hole blocker layer (HBL)/electron transport layer (ETL)/optional electron injection layer (EIL) and finally a cathode. The cathode is formed by an aluminium layer of thickness 100 nm. The exact structure of the OLEDs can be found in Table 1. The materials required for production of the OLEDs are shown in Table 3.

All materials are applied by thermal vapour deposition in a vacuum chamber. In this case, the emission layer always consists of at least one matrix material (host material) and an emitting dopant (emitter) which is added to the matrix material(s) in a particular proportion by volume by co-evaporation. Details given in such a form as INV-1:IC3:TEG1 (60%:35%:5%) mean here that the material INV-1 is present in the layer in a proportion by volume of 60%, IC3 in a proportion of 35% and TEG1 in a proportion of 5%. Analogously, the electron transport layer may also consist of a mixture of two materials.

The OLEDs are characterized in a standard manner. For this purpose, the external quantum efficiency (EQE, measured in percent) is determined as a function of luminance, calculated from current-voltage-luminance characteristics (IUL characteristics) assuming Lambertian radiation characteristics. The parameter U1000 in Table 2 refers to the voltage which is required for a luminance of 1000 cd/m². Finally, EQE1000 refers to the external quantum efficiency at an operating luminance of 1000 cd/m². The lifetime LT is defined as the time after which the luminance drops from the starting luminance to a certain proportion L1 in the course of operation with constant current. A figure of L0;j0=4000 cd/m² and L1=70% in Table X2 means that the lifetime reported in the LT column corresponds to the time after which the starting luminance falls from 4000 cd/m² to 2800 cd/m². Analogously, L0;j0=20 mA/cm², L1=80% means that the luminance in the course of operation at 20 mA/cm² falls to 80% of its starting value after the time LT.

The data for the various OLEDs are collated in Table 2. Examples C₁-C₄ are comparative examples and show OLEDs containing materials according to the prior art. Examples I1-I19 show data for OLEDs comprising materials of the invention.

Some of the examples are elucidated in detail hereinafter, in order to illustrate the advantages of the compounds of the invention. However, it should be pointed out that this is merely a selection of the data shown in Table 2. As can be inferred from the table, even when the compounds of the invention that have not been specifically detailed are used, distinct improvements over the prior art are achieved, in some cases in all parameters, but in some cases only an improvement in efficiency, voltage or lifetime is observed. However, improvement in one of the parameters mentioned is already a significant advance because various applications require optimization with regard to different parameters.

### Use of Compounds of the Invention as Electron Transport Materials

The examples and comparative examples show that an inventive substitution leads to a distinct improvement in the voltage and lifetime without having to accept significant losses in other properties.

Compared to an OLED in which the material INV-7 is used in the ETL, in the case of use of the preferred materials INV-2, INV-3, INV-4 and INV-5, a slight improvement is observed in some cases in the voltage and in some cases also an improvement in the lifetime.

### Use of Compounds of the Invention as Matrix Materials in Phosphorescent OLEDs

The examples and comparative examples show that an inventive substitution leads to a distinct improvement in the voltage and lifetime without having to accept significant losses in other properties.

Compared to an OLED in which the material INV-6 is used in the EML, in the case of use of the preferred materials INV-1, INV-2, INV-3 and INV-4, a slight improvement is observed in some cases in the voltage and the EQE, but in particular a significant improvement in the lifetime.

TABLE 1
Structure of the OLEDs

| | HTL | IL | EBL | EML | HBL | ETL | EIL |
|---|---|---|---|---|---|---|---|
| Ex. | Thickness | Thickness | Thickness | thickness | Thickness | thickness | Thickness |
| C1 | SpA1 | HATCN | SpMA1 | VG-2:TEG1 | ST2 | ST2:LiQ | — |
| | 70 nm | 5 nm | 90 nm | (90%:10%) | 10 nm | (50%:50%) | |
| | | | | 30 nm | | 30 nm | |
| C2 | SpA1 | HATCN | SpMA1 | VG-1:TEG1 | ST2 | ST2:LiQ | — |
| | 70 nm | 5 nm | 90 nm | (90%:10%) | 10 nm | (50%:50%) | |
| | | | | 30 nm | | 30 nm | |
| C3 | SpA1 | HATCN | SpMA1 | VG-3:TEG1 | ST2 | ST2:LiQ | — |
| | 70 nm | 5 nm | 90 nm | (90%:10%) | 10 nm | (50%:50%) | |
| | | | | 30 nm | | 30 nm | |
| C4 | SpA1 | HATCN | SpMA1 | IC1:TEG1 | — | VG-3 | LiQ |
| | 70 nm | 5 nm | 90 nm | (90%:10%) | | 40 nm | 3 nm |
| | | | | 30 nm | | | |
| I1 | SpA1 | HATCN | SpMA1 | INV-2:TEG1 | ST2 | ST2:LiQ | — |
| | 70 nm | 5 nm | 90 nm | (90%:10%) | 10 nm | (50%:50%) | |
| | | | | 30 nm | | 30 nm | |
| I2 | SpA1 | HATCN | SpMA1 | INV-1:TEG1 | ST2 | ST2:LiQ | — |
| | 70 nm | 5 nm | 90 nm | (90%:10%) | 10 nm | (50%:50%) | |
| | | | | 30 nm | | 30 nm | |
| I3 | SpA1 | HATCN | SpMA1 | INV-4:TEG1 | ST2 | ST2:LiQ | — |
| | 70 nm | 5 nm | 90 nm | (90%:10%) | 10 nm | (50%:50%) | |
| | | | | 30 nm | | 30 nm | |
| I4 | SpA1 | HATCN | SpMA1 | INV-3:TEG1 | ST2 | ST2:LiQ | — |
| | 70 nm | 5 nm | 90 nm | (90%:10%) | 10 nm | (50%:50%) | |
| | | | | 30 nm | | 30 nm | |
| I5 | SpA1 | HATCN | SpMA1 | IC1:TEG1 | — | INV-5 | LiQ |
| | 70 nm | 5 nm | 90 nm | (90%:10%) | | 40 nm | 3 nm |
| | | | | 30 nm | | | |
| I6 | SpA1 | HATCN | SpMA1 | IC1:TEG1 | — | INV-2 | LiQ |
| | 70 nm | 5 nm | 90 nm | (90%:10%) | | 30 nm | 3 nm |
| | | | | 30 nm | | | |
| I7 | SpA1 | HATCN | SpMA1 | IC1:TEG1 | — | INV-4 | LiQ |
| | 70 nm | 5 nm | 90 nm | (90%:10%) | | 30 nm | 3 nm |
| | | | | 30 nm | | | |
| I8 | SpA1 | HATCN | SpMA1 | IC1:TEG1 | — | INV-3 | LiQ |
| | 70 nm | 5 nm | 90 nm | (90%:10%) | | 30 nm | 3 nm |
| | | | | 30 nm | | | |
| I9 | SpA1 | HATCN | SpMA1 | INV-2:IC3:TEG1 | IC1 | ST2:LiQ | — |
| | 70 nm | 5 nm | 90 nm | (45%:45%:10%) | 10 nm | (50%:50%) | |
| | | | | 30 nm | | 30 nm | |
| I10 | SpA1 | HATCN | SpMA1 | INV-1:IC2:TEG1 | IC1 | ST2:LiQ | — |
| | 70 nm | 5 nm | 90 nm | (45%:45%:10%) | 10 nm | (50%:50%) | |
| | | | | 30 nm | | 30 nm | |
| I11 | SpA1 | HATCN | SpMA1 | INV-4:IC3:TEG1 | IC1 | ST2:LiQ | — |
| | 70 nm | 5 nm | 90 nm | (45%:45%:10%) | 10 nm | (50%:50%) | |
| | | | | 30 nm | | 30 nm | |
| I12 | SpA1 | HATCN | SpMA1 | INV-3:IC2:TEG1 | IC1 | ST2:LiQ | — |
| | 70 nm | 5 nm | 90 nm | (45%:45%:10%) | 10 nm | (50%:50%) | |
| | | | | 30 nm | | 30 nm | |
| I13 | SpA1 | HATCN | SpMA1 | IC1:TEG1 | INV-2 | ST1:LiQ | — |
| | 70 nm | 5 nm | 90 nm | (90%:10%) | 10 nm | (50%:50%) | |
| | | | | 30 nm | | 30 nm | |
| I14 | SpA1 | HATCN | SpMA1 | IC2:TEG1 | INV-1 | ST2:LiQ | — |
| | 70 nm | 5 nm | 90 nm | (90%:10%) | 10 nm | (50%:50%) | |
| | | | | 30 nm | | 30 nm | |
| I15 | SpA1 | HATCN | SpMA1 | INV-6:TEG1 | ST2 | ST2:LiQ | — |
| | 70 nm | 5 nm | 90 nm | (90%:10%) | 10 nm | (50%:50%) | |
| | | | | 30 nm | | 30 nm | |
| I16 | SpA1 | HATCN | SpMA1 | INV-7:TEG1 | ST2 | ST2:LiQ | — |
| | 70 nm | 5 nm | 90 nm | (90%:10%) | 10 nm | (50%:50%) | |
| | | | | 30 nm | | 30 nm | |
| I17 | SpA1 | HATCN | SpMA1 | INV-8:TEG1 | ST2 | ST2:LiQ | — |
| | 70 nm | 5 nm | 90 nm | (90%:10%) | 10 nm | (50%:50%) | |
| | | | | 30 nm | | 30 nm | |
| I18 | SpA1 | HATCN | SpMA1 | IC1:TEG1 | — | INV-7 | LiQ |
| | 70 nm | 5 nm | 90 nm | (90%:10%) | | 40 nm | 3 nm |
| | | | | 30 nm | | | |
| I19 | SpA1 | HATCN | SpMA1 | IC1:TEG1 | — | INV-8 | LiQ |
| | 70 nm | 5 nm | 90 nm | (90%:10%) | | 40 nm | 3 nm |
| | | | | 30 nm | | | |

TABLE 2
Data of the OLEDs

| | U1000 | EQE | | L₁ | LT |
|---|---|---|---|---|---|
| Ex. | (V) | 1000 | L₀; j₀ | % | (h) |
| C1 | 3.6 | 15.1% | 20 mA/cm² | 80 | 100 |
| C2 | 3.7 | 14.7% | 20 mA/cm² | 80 | 70 |
| C3 | 3.7 | 14.2% | 20 mA/cm² | 80 | 60 |
| C4 | 3.7 | 15.5% | 20 mA/cm² | 80 | 110 |
| I1 | 3.3 | 15.5% | 20 mA/cm² | 80 | 170 |
| I2 | 3.4 | 15.7% | 20 mA/cm² | 80 | 155 |
| I3 | 3.4 | 15.3% | 20 mA/cm² | 80 | 160 |
| I4 | 3.4 | 14.5% | 20 mA/cm² | 80 | 110 |
| I5 | 3.3 | 15.3% | 20 mA/cm² | 80 | 135 |
| I6 | 3.5 | 15.1% | 20 mA/cm² | 80 | 140 |
| I7 | 3.5 | 15.2% | 20 mA/cm² | 80 | 135 |
| I8 | 3.5 | 15.0% | 20 mA/cm² | 80 | 130 |
| I9 | 3.2 | 16.5% | 20 mA/cm² | 80 | 250 |
| I10 | 3.3 | 16.7% | 20 mA/cm² | 80 | 230 |
| I11 | 3.3 | 16.3% | 20 mA/cm² | 80 | 240 |
| I12 | 3.3 | 16.5% | 20 mA/cm² | 80 | 190 |
| I13 | 3.4 | 15.5% | 20 mA/cm² | 80 | 145 |
| I14 | 3.5 | 15.6% | 20 mA/cm² | 80 | 140 |
| I15 | 3.4 | 14.5% | 20 mA/cm² | 80 | 140 |
| I16 | 3.5 | 15.5% | 20 mA/cm² | 80 | 130 |
| I17 | 3.5 | 15.7% | 20 mA/cm² | 80 | 120 |
| I18 | 3.5 | 15.5% | 20 mA/cm² | 80 | 120 |
| I19 | 3.6 | 15.5% | 20 mA/cm² | 80 | 115 |

TABLE 3
Materials used

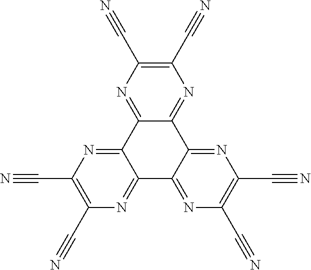 [SMILES: N#Cc1nc2c3nc(C#N)c(C#N)nc3c3nc(C#N)c(C#N)nc3c2nc1C#N]

HATCN

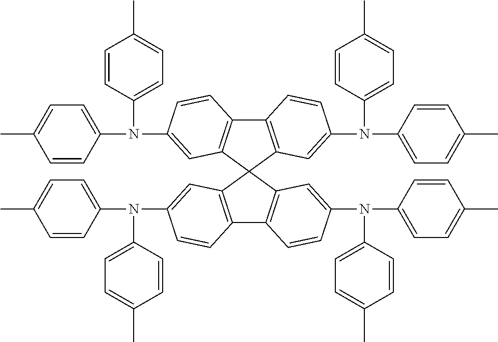

SpA1

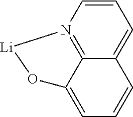

LiQ

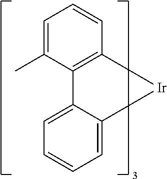

TEG1

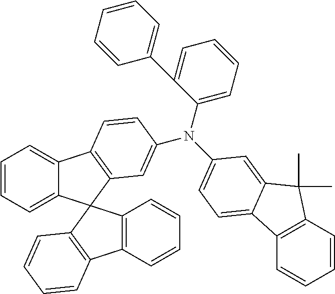 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

SpMA1

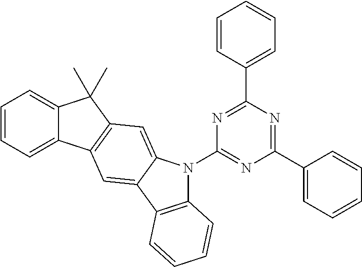 [SMILES: CC1(C)c2ccccc2-c2cc3c4ccccc4n(-c4nc(-c5ccccc5)nc(-c5ccccc5)n4)c3cc21]

IC1

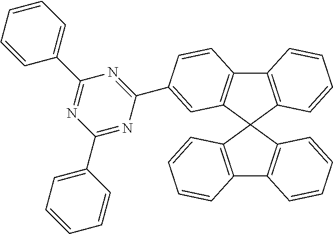 [SMILES: c1ccc(-c2nc(-c3ccccc3)nc(-c3ccc4c(c3)C3(c5ccccc5-c5ccccc53)c3ccccc3-4)n2)cc1]

ST1

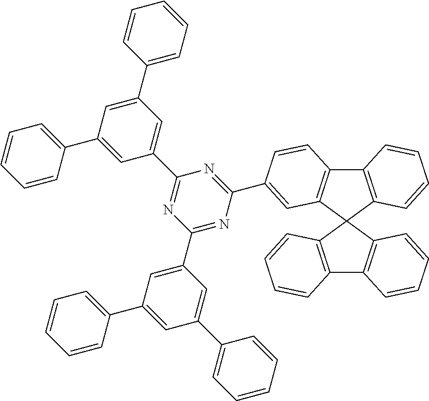 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

ST2

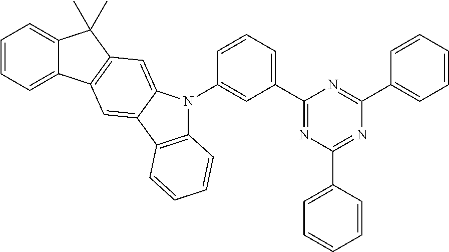 [SMILES: CC1(C)c2ccccc2-c2cc3c4ccccc4n(-c4cccc(-c5nc(-c6ccccc6)nc(-c6ccccc6)n5)c4)c3cc21]

IC2

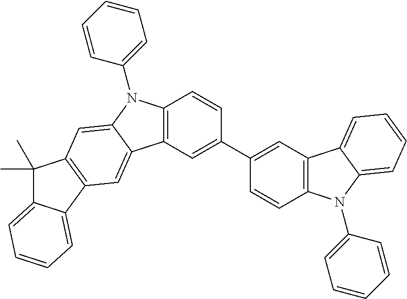 [SMILES: CC1(C)c2ccccc2-c2cc3c4cc(-c5ccc6c(c5)c5ccccc5n6-c5ccccc5)ccc4n(-c4ccccc4)c3cc21]

IC3

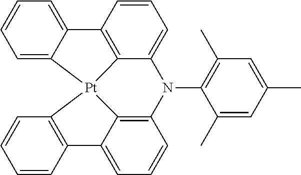

TER1

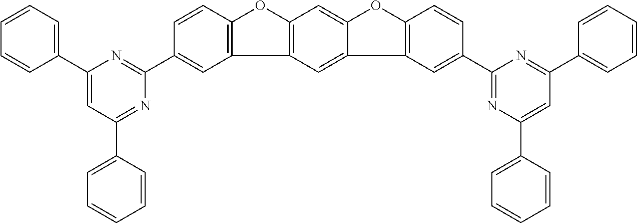 [SMILES: c1ccc(-c2cc(-c3ccccc3)nc(-c3ccc4oc5cc6oc7ccc(-c8nc(-c9ccccc9)cc(-c9ccccc9)n8)cc7c6cc5c4c3)n2)cc1]

VG-1

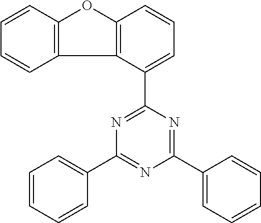 [SMILES: c1ccc(-c2nc(-c3ccccc3)nc(-c3cccc4oc5ccccc5c34)n2)cc1]

VG-2

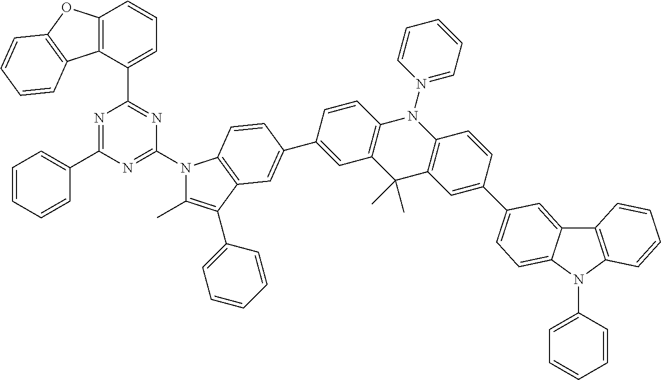

VG-3

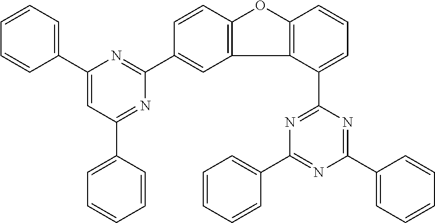 [SMILES: c1ccc(-c2cc(-c3ccccc3)nc(-c3ccc4oc5cccc(-c6nc(-c7ccccc7)nc(-c7ccccc7)n6)c5c4c3)n2)cc1]

INV-1

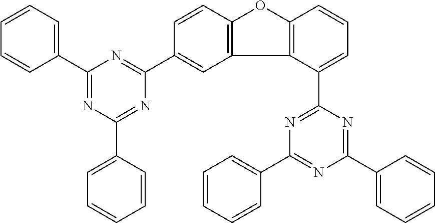 [SMILES: c1ccc(-c2nc(-c3ccccc3)nc(-c3ccc4oc5cccc(-c6nc(-c7ccccc7)nc(-c7ccccc7)n6)c5c4c3)n2)cc1]

INV-2

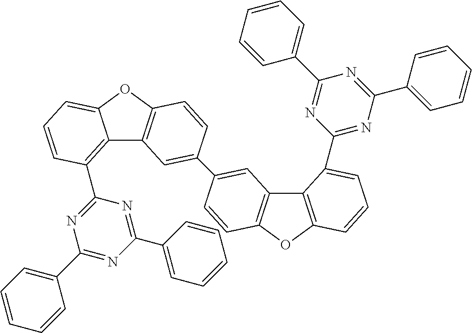 [SMILES: c1ccc(-c2nc(-c3ccccc3)nc(-c3cccc4oc5ccc(-c6ccc7oc8cccc(-c9nc(-c%10ccccc%10)nc(-c%10ccccc%10)n9)c8c7c6)cc5c34)n2)cc1]

INV-3

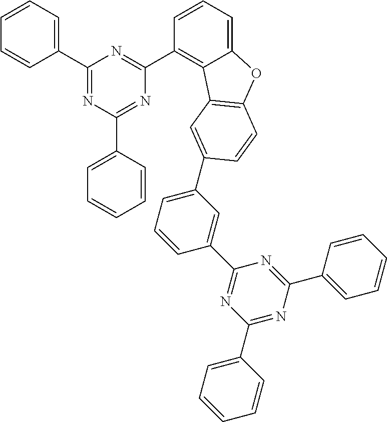 [SMILES: c1ccc(-c2nc(-c3ccccc3)nc(-c3cccc(-c4ccc5oc6cccc(-c7nc(-c8ccccc8)nc(-c8ccccc8)n7)c6c5c4)c3)n2)cc1]

INV-4

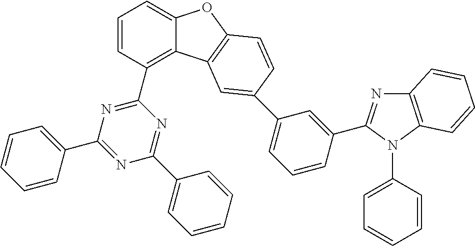 [SMILES: c1ccc(-c2nc(-c3ccccc3)nc(-c3cccc4oc5ccc(-c6cccc(-c7nc8ccccc8n7-c7ccccc7)c6)cc5c34)n2)cc1]

INV-5

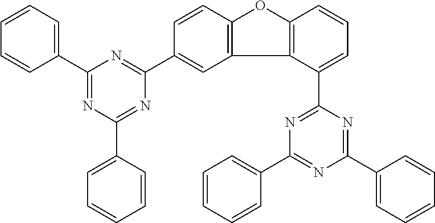 [SMILES: c1ccc(-c2nc(-c3ccccc3)nc(-c3ccc4oc5cccc(-c6nc(-c7ccccc7)nc(-c7ccccc7)n6)c5c4c3)n2)cc1]

INV-6

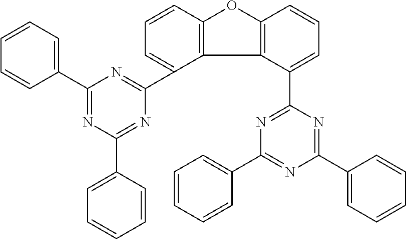 [SMILES: c1ccc(-c2nc(-c3ccccc3)nc(-c3cccc4oc5cccc(-c6nc(-c7ccccc7)nc(-c7ccccc7)n6)c5c34)n2)cc1]

INV-7

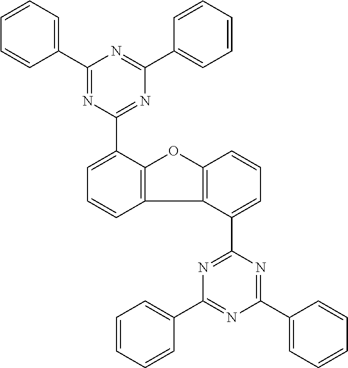 [SMILES: c1ccc(-c2nc(-c3ccccc3)nc(-c3cccc4c3oc3cccc(-c5nc(-c6ccccc6)nc(-c6ccccc6)n5)c34)n2)cc1]

INV-8

1-18. (canceled)

19. A compound of formula (I): 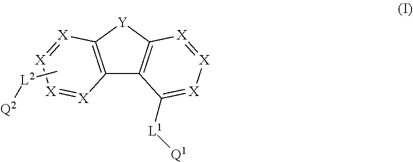
wherein:
Y is O or S;
X is the same or different in each instance and is N or CR¹, with the proviso that not more than two of the X groups in one cycle are N, and C is the attachment site of the L² group;
Q¹ and Q²
are in each case independently an electron-transporting group;
L¹ and L²
are in each case independently a bond or an aromatic or heteroaromatic ring system which has 5 to 30 aromatic ring atoms, which are optionally substituted by one or more R¹ radicals;
R¹ is the same or different in each instance and is H, D, F, Cl, Br, I, B(OR²)₂, CHO, C(═O)R², CR²═C(R²)₂, CN, C(═O)OR², C(═O)N(R²)₂, Si(R²)₃, N(R²)₂, NO₂, P(═O)(R²)₂, OSO₂R², OR², S(═O)R², S(═O)₂R², a straight-chain alkyl, alkoxy, or thioalkoxy group having 1 to 40 carbon atoms or a branched or cyclic alkyl, alkoxy, or thioalkoxy group having 3 to 40 carbon atoms, each of which is optionally substituted by one or more R² radicals, wherein one or more nonadjacent CH₂ groups are optionally replaced by —R²C═CR²—, —C≡C—, Si(R²)₂, C═O, C═S, C═NR², —C(═O)O—, —C(═O)NR²—, NR², P(═O)(R²), —O—, —S—, SO, or SO₂ and wherein one or more hydrogen atoms are optionally replaced by D, F, Cl, Br, I, CN, or NO₂, or an aromatic or heteroaromatic ring system which has 5 to 40 aromatic ring atoms and is optionally substituted in each case by one or more R² radicals, or an aryloxy or heteroaryloxy group which has 5 to 40 aromatic ring atoms and is optionally substituted by one or more R² radicals, or a combination of these systems; and wherein two or more adjacent R¹ substituents together optionally define a mono- or polycyclic, aliphatic or aromatic ring system;
R² is the same or different in each instance and is H, D, F, Cl, Br, I, B(OR³)₂, CHO, C(═O)R³, CR³═C(R³)₂, CN, C(═O)OR³, C(═O)N(R³)₂, Si(R³)₃, N(R³)₂, NO₂, P(═O)(R³)₂, OSO₂R³, OR³, S(═O)R³, S(═O)₂R³, a straight-chain alkyl, alkoxy, or thioalkoxy group having 1 to 40 carbon atoms or a branched or cyclic alkyl, alkoxy, or thioalkoxy group having 3 to 40 carbon atoms, each of which is optionally substituted by one or more R³ radicals, wherein one or more nonadjacent CH₂ groups are optionally replaced by —R³C═CR³—, —C≡C—, Si(R³)₂, C═O, C═S, C═NR³, —C(═O)O—, —C(═O)NR³—, NR³, P(═O)(R³), —O—, —S—, SO, or SO₂ and wherein one or more hydrogen atoms are optionally replaced by D, F, Cl, Br, I, CN, or NO₂, or an aromatic or heteroaromatic ring system which has 5 to 40 aromatic ring atoms and is optionally substituted in each case by one or more R³ radicals, or an aryloxy or heteroaryloxy group which has 5 to 40 aromatic ring atoms and is optionally substituted by one or more R³ radicals, or a combination of these systems; and wherein two or more adjacent R² substituents together optionally define a mono- or polycyclic, aliphatic or aromatic ring system;
R³ is the same or different in each instance and is H, D, F, or an aliphatic, aromatic and/or heteroaromatic hydrocarbyl radical having 1 to 20 carbon atoms, wherein hydrogen atoms are optionally replaced by F; and wherein two or more adjacent R³ substituents together optionally define a mono- or polycyclic, aliphatic or aromatic ring system.

20. The compound of claim 19, wherein the compound is a compound of formula (II):

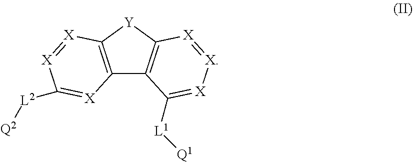

21. The compound of claim 19, wherein the compound is a compound of formula (Ia) 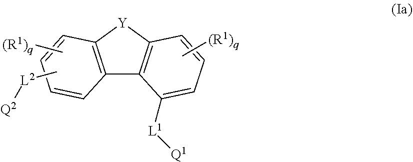
wherein q is 0, 1, or 2.

22. The compound of claim 19, wherein Q¹ and/or Q² comprise at least one structure selected from the group consisting of of pyridines, pyrimidines, pyrazines, pyridazines, triazines, quinazolines, quinoxalines, quinolines, isoquinolines, imidazoles, and benzimidazoles.

23. The compound of claim 19, wherein Q is a heteroaromatic ring system which has 6 to 10 ring atoms and is optionally substituted by one or more R¹ radicals.

24. The compound of claim 19, wherein Q¹ and/or Q² are selected from the group consisting of structures of formulae (Q-1), (Q-2), and (Q-3): 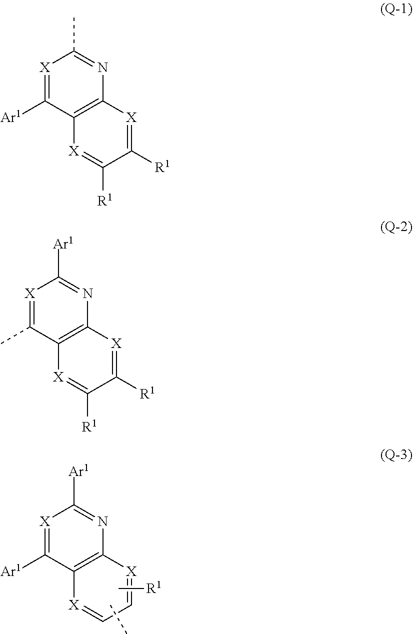
wherein
the dotted bond marks the attachment position; and
Ar¹ is an aromatic or heteroaromatic ring system which has 6 to 40 carbon atoms and is optionally substituted in each case by one or more R² radicals, an aryloxy group which has 5 to 60 aromatic ring atoms and is optionally substituted by one or more R² radicals, or an aralkyl group which has 5 to 60 aromatic ring atoms and is optionally substituted in each case by one or more R² radicals, and wherein two or more adjacent R¹ and/or R² substituents optionally define a mono- or polycyclic aliphatic ring system which is optionally substituted by one or more R³ radicals.

25. The compound of claim 24, wherein Q¹ and/or Q² are selected from the group consisting of structures of formulae (Q-4), (Q-5), (Q-6), (Q-7), (Q-8), (Q-9), (Q-10), (Q-11), (Q-12), and (Q-13): 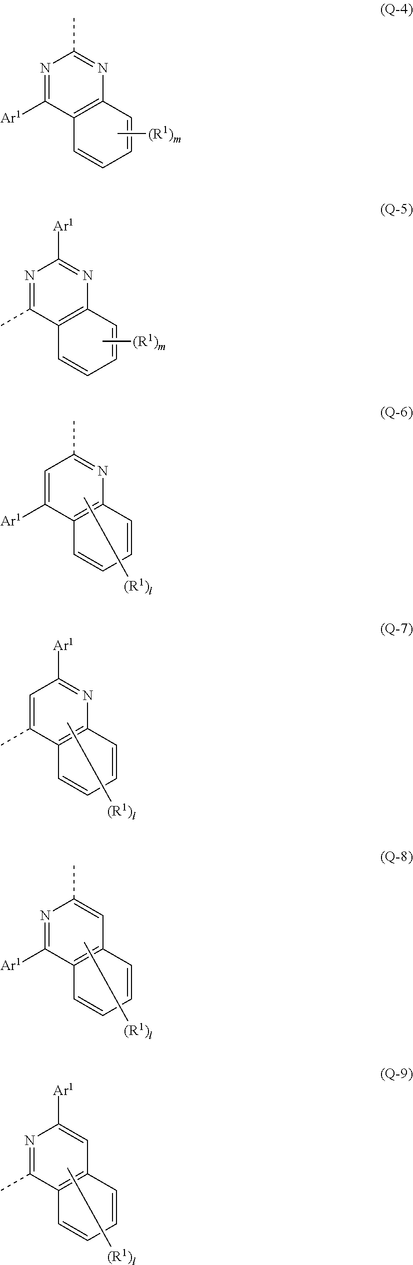 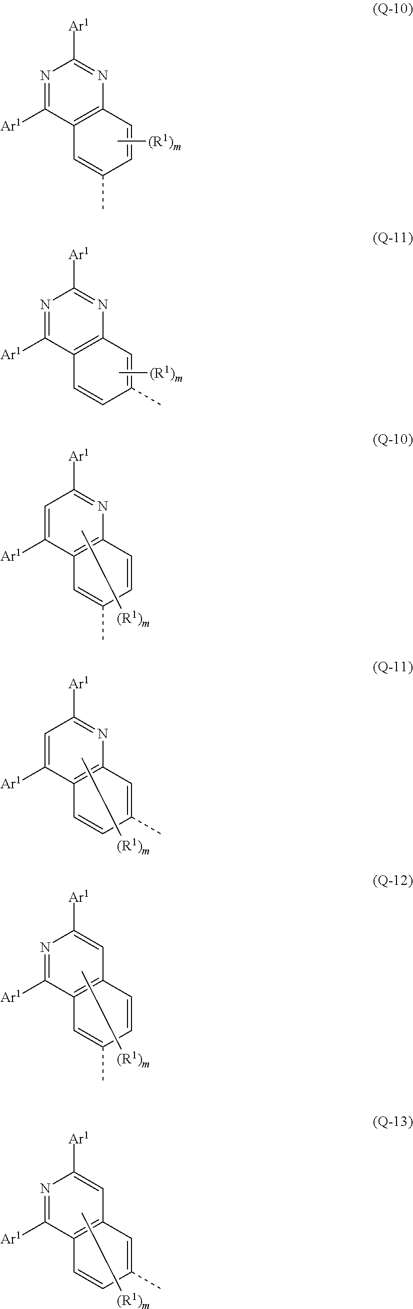
wherein
the dotted bond marks the attachment position;
l is 1, 2, 3, 4 or 5;
m is 0, 1, 2, 3 or 4; and
n is 0, 1, 2 or 3.

26. The compound of claim 24, wherein Q¹ and/or Q² are selected from the group consisting of structures of formulae (Q-14), (Q-15), (Q-16), and (Q-17): 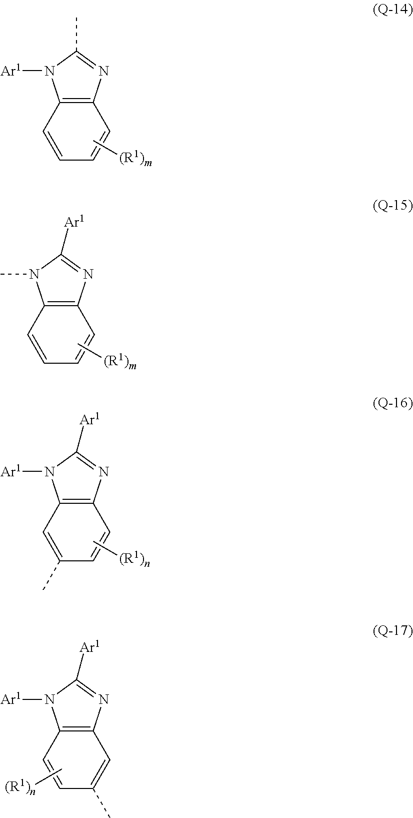
wherein
the dotted bond marks the attachment position;
m is 0, 1, 2, 3 or 4; and
n is 0, 1, 2 or 3.

27. The compound of claim 19, wherein Q¹ and/or Q² group are selected from the group consisting of structures of formulae (Q-18), (Q-19), (Q-20), (Q-21), (Q-22) (Q-23), (Q-24), (Q-25), (Q-26), (Q-27), and (Q-28): 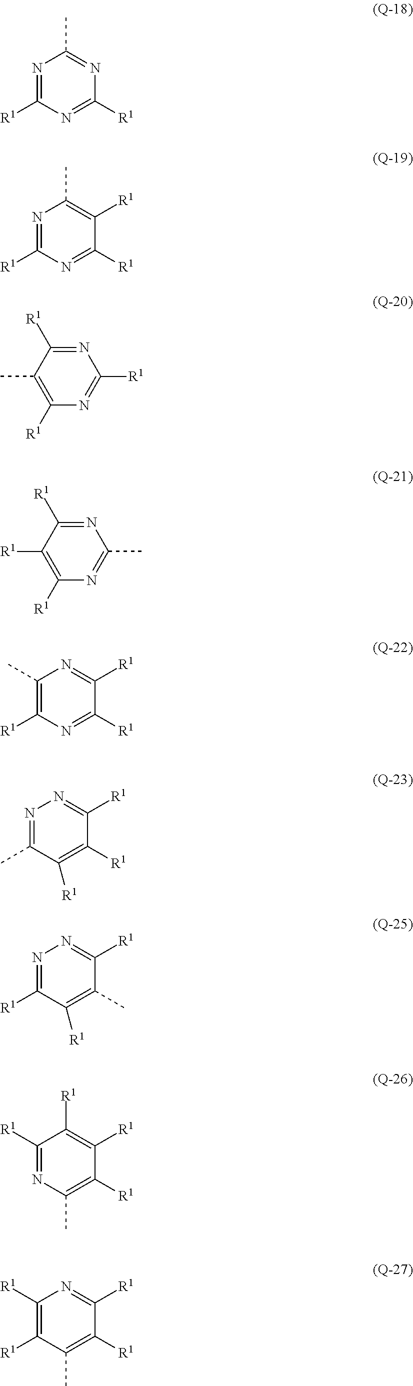 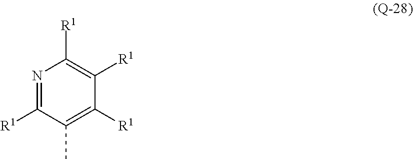
wherein the dotted bond marks the attachment position.

28. The compound of claim 24, wherein Ar¹ is an aromatic ring system which has 6 to 12 aromatic ring atoms and is optionally substituted by one or more R² radicals.

29. The compound of claim 19, wherein the compound does not comprise any carbazole and/or triarylamine groups.

30. The compound of claim 24, wherein the compound does not comprise any hole-transporting groups.

31. An oligomer, polymer, or dendrimer comprising one or more compounds of claim 19, wherein one or more bonds of the compound to the polymer, oligomer, r or dendrimer are present.

32. A composition comprising at least one compound of claim 19 and at least one further compound selected from the group consisting of fluorescent emitters, phosphorescent emitters, host materials, matrix materials, electron transport materials, electron injection materials, hole conductor materials, hole injection materials, n-dopants, wide band gap materials, electron blocker materials, and hole blocker materials.

33. A composition comprising at least one oligomer, polymer, or dendrimer of claim 31 and at least one further compound selected from the group consisting of fluorescent emitters, phosphorescent emitters, host materials, matrix materials, electron transport materials, electron injection materials, hole conductor materials, hole injection materials, n-dopants, wide band gap materials, electron blocker material, and hole blocker materials.

34. A formulation comprising at least one compound of claim 19 and at least one solvent.

35. A formulation comprising at least one oligomer, polymer, or dendrimer of claim 31 and at least one solvent.

36. A process for preparing a compound of claim 19, comprising joining a compound comprising at least one electron-transporting group to a compound comprising at least one benzofuran and/or benzothiophene radical in a coupling reaction.

37. A process for preparing an oligomer, polymer or dendrimer of claim 31, comprising joining a compound comprising at least one electron-transporting group to a compound comprising at least one benzofuran and/or benzothiophene radical in a coupling reaction.

38. An electronic device comprising at least one compound of claim 19.

39. The electronic device of claim 38, wherein the electronic device is selected from the group consisting of organic electroluminescent devices, organic integrated circuits, organic field-effect transistors, organic thin-film transistors, organic light-emitting transistors, organic solar cells, organic optical detectors, organic photoreceptors, organic field quench devices, light-emitting electrochemical cells, and organic laser diodes.

40. An electronic device comprising at least one oligomer, polymer or dendrimer of claim 31.

41. The electronic device of claim 40, wherein the electronic device is selected from the group consisting of organic electroluminescent devices, organic integrated circuits, organic field-effect transistors, organic thin-film transistors, organic light-emitting transistors, organic solar cells, organic optical detectors, organic photoreceptors, organic field quench devices, light-emitting electrochemical cells and organic laser diodes.
